SEQUENCE LISTING (203 sequences)

SEQ ID NO: 1 - DNA, 16 bp, Artificial Sequence
  Primer
ccgtgcagctcatcat

SEQ ID NO: 2 - DNA, 17 bp, Artificial Sequence
  Primer
accgtgcagctcatcat

SEQ ID NO: 3 - DNA, 19 bp, Artificial Sequence
  Primer
tattgtctttgtgttcccg

SEQ ID NO: 4 - DNA, 20 bp, Artificial Sequence
  Primer
atattgtctttgtgttcccg

SEQ ID NO: 5 - DNA, 19 bp, Artificial Sequence
  Primer
cagctcatcacgcagctca

SEQ ID NO: 6 - DNA, 19 bp, Artificial Sequence
  Primer
cctgtgctaggtcttttgc

SEQ ID NO: 7 - DNA, 19 bp, Artificial Sequence
  Primer
catgaatgcgatcttgagt

SEQ ID NO: 8 - DNA, 16 bp, Artificial Sequence
  Primer
tcacagattttgggcg

SEQ ID NO: 9 - DNA, 17 bp, Artificial Sequence
  Primer
atcacagattttgggcg

SEQ ID NO: 10 - DNA, 18 bp, Artificial Sequence
  Primer
gatcacagattttgggcg

SEQ ID NO: 11 - DNA, 19 bp, Artificial Sequence
  Primer
agatcacagattttgggcg

SEQ ID NO: 12 - DNA, 20 bp, Artificial Sequence
  Primer
ctccttctgcatggtattct

SEQ ID NO: 13 - DNA, 19 bp, Artificial Sequence
  Primer
tcacagattttgggctggc

SEQ ID NO: 14 - DNA, 20 bp, Artificial Sequence
  Primer
atcacagattttgggctggc

SEQ ID NO: 15 - DNA, 17 bp, Artificial Sequence
  Primer
ggtctcctggtagtgtg

SEQ ID NO: 16 - DNA, 18 bp, Artificial Sequence
  Primer
tcctcattcactgtccca

SEQ ID NO: 17 - DNA, 17 bp, Artificial Sequence
  Primer
ctcttccgcacccagct

SEQ ID NO: 18 - DNA, 18 bp, Artificial Sequence
  Primer
tctcttccgcacccagct

SEQ ID NO: 19 - DNA, 20 bp, Artificial Sequence
  Primer
gtgaaaacaccgcagcatgt

SEQ ID NO: 20 - DNA, 19 bp, Artificial Sequence
  Primer
tccgcacccagcagtttgg

SEQ ID NO: 21 - DNA, 20 bp, Artificial Sequence
  Primer
tccgcacccagcagtttggc

SEQ ID NO: 22 - DNA, 19 bp, Artificial Sequence
  Primer
gatgctctccagacattct

SEQ ID NO: 23 - DNA, 19 bp, Artificial Sequence
  Primer
ctgttcccaaagcagctct

SEQ ID NO: 24 - DNA, 19 bp, Artificial Sequence
  Primer
aaaaagatcaaagtgctga

SEQ ID NO: 25 - DNA, 20 bp, Artificial Sequence
  Primer
caaaaagatcaaagtgctga

SEQ ID NO: 26 - DNA, 19 bp, Artificial Sequence
  Primer
ccagggaccttaccttata

SEQ ID NO: 27 - DNA, 18 bp, Artificial Sequence
  Primer
atcaaagtgctgggctcc

SEQ ID NO: 28 - DNA, 19 bp, Artificial Sequence
  Primer
gatcaaagtgctgggctcc

SEQ ID NO: 29 - DNA, 19 bp, Artificial Sequence
  Primer
gtcctgtgagaccaattca

SEQ ID NO: 30 - DNA, 18 bp, Artificial Sequence
  Primer
catggcaaggtcaatggc

SEQ ID NO: 31 - DNA, 18 bp, Artificial Sequence
  Primer
aaagatcaaagtgctggc

SEQ ID NO: 32 - DNA, 19 bp, Artificial Sequence
  Primer
aaaagatcaaagtgctggc

SEQ ID NO: 33 - DNA, 20 bp, Artificial Sequence
  Primer
aaaaagatcaaagtgctggc

SEQ ID NO: 34 - DNA, 16 bp, Artificial Sequence
  Primer
tgggggttgtccacga

SEQ ID NO: 35 - DNA, 17 bp, Artificial Sequence
  Primer
gtgggggttgtccacga

SEQ ID NO: 36 - DNA, 18 bp, Artificial Sequence
  Primer
agccacactgacgtgcct

SEQ ID NO: 37 - DNA, 19 bp, Artificial Sequence
  Primer
tgggggttgtccacgctgg

SEQ ID NO: 38 - DNA, 19 bp, Artificial Sequence
  Primer
gatcgcaaaggtaatcagg

SEQ ID NO: 39 - DNA, 18 bp, Artificial Sequence
  Primer
tcttgctatcccaggagc

SEQ ID NO: 40 - DNA, 19 bp, Artificial Sequence
  Primer
aaggaattaagagaagcaa

SEQ ID NO: 41 - DNA, 19 bp, Artificial Sequence
  Primer
actcacatcgaggatttcc

SEQ ID NO: 42 - DNA, 21 bp, Artificial Sequence
  Primer
ggaattaagagaagcaacatc

SEQ ID NO: 43 - DNA, 18 bp, Artificial Sequence
  Primer
cttagagacagcactggc

SEQ ID NO: 44 - DNA, 18 bp, Artificial Sequence
  Primer
cagtaattgcctgtttcc

SEQ ID NO: 45 - DNA, 16 bp, Artificial Sequence
  Primer
ggttgtccacgctggc

SEQ ID NO: 46 - DNA, 16 bp, Artificial Sequence
  Primer
gggttgtccacgctgg

SEQ ID NO: 47 - DNA, 16 bp, Artificial Sequence
  Primer
ggggttgtccacgctg

SEQ ID NO: 48 - DNA, 16 bp, Artificial Sequence
  Primer
gggggttgtccacgct

SEQ ID NO: 49 - DNA, 16 bp, Artificial Sequence
  Primer
tgggggttgtccacgc

SEQ ID NO: 50 - DNA, 18 bp, Artificial Sequence
  Primer
acactgacgtgcctctcc

SEQ ID NO: 51 - DNA, 17 bp, Artificial Sequence
  Primer
acactgacgtgcctctc

SEQ ID NO: 52 - DNA, 17 bp, Artificial Sequence
  Primer
tgacgtgcctctccctc

SEQ ID NO: 53 - DNA, 18 bp, Artificial Sequence
  Primer
tgggggttgtccacgctg

SEQ ID NO: 54 - DNA, 18 bp, Artificial Sequence
  Primer
ggagataaggagccagga

SEQ ID NO: 55 - DNA, 19 bp, Artificial Sequence
  Primer
gcaaactcttgctatccca

SEQ ID NO: 56 - DNA, 22 bp, Artificial Sequence
  Primer
cctccaccgtgcagctcatcat

SEQ ID NO: 57 - DNA, 21 bp, Artificial Sequence
  Primer
ctccaccgtgcagctcatcat

SEQ ID NO: 58 - DNA, 20 bp, Artificial Sequence
  Primer
tccaccgtgcagctcatcat

SEQ ID NO: 59 - DNA, 19 bp, Artificial Sequence
  Primer
ccaccgtgcagctcatcat

SEQ ID NO: 60 - DNA, 18 bp, Artificial Sequence
  Primer
caccgtgcagctcatcat

SEQ ID NO: 61 - DNA, 22 bp, Artificial Sequence
  Primer
cagccgaagggcatgagctgca

SEQ ID NO: 62 - DNA, 21 bp, Artificial Sequence
  Primer
agccgaagggcatgagctgca

SEQ ID NO: 63 - DNA, 20 bp, Artificial Sequence
  Primer
gccgaagggcatgagctgca

SEQ ID NO: 64 - DNA, 19 bp, Artificial Sequence
  Primer
ccgaagggcatgagctgca

SEQ ID NO: 65 - DNA, 18 bp, Artificial Sequence
  Primer
cgaagggcatgagctgca

SEQ ID NO: 66 - DNA, 17 bp, Artificial Sequence
  Primer
gaagggcatgagctgca

SEQ ID NO: 67 - DNA, 16 bp, Artificial Sequence
  Primer
aagggcatgagctgca

SEQ ID NO: 68 - DNA, 20 bp, Artificial Sequence
  Primer
agcaggtactgggagccaat

SEQ ID NO: 69 - DNA, 19 bp, Artificial Sequence
  Primer
agcaggtactgggagccaa

SEQ ID NO: 70 - DNA, 20 bp, Artificial Sequence
  Primer
gttgagcaggtactgggagc

SEQ ID NO: 71 - DNA, 20 bp, Artificial Sequence
  Primer
ccagttgagcaggtactggg

SEQ ID NO: 72 - DNA, 20 bp, Artificial Sequence
  Primer
ctttgtgttcccggacatag

SEQ ID NO: 73 - DNA, 19 bp, Artificial Sequence
  Primer
caccagttgagcaggtact

SEQ ID NO: 74 - DNA, 20 bp, Artificial Sequence
  Primer
atattgtctttgtgttcccg

SEQ ID NO: 75 - DNA, 19 bp, Artificial Sequence
  Primer
tattgtctttgtgttcccg

SEQ ID NO: 76 - DNA, 20 bp, Artificial Sequence
  Primer
cgtggacaacccccacgtgt

SEQ ID NO: 77 - DNA, 19 bp, Artificial Sequence
  Primer
gtggacaacccccacgtgt

SEQ ID NO: 78 - DNA, 18 bp, Artificial Sequence
  Primer
tggacaacccccacgtgt

SEQ ID NO: 79 - DNA, 17 bp, Artificial Sequence
  Primer
ggacaacccccacgtgt

SEQ ID NO: 80 - DNA, 16 bp, Artificial Sequence
  Primer
gacaacccccacgtgt

SEQ ID NO: 81 - DNA, 18 bp, Artificial Sequence
  Primer
cgtggacaacccccacgt

SEQ ID NO: 82 - DNA, 17 bp, Artificial Sequence
  Primer
cgtggacaacccccacg

SEQ ID NO: 83 - DNA, 16 bp, Artificial Sequence
  Primer
cgtggacaacccccac

SEQ ID NO: 84 - DNA, 23 bp, Artificial Sequence
  Primer
gtgcagctcatcacgcagctcat

SEQ ID NO: 85 - DNA, 21 bp, Artificial Sequence
  Primer
tgcagctcatcacgcagctca

SEQ ID NO: 86 - DNA, 21 bp, Artificial Sequence
  Primer
gcagctcatcacgcagctcat

SEQ ID NO: 87 - DNA, 20 bp, Artificial Sequence
  Primer
cagctcatcacgcagctcat

SEQ ID NO: 88 - DNA, 22 bp, Artificial Sequence
  Primer
tcaagatcacagattttgggcg

SEQ ID NO: 89 - DNA, 21 bp, Artificial Sequence
  Primer
caagatcacagattttgggcg

SEQ ID NO: 90 - DNA, 20 bp, Artificial Sequence
  Primer
aagatcacagattttgggcg

SEQ ID NO: 91 - DNA, 22 bp, Artificial Sequence
  Primer
tccgcacccagcagtttggccc

SEQ ID NO: 92 - DNA, 21 bp, Artificial Sequence
  Primer
ccgcacccagcagtttggccc

SEQ ID NO: 93 - DNA, 20 bp, Artificial Sequence
  Primer
cgcacccagcagtttggccc

SEQ ID NO: 94 - DNA, 19 bp, Artificial Sequence
  Primer
gcacccagcagtttggccc

SEQ ID NO: 95 - DNA, 18 bp, Artificial Sequence
  Primer
cacccagcagtttggccc

SEQ ID NO: 96 - DNA, 17 bp, Artificial Sequence
  Primer
acccagcagtttggccc

SEQ ID NO: 97 - DNA, 16 bp, Artificial Sequence
  Primer
cccagcagtttggccc

SEQ ID NO: 98 - DNA, 20 bp, Artificial Sequence
  Primer
ctgacctaaagccacctcct

SEQ ID NO: 99 - DNA, 23 bp, Artificial Sequence
  Primer
aagatcacagattttgggctggc

SEQ ID NO: 100 - DNA, 22 bp, Artificial Sequence
  Primer
agatcacagattttgggctggc

SEQ ID NO: 101 - DNA, 21 bp, Artificial Sequence
  Primer
gatcacagattttgggctggc

SEQ ID NO: 102 - DNA, 22 bp, Artificial Sequence
  Primer
cagattttgggctggccaaaca

SEQ ID NO: 103 - DNA, 21 bp, Artificial Sequence
  Primer
agattttgggctggccaaaca

SEQ ID NO: 104 - DNA, 20 bp, Artificial Sequence
  Primer
gattttgggctggccaaaca

SEQ ID NO: 105 - DNA, 19 bp, Artificial Sequence
  Primer
attttgggctggccaaaca

SEQ ID NO: 106 - DNA, 18 bp, Artificial Sequence
  Primer
ttttgggctggccaaaca

SEQ ID NO: 107 - DNA, 17 bp, Artificial Sequence
  Primer
tttgggctggccaaaca

SEQ ID NO: 108 - DNA, 16 bp, Artificial Sequence
  Primer
ttgggctggccaaaca

SEQ ID NO: 109 - DNA, 22 bp, Artificial Sequence
  Primer
tctttctcttccgcacccagct

SEQ ID NO: 110 - DNA, 21 bp, Artificial Sequence
  Primer
ctttctcttccgcacccagct

SEQ ID NO: 111 - DNA, 20 bp, Artificial Sequence
  Primer
tttctcttccgcacccagct

SEQ ID NO: 112 - DNA, 19 bp, Artificial Sequence
  Primer
ttctcttccgcacccagct

SEQ ID NO: 113 - DNA, 16 bp, Artificial Sequence
  Primer
tcttccgcacccagct

SEQ ID NO: 114 - DNA, 23 bp, Artificial Sequence
  Primer
tccgcacccagcagtttggccag

SEQ ID NO: 115 - DNA, 22 bp, Artificial Sequence
  Primer
ccgcacccagcagtttggccag

SEQ ID NO: 116 - DNA, 21 bp, Artificial Sequence
  Primer
cgcacccagcagtttggccag

SEQ ID NO: 117 - DNA, 20 bp, Artificial Sequence
  Primer
gcacccagcagtttggccag

SEQ ID NO: 118 - DNA, 19 bp, Artificial Sequence
  Primer
cacccagcagtttggccag

SEQ ID NO: 119 - DNA, 23 bp, Artificial Sequence
  Primer
tccgcacccagcagtttggccag

SEQ ID NO: 120 - DNA, 22 bp, Artificial Sequence
  Primer
tccgcacccagcagtttggcca

SEQ ID NO: 121 - DNA, 21 bp, Artificial Sequence
  Primer
tccgcacccagcagtttggcc

SEQ ID NO: 122 - DNA, 22 bp, Artificial Sequence
  Primer
aggaagcctacgtgatggccat

SEQ ID NO: 123 - DNA, 21 bp, Artificial Sequence
  Primer
ggaagcctacgtgatggccat

SEQ ID NO: 124 - DNA, 20 bp, Artificial Sequence
  Primer
gaagcctacgtgatggccat

SEQ ID NO: 125 - DNA, 19 bp, Artificial Sequence
  Primer
aagcctacgtgatggccat

SEQ ID NO: 126 - DNA, 18 bp, Artificial Sequence
  Primer
agcctacgtgatggccat

SEQ ID NO: 127 - DNA, 17 bp, Artificial Sequence
  Primer
gcctacgtgatggccat

SEQ ID NO: 128 - DNA, 16 bp, Artificial Sequence
  Primer
cctacgtgatggccat

SEQ ID NO: 129 - DNA, 22 bp, Artificial Sequence
  Primer
cacacgtgggggttgtccacga

SEQ ID NO: 130 - DNA, 21 bp, Artificial Sequence
  Primer
acacgtgggggttgtccacga

SEQ ID NO: 131 - DNA, 20 bp, Artificial Sequence
  Primer
cacgtgggggttgtccacga

SEQ ID NO: 132 - DNA, 19 bp, Artificial Sequence
  Primer
acgtgggggttgtccacga

SEQ ID NO: 133 - DNA, 18 bp, Artificial Sequence
  Primer
cgtgggggttgtccacga

SEQ ID NO: 134 - DNA, 19 bp, Artificial Sequence
  Primer
catgtgcccctccttctgg

SEQ ID NO: 135 - DNA, 19 bp, Artificial Sequence
  Primer
agccacactgacgtgcctc

SEQ ID NO: 136 - DNA, 23 bp, Artificial Sequence
  Primer
cacgtgggggttgtccacgctgg

SEQ ID NO: 137 - DNA, 22 bp, Artificial Sequence
  Primer
acgtgggggttgtccacgctgg

SEQ ID NO: 138 - DNA, 21 bp, Artificial Sequence
  Primer
cgtgggggttgtccacgctgg

SEQ ID NO: 139 - DNA, 20 bp, Artificial Sequence
  Primer
gtgggggttgtccacgctgg

SEQ ID NO: 140 - DNA, 22 bp, Artificial Sequence
  Primer
atcaaaaagatcaaagtgctga

SEQ ID NO: 141 - DNA, 21 bp, Artificial Sequence
  Primer
tcaaaaagatcaaagtgctga

SEQ ID NO: 142 - DNA, 18 bp, Artificial Sequence
  Primer
aaaagatcaaagtgctga

SEQ ID NO: 143 - DNA, 17 bp, Artificial Sequence
  Primer
aaagatcaaagtgctga

SEQ ID NO: 144 - DNA, 16 bp, Artificial Sequence
  Primer
aagatcaaagtgctga

SEQ ID NO: 145 - DNA, 22 bp, Artificial Sequence
  Primer
ccgtgccgaacgcaccggagct

SEQ ID NO: 146 - DNA, 21 bp, Artificial Sequence
  Primer
cgtgccgaacgcaccggagct

SEQ ID NO: 147 - DNA, 20 bp, Artificial Sequence
  Primer
gtgccgaacgcaccggagct

SEQ ID NO: 148 - DNA, 19 bp, Artificial Sequence
  Primer
tgccgaacgcaccggagct

SEQ ID NO: 149 - DNA, 18 bp, Artificial Sequence
  Primer
gccgaacgcaccggagct

SEQ ID NO: 150 - DNA, 17 bp, Artificial Sequence
  Primer
ccgaacgcaccggagct

SEQ ID NO: 151 - DNA, 16 bp, Artificial Sequence
  Primer
cgaacgcaccggagct

SEQ ID NO: 152 - DNA, 19 bp, Artificial Sequence
  Primer
ctgtgccagggaccttacc

SEQ ID NO: 153 - DNA, 23 bp, Artificial Sequence
  Primer
caaagtgctgggctccggtgcgt

SEQ ID NO: 154 - DNA, 22 bp, Artificial Sequence
  Primer
caaagtgctgggctccggtgcg

SEQ ID NO: 155 - DNA, 21 bp, Artificial Sequence
  Primer
caaagtgctgggctccggtgc

SEQ ID NO: 156 - DNA, 20 bp, Artificial Sequence
  Primer
caaagtgctgggctccggtg

SEQ ID NO: 157 - DNA, 19 bp, Artificial Sequence
  Primer
caaagtgctgggctccggt

SEQ ID NO: 158 - DNA, 23 bp, Artificial Sequence
  Primer
aaaagatcaaagtgctgggctcc

SEQ ID NO: 159 - DNA, 22 bp, Artificial Sequence
  Primer
aaagatcaaagtgctgggctcc

SEQ ID NO: 160 - DNA, 21 bp, Artificial Sequence
  Primer
aagatcaaagtgctgggctcc

SEQ ID NO: 161 - DNA, 20 bp, Artificial Sequence
  Primer
agatcaaagtgctgggctcc

SEQ ID NO: 162 - DNA, 22 bp, Artificial Sequence
  Primer
tcaaaaagatcaaagtgctggc

SEQ ID NO: 163 - DNA, 21 bp, Artificial Sequence
  Primer
caaaaagatcaaagtgctggc

SEQ ID NO: 164 - DNA, 17 bp, Artificial Sequence
  Primer
aagatcaaagtgctggc

SEQ ID NO: 165 - DNA, 16 bp, Artificial Sequence
  Primer
agatcaaagtgctggc

SEQ ID NO: 166 - DNA, 22 bp, Artificial Sequence
  Primer
accgtgccgaacgcaccggagg

SEQ ID NO: 167 - DNA, 21 bp, Artificial Sequence
  Primer
ccgtgccgaacgcaccggagg

SEQ ID NO: 168 - DNA, 20 bp, Artificial Sequence
  Primer
cgtgccgaacgcaccggagg

SEQ ID NO: 169 - DNA, 19 bp, Artificial Sequence
  Primer
gtgccgaacgcaccggagg

SEQ ID NO: 170 - DNA, 18 bp, Artificial Sequence
  Primer
tgccgaacgcaccggagg

SEQ ID NO: 171 - DNA, 17 bp, Artificial Sequence
  Primer
gccgaacgcaccggagg

SEQ ID NO: 172 - DNA, 16 bp, Artificial Sequence
  Primer
ccgaacgcaccggagg

SEQ ID NO: 173 - DNA, 20 bp, Artificial Sequence
  Primer
cccgtcgctatcaaggaatt

SEQ ID NO: 174 - DNA, 20 bp, Artificial Sequence
  Primer
atcaaggaattaagagaagc

SEQ ID NO: 175 - DNA, 20 bp, Artificial Sequence
  Primer
ctttcggagatgttgcttct

SEQ ID NO: 176 - DNA, 20 bp, Artificial Sequence
  Primer
atgttgcttctcttaattcc

SEQ ID NO: 177 - DNA, 19 bp, Artificial Sequence
  Primer
ttgcttctcttaattcctt

SEQ ID NO: 178 - DNA, 23 bp, Artificial Sequence
  Primer
tcaaggaattaagagaagcaaca

SEQ ID NO: 179 - DNA, 22 bp, Artificial Sequence
  Primer
caaggaattaagagaagcaaca

SEQ ID NO: 180 - DNA, 21 bp, Artificial Sequence
  Primer
aaggaattaagagaagcaaca

SEQ ID NO: 181 - DNA, 20 bp, Artificial Sequence
  Primer
aggaattaagagaagcaaca

SEQ ID NO: 182 - DNA, 19 bp, Artificial Sequence
  Primer
ggaattaagagaagcaaca

SEQ ID NO: 183 - DNA, 21 bp, Artificial Sequence
  Primer
tcaaggaattaagagaagcaa

SEQ ID NO: 184 - DNA, 20 bp, Artificial Sequence
  Primer
tcaaggaattaagagaagca

SEQ ID NO: 185 - DNA, 19 bp, Artificial Sequence
  Primer
tcaaggaattaagagaagc

SEQ ID NO: 186 - DNA, 18 bp, Artificial Sequence
  Primer
tcaaggaattaagagaag

SEQ ID NO: 187 - DNA, 23 bp, Artificial Sequence
  Primer
ggaattaagagaagcaacatctc

SEQ ID NO: 188 - DNA, 22 bp, Artificial Sequence
  Primer
ggaattaagagaagcaacatct

SEQ ID NO: 189 - DNA, 20 bp, Artificial Sequence
  Primer
ggaattaagagaagcaacat

SEQ ID NO: 190 - DNA, 19 bp, Artificial Sequence
  Primer
ggaattaagagaagcaaca

SEQ ID NO: 191 - DNA, 19 bp, Artificial Sequence
  Primer
cacactgacgtgcctctcc

SEQ ID NO: 192 - DNA, 23 bp, Artificial Sequence
  Primer
cacgtgggggttgtccacgctgg

SEQ ID NO: 193 - DNA, 22 bp, Artificial Sequence
  Primer
acgtgggggttgtccacgctgg

SEQ ID NO: 194 - DNA, 21 bp, Artificial Sequence
  Primer
cgtgggggttgtccacgctgg

SEQ ID NO: 195 - DNA, 20 bp, Artificial Sequence
  Primer
gtgggggttgtccacgctgg

SEQ ID NO: 196 - DNA, 19 bp, Artificial Sequence
  Primer
tgggggttgtccacgctgg

SEQ ID NO: 197 - DNA, 404 bp, Homo sapiens
aaggggtccatgtgcccctccttctggccaccatgcgaagccacactgacgtgcctctccctccctccaggaagcctacgtgatggccagcgtggacaacccccacgtgtgccgcctgctgggcatctgcctcacctccaccgtgcagctcatcatgcagctcatgcccttcggctgcctcctggactatgtccgggaacacaaagacaatattggctcccagtacctgctcaactggtgtgtgcagatcgcaaaggtaatcagggaagggagatacggggaggggagataaggagccaggatcctcacatgcggtctgcgctcctgggatagcaagagtttgccatggggatatgtgtgtgcgtgcatgcagcacacacacattcctttattttggattcaat

SEQ ID NO: 198 - DNA, 301 bp, Homo sapiens
agatcacagattttgggcgggccaaactgctgggtgcggaagagaaagaataccatgcagaaggaggcaaagtaaggaggtggctttaggtcagccagcattttcctgacaccagggaccaggctgccttcccactagctgtattgtttaacacatgcaggggaggatgctctccagacattctgggtgagctcgcagcagctgctgctggcagctgggtccagccagggtctcctggtagtgtgagccagagctgctttgggaacagtacttgctgggacagtgaatgaggatgttatcc

SEQ ID NO: 199 - DNA, 300 bp, Homo sapiens
gtgaaaacaccgcagcatgtcaagatcacagattttgggctggccaaacagctgggtgcggaagagaaagaataccatgcagaaggaggcaaagtaaggaggtggctttaggtcagccagcattttcctgacaccagggaccaggctgccttcccactagctgtattgtttaacacatgcaggggaggatgctctccagacattctgggtgagctcgcagcagctgctgctggcagctgggtccagccagggtctcctggtagtgtgagccagagctgctttgggaacagtacttgctgg

SEQ ID NO: 200 - DNA, 301 bp, Homo sapiens
agccacactgacgtgcctctccctccctccaggaagcctacgtgatggccatcgtggacaacccccacgtgtgccgcctgctgggcatctgcctcacctccaccgtgcagctcatcacgcagctcatgcccttcggctgcctcctggactatgtccgggaacacaaagacaatattggctcccagtacctgctcaactggtgtgtgcagatcgcaaaggtaatcagggaagggagatacggggaggggagataaggagccaggatcctcacatgcggtctgcgctcctgggatagcaagag

SEQ ID NO: 201 - DNA, 306 bp, Homo sapiens
caaaaagatcaaagtgctgagctccggtgcgttcggcacggtgtataaggtaaggtccctggcacaggcctctgggctgggccgcagggcctctcatggtctggtggggagcccagagtccttgcaagctgtatatttccatcatctactttactctttgtttcactgagtgtttgggaaactccagtgtttttcccaagttattgagaggaaatcttttataaccacagtaatcagtggtcctgtgagaccaattcacagaccaaaggcatttttatgaaaggggccattgaccttgccatgggg

SEQ ID NO: 202 - DNA, 301 bp, Homo sapiens
atcaaggaattaagagaagcaacatctccgaaagccaacaaggaaatcctcgatgtgagtttctgctttgctgtgtgggggtccatggctctgaacctcaggcccaccttttctcatgtctggcagctgctctgctctagaccctgctcatctccacatcctaaatgttcactttctatgtctttccctttctagctctagtgggtataactccctccccttagagacagcactggcctctcccatgctggtatccaccccaaaaggctggaaacaggcaattactggcatctacccagca

SEQ ID NO: 203 - DNA, 301 bp, Homo sapiens
tgacgtgcctctccctccctccaggaagcctacgtgatggccagcgtggacaacccccacgtgtgccgcctgctgggcatctgcctcacctccaccgtgcagctcatcacgcagctcatgcccttcggctgcctcctggactatgtccgggaacacaaagacaatattggctcccagtacctgctcaactggtgtgtgcagatcgcaaaggtaatcagggaagggagatacggggaggggagataaggagccaggatcctcacatgcggtctgcgctcctgggatagcaagagtttgccat

(19) United States
(12) Patent Application Publication
(10) Pub. No.: US 2022/0073993 A1
(43) Pub. Date: Mar. 10, 2022

(54) METHODS AND KITS FOR DETECTING EGFR MUTATIONS

(71) Applicants: DIADX, LES MATELLES (FR); INSERM (INSTITUT NATIONAL DE LA SANTÉ ET DE LA RECHERCHE MÉDICALE), PARIS (FR); UNIVERSITE DE MONTPELLIER, MONTPELLIER (FR); INSTITUT REGIONAL DU CANCER DE MONTPELLIER, MONTPELLIER (FR)

(72) Inventors: ALAIN THIERRY, SAINT MATHIEU DE TREVIERS (FR); BARBARA OTTOLINI, MONTPELLIER (FR)

(21) Appl. No.: 17/254,555

(22) PCT Filed: Jun. 20, 2019
(86) PCT No.: PCT/EP2019/066371

§ 371 Date: Dec. 21, 2020

(30) Foreign Application Priority Data
Jun. 21, 2018 (EP) 18305788.4

(51) Int. Cl. C12Q 1/6886

(52) U.S. Cl. CPC C12Q 1/6886; C12Q 2600/16; C12Q 2600/156

(57) ABSTRACT

The present invention relates to methods, materials and kits for detecting mutations in the EGFR gene. The invention also relates to methods for detecting or diagnosing cancer in a subject, as well as for determining the clinical stage, and clinical progression of a cancer.

### CROSS-REFERENCE TO RELATED APPLICATION

This application is the U.S. national stage application of International Patent Application No. PCT/EP2019/066371, filed Jun. 20, 2019.

### REFERENCE TO SEQUENCE LISTING

The Sequence Listing for this application is labeled “Seq-List.txt” which was created on Dec. 21, 2020 and is 39 KB. The entire content of the sequence listing is incorporated herein by reference in its entirety.

The present invention relates to methods, materials and kits for detecting mutations in the EGFR gene. The invention also relates to methods for detecting or diagnosing cancer in a subject, as well as for determining the clinical stage, and clinical progression of a cancer.

### BACKGROUND

The epidermal growth factor receptor (EGFR) is a trans-membrane tyrosine-kinase protein whose deregulated activation can lead to cellular proliferation, inhibition of apoptosis, angiogenesis and to phenotypic changes towards cell adhesion, migration and invasion. Activating EGFR mutations are clustered within exons 18-21 which encode for a portion of the EGFR kinase domain. EGFR mutations are very frequent in lung adenocarcinoma and are estimated to affect approximately 15% of US patients and more than 30% patients of Asian descent. In this respect, small-molecule EGFR tyrosine kinase inhibitors (TKIs) have been designed and developed, which launched the era of targeted, personalized and precise medicine for lung cancer. EGFR tyrosine kinase inhibitors are associated with high response rate and progression-free survival for patients with non-small cell lung cancer with EGFR-mutant genotype. Iressa® (gefitinib) and Tarceva® (erlotinib) constitute the first generation of EGFR tyrosine kinase inhibitors. However, most patients develop acquired drug resistance mostly driven by the T790M mutation occurring within the exon 20 of the EGFR gene. Table 1 below presents a list of EGFR mutations conferring either sensitivity or resistance to the first-generation TKIs.

TABLE 1

| | | global frequency EGFR mutant: 29% (COSMIC 2015) | | | |
|---|---|---|---|---|---|
| | | | | frequency of each | |
| | | | | mutation in EGFR | R/S TKI |
| EGFR | NSCLC | EXON | Mutation | mutant tumors | EGFR |
| | | 18 | G719A | 0.60% | S |
| | | | G719C | 0.30% | S |
| | | | G719S | 0.50% | S |
| | | 19 | exon 19 DEL | 48% | S |
| | | 20 | S768I | <1% | S |
| | | 20 | exon 20 INS | 4-9.2% | R |
| | | 20 | T790M | <5% untreated tumors | R |
| | | | | >50% of acquired | |
| | | | | resistance to TKIs | |
| | | 21 | L861Q | 2% | S |
| | | 21 | L858R | 43% | S |

Although the second-generation EGFR TKIs, such as Giotrif® (afatinib), dacomitinib and neratinib, demonstrated promising activity against T790M in preclinical models, they have failed to overcome resistance in patients due to dose-limiting toxicity.

Recently, the third-generation EGFR TKIs Tagrisso® (osimertinib) have shown to be effective against cell lines and murine models harbouring T790M mutations whilst sparing EGFR wild-type cells, which represented a promising breakthrough approach in overcoming T790M-mediated resistance in NSCLC patients.

On Mar. 30, 2017, following the positive results obtained by the AURA study (ClinicalTrials.gov identifier: NCT01802632) the U.S. Food and Drug Administration granted regular approval to Tagrisso® for the treatment of patients with metastatic EGFR T790M mutation-positive NSCLC, whose disease has progressed on or after EGFR TKI therapy. Positive EGFR T790M mutation status needs to be determined by an FDA-approved test, either performed on tissue samples or from circulating tumour DNA obtained from plasma samples.

Accurate and sensitive detection systems for prompt genotyping of sensitizing mutations to EGFR TKIs treatments and for the identification of pre-existing or acquired resistance to first-generation TKIs drugs are hence pivotal for providing lung cancer patients with the best and most effective personalized treatment, based on their genetic profiles.

### SUMMARY OF THE INVENTION

The invention provides novel methods, kits and materials allowing fast and reliable detection of mutations in the EGFR gene. The invention more specifically provides methods and kits for optimized detection of mutations in EGFR sequence from cell-free nucleic acids (“cfNA”). The invention is suitable to detect any type of mutation, such as nucleotide substitution(s), deletion(s) and/or insertion(s) in the EGFR gene sequence.

In a first aspect, the invention more particularly relates to a method for determining the presence of a mutation in the EGFR gene in a sample from a subject, the method comprising amplifying, in a sample of a biological fluid from the subject containing cell free nucleic acids, at least a first target sequence from the EGFR gene, the first target sequence containing a site of a mutation on said gene, wherein the first amplified target sequence is 50-95 nt in length and amplification of the first target sequence is conducted with a first set of reagents comprising (i) a mutation-specific primer of 15-25 nt in length, (ii) a paired primer of 15-25 nt in length and (iii) an oligoblocker, wherein the oligoblocker hybridizes specifically to the first target sequence in non-mutated form and blocks polymerase elongation.

In a preferred embodiment, the method further comprises amplifying a second non-mutated target sequence on the EGFR gene, said second target sequence being located between 100 and 400 bp from the first target sequence on said gene, the first and second amplified sequences each being 50-95 nt in length and of a similar length.

In a further aspect, the invention relates to a kit for determining the presence of a mutation in the EGFR gene in a sample of a biological fluid containing cell free nucleic acids from a subject, the kit comprising at least a first set of reagents, wherein the first set of reagents amplifies a first target sequence from the EGFR gene, said first target sequence containing a site of a mutation in the EGFR gene, said first set comprising (i) a mutation-specific primer of 15-25 nt in length, (ii) a paired primer of 15-25 nt in length positioned such that the amplified first target sequence is 50-95 nt in length, and (iii) an oligoblocker, wherein the oligoblocker hybridizes specifically to the first target sequence in non-mutated form and blocks polymerase elongation.

Preferably, the kit comprises a second set of reagents (b) which amplifies a second non-mutated target sequence from the EGFR gene, said second target sequence being located between 100 and 400 bp from the first target sequence on said gene, and wherein said amplified second target sequence is 50-95 nt in length and of a length similar to that of the first amplified target sequence.

The invention may be used preferably to detect a mutation selected from G719A, G719C, G719S, S768I, T790M, L858R, L861Q, exon19DEL and exon20INS, or a combination thereof.

A further object of the invention relates to a nucleic acid molecule consisting of a nucleotide sequence selected from any one of SEQ ID NOs: 1 to 55.

The invention also relates to the use of a nucleic acid molecule as defined above for in vitro amplification of a target sequence in the EGFR gene.

The invention also relates to a method for diagnosing a patient, comprising detecting a mutation in the EGFR gene in a sample from the patient using a method or kit or nucleic acid as defined above.

The invention also relates to a method for treating a patient, comprising detecting a mutation in the EGFR gene in a sample from the patient using a method or kit or nucleic acid as defined above, and treating subjects having cancer.

### BRIEF DESCRIPTION OF THE FIGURES

FIG. 1: Scheme of the method of the invention in conventional (Panel A) and inverse (Panel B) configuration.

FIG. 2: Position of the proposed T790M wild-type primer set in relation with the T790M allele-specific primer set.

FIG. 3: DNA region to target in conventional sense to produce suitable T790M wild-type primer sets.

FIG. 4: Melting curves of commercial DNA mutant for EGFR T790M using the oligonucleotide combination T790M 16+T790 P19+T790 OB19.

FIG. 5: Melting curves of genomic DNA WT for EGFR T790M from the Difi cell line compared to melting curves of commercial DNA mutant for EGFR T790M using the oligonucleotide combination T790M 16+T790 P19+T790 OB19.

FIG. 6: Efficiency comparison between the proposed T790M system and a comparative detection system by Zhao et al. on lung cancer patient number 8. This sample was determined as mutant by our EGFR T790M assay with a mutant allele frequency of 11% and mutant cfDNA concentrations varying from 0.18 to 10 pg/μL in the extract. The compared system did not detect any mutation in this sample.

FIG. 7: Comparison of the ARMS-PCR based system by Zhao et al. without proposed system. Despite the low cfDNA concentration, this sample was determined as mutant by our EGFR T790M assay, with mutant allele frequency of 0.32% and mutant cfDNA concentration of 0.3 pg/μL. The compared system did not detect any mutation in this same sample.

FIG. 8: DNA region to target in conventional sense to produce suitable L858R wild-type primer sets.

FIG. 9: Melting curves of commercial DNA mutant for EGFR L858R using the oligonucleotide combination L858R 19+L858 P20+L858 OB19/3.

FIG. 10: Melting curves of genomic DNA WT for EGFR L858R from the Difi cell line compared to melting curves of commercial DNA mutant for EGFR L858R using the oligonucleotide combination L858R 19+L858 P20+L858 OB19/3.

FIG. 11: DNA region to target in inverse sense to produce suitable L861Q wild-type primer sets.

FIG. 12: Melting curves of commercial DNA mutant for EGFR L861Q using the oligonucleotide combination L861Q i18+L861 iP20+L861 iOB20/2.

FIG. 13: Melting curves of genomic DNA WT for EGFR L861Q from the Difi cell line compared to melting curves of commercial DNA mutant for EGFR L861Q using the oligonucleotide combination L861Q i18+L861 iP20+L861 iOB20/2.

FIG. 14: DNA region to target in inverse sense to produce suitable S768 wild-type primer sets.

FIG. 15: Melting curves of commercial DNA mutant for EGFR S768I using the oligonucleotide combination S768I i16+S768 iP18+S768 iOB19.

FIG. 16: Melting curves of genomic DNA WT for EGFR S768I from the Difi cell line compared to melting curves of commercial DNA mutant for EGFR S768I using the oligonucleotide combination S768I i16+S768 iP18+S768 iOB19.

FIG. 17: Melting curves of commercial DNA mutant for EGFR S768I using the oligonucleotide combination S768I i17+S768 iP18+S768 iOB19.

FIG. 18: Melting curves of genomic DNA WT for EGFR S768I from the Difi cell line compared to melting curves of commercial DNA mutant for EGFR S768I using the oligonucleotide combination S768I i17+S768 iP18+S768 iOB19.

FIG. 19: DNA region to target in conventional sense to produce suitable G719wild-type primer sets. The potential WT primer candidates produce a PCR product of 64 bp.

FIG. 20: Melting curves of commercial DNA mutant for EGFR G719S using the oligonucleotide combination G719S 20+G719 P19+G719 OB19.

FIG. 21: Melting curves of genomic DNA WT for EGFR G719S from the Difi cell line compared to melting curves of commercial DNA mutant for EGFR G719S using the oligonucleotide combination G719S 20+G719 P19+G719 OB19.

FIG. 22: Melting curves of commercial DNA mutant for EGFR G719A using the oligonucleotide combination G719A 18+G719 P19+G719 OB18/2.

FIG. 23: Melting curves of commercial DNA positive for EGFR G719S and WT for EGFR G719A compared to melting curves of commercial DNA mutant for EGFR G719A using the oligonucleotide combination G719A 18+G719 P19+G719 OB18/2.

FIG. 24: Strategy for qualitative detection of the deletion. A. Strategy in conventional configuration: A1 is the primer targeting the beginning of the deletion. When DNA is WT for the deletion, the primer set A1-A2 amplifies the WT region. When DNA is mutant for the deletion, A1 is not hybridized with the DNA sequence and there is no amplification. B. Strategy in inverse configuration: A2 is the primer targeting the beginning of the deletion. When DNA is WT for the deletion, the primer set A1-A2 amplifies the WT region. When DNA is mutant for the deletion, A2 is not hybridized with the DNA sequence and there is no amplification.

FIG. 25: Strategy to improve specificity and sensitivity of deletions by IntPlex system in conventional configuration (A) and inverse configuration (B). The oligoblocker corresponds to the WT DNA sequence. As this oligoblocker is designed to have a higher affinity for the WT DNA sequence than the primer targeting the mutation, the oligoblocker will be hybridized first and will avoid non-specific WT DNA sequence amplification if present at the region of the mutation.

FIG. 26. Strategy to detect qualitatively and quantitatively deletion in EGFR exon 19 in conventional configuration (A) and inverse configuration (B).

FIG. 27: DNA region to target in conventional sense to produce suitable DEL19 wild-type primer sets.

FIG. 28: Melting curves of commercial DNA mutant for EGFR Δ(E746-A750) using the oligonucleotide combination DEL19 17N+DEL19 P19+DEL19 OB21/3.

FIG. 29: DNA region to target in inverse sense to produce suitable Ins20 wild-type primer sets.

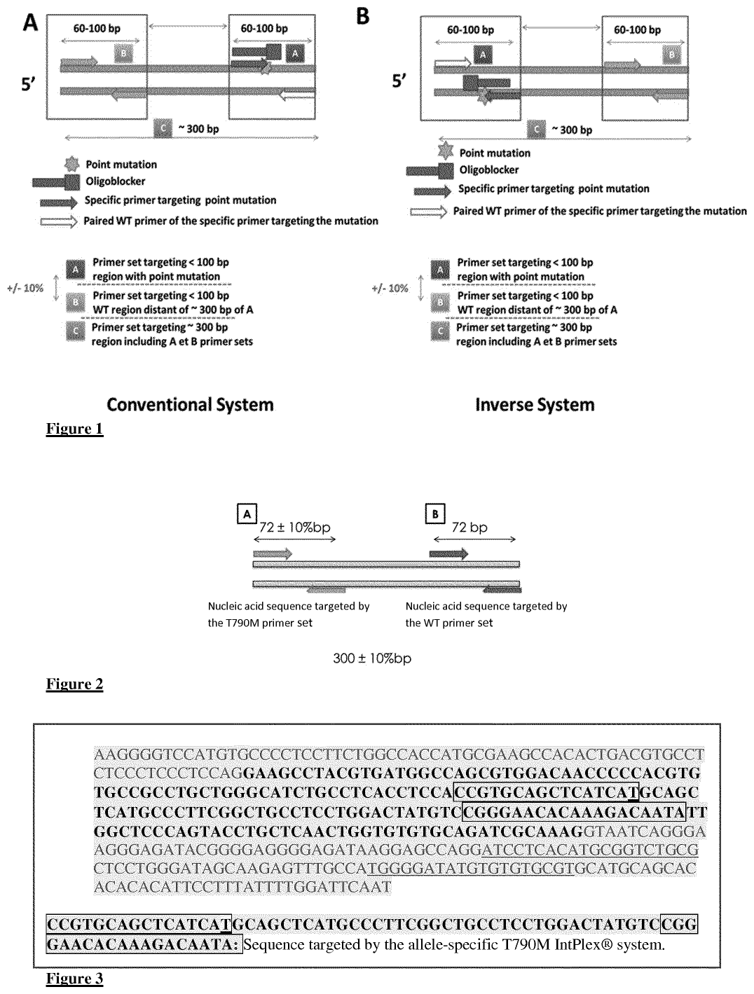

### DETAILED DESCRIPTION

The present invention relates to improved methods and kits for detecting mutations in EGFR gene sequence, as well as the uses thereof. EGFR mutations are correlated to cancers, particularly lung cancer, and the invention may be used e.g., in the diagnosis, drug development, or clinical monitoring of subjects with cancer, as well as to adjust/define optimized treatment.

The invention is suitable for detecting any mutation in EGFR. The term “mutation” designates any alteration in the nucleotide sequence encoding EGFR, such as nucleotide substitution(s), deletion(s), or insertion(s). In a particular embodiment, the mutation is a single nucleotide substitution in a coding (exon) region of the gene. In another particular embodiment, the mutation is a deletion or insertion of one or several (typically from 1 to 15) contiguous nucleotides, typically in an exon of the gene sequence.

The invention utilizes particular sets of allele-specific primers and oligoblockers allowing specific and sensitive detection of mutations in the gene sequence from circulating cell-free nucleic acids. The inventors have selected optimized target sequences within the EGFR gene and optimized primer design, which allow controlled and specific amplification from cell free nucleic acids. The invention can be implemented from any fluid sample containing cfNAs, e.g., cell free DNA (cfDNA), cell free RNA, cell free siRNA and/or cell free miRNA. Cell free nucleic acids are typically circulating nucleic acids contained in biological fluids, so that the method is advantageous as compared to an analysis from tissue biopsy. The invention may be implemented with samples from any subject, including any human patient having or suspected to have cancer.

The method of the invention typically comprises amplifying, in a sample of a biological fluid from the subject containing cell free nucleic acids, at least a first target sequence from the EGFR gene, the first target sequence containing a site of a mutation on said gene, wherein the first amplified target sequence is 50-95 nt in length and amplification of the first target sequence is conducted with a first set of reagents comprising (i) a mutation-specific primer of 15-25 nt in length, (ii) a paired primer of 15-25 nt in length and (iii) an oligoblocker which hybridizes specifically to the first target sequence in non-mutated form resulting in a blockade of polymerase elongation.

The invention may be conducted with any fluid sample containing cfNA, such as blood, plasma, serum, pleural fluid, saliva, urine, or the like. The sample may be pretreated prior to being used in the invention (e.g., diluted, concentrated, separated, partially purified, frozen, etc.). The method is typically performed on a sample of, or derived from, blood, serum or plasma, such as a pretreated blood sample.

The invention uses a first set of primers which is designed to amplify a target sequence of 50-95 nt in length within the EGFR gene, said sequence carrying a site of a targeted mutation in the gene.

The term “primer” designates, within the context of the present invention, nucleic acid molecules that can hybridize specifically to a target genetic sequence and serve to initiate amplification. Primers of the invention are typically single-stranded nucleic acids, with a length advantageously comprised between 10 and 30 bases, preferably between 15 and 30 bases, even more preferably between 15 and 25 bases. Primers should perfectly match with the targeted sequence in the EGFR gene, allowing specific hybridization thereto and essentially no hybridization to another region.

Preferred primers of the invention shall have a sequence with a GC content between 40 and 60%. The GC content is the number of G's and C's in the primer as a percentage of the total bases. Such a level of GC content provides the optimized combination of specific amplification and efficacy. Also, in a set of reagents of the invention, all of the primers shall have a similar Tm. More particularly, the Tm difference between the mutation-specific primer and its paired WT primer should preferably not be more than 5° C. Indeed, with such low difference, the difference of hybridization of each partner primer is low and does not affect specific mutant amplification. More preferably, the Tm of the primers should be between 40-60° C. and should not differ between mutation-specific and paired by more than 5° C. Furthermore, in a preferred embodiment, the primers of the invention should not be self-annealing. Indeed, self-annealing may decrease the quantity of primer available for the amplification and reduce efficacy. Moreover, it contributes to non-specific amplification. The risk of self-annealing and self-hairpin loops of primers may be determined in silico using available software such as the software Oligoanalyzer®, which analyzes the change in Gibbs free energy required for the breaking of secondary structures (ΔG). Typically, if this energy is negative, it should not be below −4 kcal/mol, otherwise risk of self-annealing or self-hairpin loops could occur. In a particular embodiment, primers of the invention shall thus have a ΔG superior or equal to −4 kcal/mol as determined with Oligoanalyzer®. When using online tools as IDT OligoAnalyzer, which rely on different algorithms than Oligoanalyzer® for analyzing the change in Gibbs free energy required for the breaking of secondary structures, the ΔG should not be below −7 kcal/mol otherwise risk of self-annealing or self-hairpin loops could occur. Accordingly, in another particular embodiment, primers of the invention shall have a ΔG superior or equal to −7 kcal/mol as determined with IDT OligoAnalyzer.

As indicated, the invention uses a first set of reagents comprising (i) a mutation-specific primer, (ii) a paired primer and (iii) an oligoblocker.

The mutation-specific primer is designed to hybridize to the mutated sequence, if and when present. In this regard, when the mutation is a nucleotide substitution, the mutation-specific primer preferably contains the mutated position at one terminal nucleotide thereof. Indeed, the invention shows such conformation allows increased specificity of the method. In a preferred embodiment, the mutated position is at the 3′-terminal nucleotide of the mutation-specific primer, or at the 5′-terminal nucleotide of the mutation-specific primer.

In another embodiment, when the mutation is a deletion, the mutation-specific primer overlaps with the junction region in the gene created by said deletion. The primer sequence is preferably centered on the targeted junction.

In still another embodiment, when the mutation is an insertion, the mutation-specific primer overlaps with at least one junction region in the gene created by said insertion. The primer sequence is preferably centered on the targeted junction.

The second primer, the “paired” primer, hybridizes to a sequence in the EGFR gene which is non-mutated and amplifies, with its paired mutation-specific primer, a target sequence of 50-95 nt in length.

The first set of reagent contains an oligoblocker, which increases specificity of the method. The oligoblocker is an oligonucleotide which hybridizes specifically to the first target sequence in non-mutated form, such hybridization resulting in a blockade of amplification. When the targeted mutation is present, the oligoblocker does not hybridize and amplification of the mutated sequence occurs. In contrast, when the mutation is not present, the oligoblocker hybridizes to the target sequence and prevents non-specific amplification. The oligoblocker hybridizes “specifically” to the first target sequence in non-mutated form, which means that its sequence is exactly complementary to the non-mutated sequence. As a result, the oligoblocker essentially cannot hybridizes to the gene when the mutation is present. The oligoblocker preferably has a length similar to that of the two primers. It is thus preferably between 15-25 nucleotides in length. Furthermore, in order to increase specificity, in case of a single nucleotide mutation, the site of the non-mutated nucleotide targeted by the oligoblocker shall be preferably centered on the oligoblocker sequence, more preferably located within 80% central nucleotides, even more preferably within 60% central nucleotides, even further preferably within 40% central nucleotides.

Furthermore, in all embodiments, the oligoblocker is modified, preferably terminally, to prevent amplification upon hybridization, e.g. to prevent polymerase elongation. As a result, when the mutation is not present, the oligoblocker hybridizes to the target sequence and prevents any non-specific amplification. In a preferred embodiment, the oligoblocker is phosphorylated in 3′, resulting in a blockade of polymerase elongation.

Amplification using the above set of reagents from a sample containing cfNA allows specific production of a 50-95 nt long amplicon sequence containing a mutation when the targeted mutation is present, and no production of such an amplicon when the mutation is not present. The results contained in the present application show that such design provides high specificity and high sensitivity with any sample containing cell-free nucleic acids. Detection of an amplification product thus indicates the presence of the mutation in the sample.

Amplification may be performed using any technique known per se in the art such as with a polymerase, e.g., by PCR, Q-PCR, droplet-digital PCR and crystal-digital PCR.

In a preferred embodiment, the method uses two sets of reagents to amplify two distinct target sequences in the EGFR gene: The first set, as described above, targets a sequence containing a (site of a) mutation; and the second set targets a wild-type (“WT”) sequence. By combining the two sets of reagents, the method of the invention allows measuring not only the presence or amount of a mutation, but also the level of total cfNA, thereby providing with high reliability a mutant allele frequency in circulating nucleic acids. The second targeted sequence should be located at a distance of about 100-400 bp of the mutant sequence. Furthermore, the second amplified sequence should have a length comprised between 50-95 nt and similar to that of the first amplified sequence.

In a preferred embodiment, the method thus comprises a step of amplifying a second non-mutated target sequence on the EGFR gene, said second target sequence being located at a distance of 100 to 400 bp from the first target sequence on said gene, the first and second amplified sequences each being 50-95 nt in length and of a similar length.

The term “similar length” indicates that the two amplified sequences should not differ in length by more than 15%, even more preferably by not more than 10%, further preferably by not more than 5%. In other words, if one set amplifies a sequence of 80 nt, the other shall amplify a sequence of 68-92 nt, preferably of 72-88 nt, more preferably of 76-84 nt.

The distance between the two targeted sequence shall be comprised between 100-400 pb. Such a distance designates typically the number of nucleotides contained between the first amplified nucleotide of the first target sequence and the first amplified nucleotide of the second target sequence. Preferably, the distance is comprised between 200-400, more preferably between 250 and 350. Further preferably, at a distance of 300 nucleotides±10%.

The second set of reagents contains a pair of amplification primers, but no oligoblocker. Indeed, the second amplified sequence is non-mutated. The primers in the second set shall have a length between 15-25 nt, and shall preferably have the characteristics as defined above for the first set in terms of Tm, GC content and self-annealing.

Both amplifications are preferably conducted in parallel, by separately incubating aliquots of a test sample with the two sets of reagents and suitable enzymes and nucleotides to perform amplification. The reactions thus produce:

one mutation-specific amplicon of 50-95 nt produced by the first set of reagents (if the mutation is present); and

one WT amplicon of 50-95 nt, produced by the second set of reagents.

Detection of the mutation-specific amplicon indicates the presence of the mutation in the sample. Detection of the other amplicon allows determination of a frequency of mutation in circulating NAs of the subject, e.g., by determining a ratio mt/WT. As illustrated in the experimental section, the method is highly selective and very sensitive.

Alternatively, the two amplifications may be conducted simultaneously, by incubating an aliquot of a test sample with the two sets of reagents and suitable enzymes and nucleotides to perform amplification. Such a reaction produces the following amplicons:

one mutation-specific amplicon of 50-95 nt produced by the first set of reagents (if the mutation is present);

one WT amplicon of 50-95 nt, produced by the second set of reagents; and

one amplicon of 200-400 nt, produced by the combination of primers of the two sets of reagents.

Furthermore, the method may be used to detect several different mutations in the EGFR gene from a subject sample, either sequentially, or in parallel. In such a case, the method uses one set of mutation-specific reagents as defined above for each targeted mutation. Typically, the method also uses at least one set of reagents that amplify a WT target sequence located 100-400 bp from one of the targeted mutated sequence.

Most preferred mutations that are detected with the present invention are described in the following table:

| | | | | | R/S TKI |
|---|---|---|---|---|---|
| Gene | Disease | EXON | Mutation | Frequency | EGFR |
| EGFR | NSCLC | global frequency EGFR mutant: 29% | | | |
| | | (COSMIC 2015) | | | |
| | | frequency of each mutation in EGFR | | | |
| | | mutant tumors | | | |
| | | 18 | G719A | 0.60% | S |
| | | | G719C | 0.30% | S |
| | | | G719S | 0.50% | S |
| | | 19 | exon 19 | 48% | S |
| | | | DEL | | |
| | | 20 | S768I | <1% | S |
| | | 20 | exon 20 INS | 4-9.2% | R |
| | | 20 | T790M | <5% untreated tumors | R |
| | | | | >50% of acquired | |
| | | | | resistance to TKIs | |
| | | 21 | L861Q | 2% | S |
| | | 21 | L858R | 43% | S |

These mutations are correlated to cancer, particularly lung cancer, such as NSCLC. The detection of these mutations, alone or in combinations, allows reliable diagnosis or prognosis of cancer in a subject, and also allows staging of the cancer.

In a particular embodiment, the invention is for detecting T790M mutation. EGFR T790M is a substitution mutation with a high frequency rate, especially in patients having acquired resistance to cancer treatment with tyrosine kinase inhibitors. Detecting the presence of this mutation allows an adaptation of the treatment. The T790M mutation results in an amino acid substitution at position 790, from a threonine (T) to a methionine (M). This mutation occurs within exon 20, which encodes part of the kinase domain. Nomenclature of EGFR T790M point mutation is c: 2369 C>T. It means that the 2369^{th} nucleotide on the cDNA reference sequence is a Cytosine that is substituted by a Thymine.

In a preferred embodiment, the mutation is EGFR T790M and the first set of reagents comprises a mutation-specific primer selected from SEQ ID NO: 1 or 2 (T790M 16; T790M 17), a paired primer selected from SEQ ID NO: 3 or 4 (T790 P19; T790 P20), and an oligoblocker selected from SEQ ID NO: 5 (T790 OB19). Most preferably, the mutation-specific primer is SEQ ID NO: 1 (T790M 16), the paired primer is SEQ ID NO: 3 (T790 P19) and the oligoblocker is SEQ ID NO: 5 (T790 OB19). In a preferred embodiment, the method amplifies a second WT target sequence, located preferably 200-400 bp from the first mutated target sequence, using a second set of reagents. In a more preferred embodiment, the second set of reagents comprises a first primer SEQ ID NO: 6 (T790 WT L19) and a second primer SEQ ID NO: 7 (T790 WT R19).

In another embodiment, the mutation is EGFR L858R and the first set of reagents for detecting the EGFR L858R mutation comprises a mutation-specific primer selected from any one of SEQ ID NOs: 8-11 (L858R 16, L858R 17, L858R 18, L858R 19), a paired primer selected from SEQ ID NO: 12 (L858 P20), and an oligoblocker selected from any one of SEQ ID NOs: 13-14 (L858 OB19/3, L858 OB20/3). Most preferably, the mutation-specific primer is SEQ ID NO: 11 (L858R 19), the paired primer is SEQ ID NO: 12 (L858 P20) and the oligoblocker is SEQ ID NO: 13 (L858 OB19/3). In a preferred embodiment, the method amplifies a second WT target sequence, located preferably 200-400 bp from the first mutated target sequence, using a second set of reagents. In a more preferred embodiment, the second set of reagents comprises a first primer SEQ ID NO: 15 (L858 WT L) and a second primer SEQ ID NO: 16 (L858 WT R).

In another embodiment, the mutation is EGFR L861Q and the first set of reagents for detecting the EGFR L861Q mutation comprises a mutation-specific primer selected from any one of SEQ ID NOs: 17-18 (L861Q i17, L861Q i18), a paired primer selected from SEQ ID NO: 19 (L861 iP20), and an oligoblocker selected from any one of SEQ ID NOs: 20-21 (L861 iOB19/2, L861 iOB20/2). Most preferably, the mutation-specific primer is SEQ ID NO: 18 (L861Q i18), the paired primer is SEQ ID NO: 19 (L861 iP20), and the oligoblocker is SEQ ID NO: 21 (L861 iOB20/2). In a preferred embodiment, the method amplifies a second WT target sequence, located preferably 200-400 bp from the first mutated target sequence, using a second set of reagents. In a more preferred embodiment, the second set of reagents comprises a first primer SEQ ID NO: 22 (L861 WT L) and a second primer SEQ ID NO: 23 (L861 WT R).

In another embodiment, the mutation is EGFR G719S and the first set of reagents for detecting EGFR G719S mutation comprises a mutation-specific primer selected from any one of SEQ ID NOs: 24-25 (G719S 19, G719S 20), a paired primer selected from SEQ ID NO: 26 (G719 P19), and an oligoblocker selected from any one of SEQ ID NOs: 27-28 (G719 OB18/2, G719 OB19/2). Most preferably, the mutation-specific primer is SEQ ID NO: 25 (G719S 20), the paired primer is SEQ ID NO:26 (G719 P19), and the oligoblocker is SEQ ID NO: 28 (G719 OB19/2). In a preferred embodiment, the method amplifies a second WT target sequence, located preferably 200-400 bp from the first mutated target sequence, using a second set of reagents. In a more preferred embodiment, the second set of reagents comprises a first primer SEQ ID NO:29 (G719 WT L) and a second primer SEQ ID NO: 30 (G719 WT R).

In another embodiment, the mutation is EGFR G719A and the first set of reagents for detecting the EGFR G719A mutation comprises a mutation-specific primer selected from any one of SEQ ID NOs: 31-33 (G719A 18, G719A 19, G719A 20), a paired primer selected from SEQ ID NO: 26 (G719 P19), and an oligoblocker selected from any one of SEQ ID NOs: 27-28 (G719 OB18/2, G719 OB19/2). Most preferably, the mutation-specific primer is SEQ ID NO: 31 (G719A 18), the paired primer is SEQ ID NO: 26 (G719 P19) and the oligoblocker is SEQ ID NO: 27 (G719 OB18/2). In a preferred embodiment, the method amplifies a second WT target sequence, located preferably 200-400 bp from the first mutated target sequence, using a second set of reagents. In a more preferred embodiment, the second set of reagents comprises a first primer SEQ ID NO: 29 (G719 WT L) and a second primer SEQ ID NO: 30 (G719 WT R).

In another embodiment, the mutation is EGFR S768I and the first set of reagents for detecting the EGFR S768I mutation comprises a mutation-specific primer selected from any one of SEQ ID NOs: 34-35 (S768I i16, S768I i17), a paired primer selected from SEQ ID NO: 36 (S768 iP18), and an oligoblocker selected from SEQ ID NO: 37 (S768 iOB19). Most preferably, the mutation-specific primer is SEQ ID NO: 35 (S768I i17), the paired primer is SEQ ID NO: 36 (S768 iP18) and the oligoblocker is SEQ ID NO: 37 (S768 iOB19). In a preferred embodiment, the method amplifies a second WT target sequence, located preferably 200-400 bp from the first mutated target sequence, using a second set of reagents. In a more preferred embodiment, the second set of reagents comprises a first primer SEQ ID NO:38 (S768 WT L) and a second primer SEQ ID NO: 39 (S768 WT R).

In another embodiment, the mutation is EGFR exon19 Δ(E746-A750) and wherein the mutation-specific primer is SEQ ID NO: 40 (DEL19 17N), the paired primer is SEQ ID NO: 41 (DEL19 P19), and the oligoblocker is SEQ ID NO: 42 (DEL19 OB21/3). In a preferred embodiment, the method amplifies a second WT target sequence, located preferably 200-400 bp from the first mutated target sequence, using a second set of reagents. In a more preferred embodiment, the second set of reagents comprises a first primer SEQ ID NO: 43 (DEL19 WT L) and a second primer SEQ ID NO: 44 (DEL19 WT R).

In another embodiment, the mutation is EGFR V769-D770 insASV (Ins20) and the first set of reagents for detecting the EGFR Ins20 mutation comprises a mutation-specific primer selected from any one of SEQ ID NOs: 45-49 (INS20 AVS i16, INS20 AVS i16/2, INS20 AVS i16/3, INS20 AVS i16/4, INS20 AVS i16/5), a paired primer selected from any one of SEQ ID NOs: 50-52 (Ins20 iP18, Ins20 iP17, Ins20 iP17/2), and an oligoblocker selected from SEQ ID NO: 53 (Ins20AVS iOB18). Most preferably, the mutation-specific primer is SEQ ID NO: 45 (INS20 AVS i16), the paired primer is SEQ ID NO: 51 (Ins20 iP17) and the oligoblocker is SEQ ID NO: 53 (Ins20AVS iOB18). In a preferred embodiment, the method amplifies a second WT target sequence, located preferably 200-400 bp from the first mutated target sequence, using a second set of reagents. In a more preferred embodiment, the second set of reagents comprises a first primer SEQ ID NO:54 (Ins20 WT L) and a second primer SEQ ID NO: 55 (Ins20 WT R).

In another particular embodiment, the invention is for detecting a combination of two or more mutations in the EGFR gene.

In this regard, in a particular embodiment, the method is for detecting at least two nucleotide substitutions, preferably at least T790M and L858R.

In another embodiment, the method is for detecting at least one nucleotide substitution and one deletion.

In a particular embodiment, the method is for detecting T790M, L858R and DelExon19 (such as exon19 Δ(E746-A750)).

Such combination of mutations is particularly indicative of a subject condition. In particular, the detection of L858R and of deletions of Exon 19 covers approximately 91% of EGFR positive cases where targeted therapy with first-generation TKIs is predicted to work effectively. Moreover, the addition of T790M enables to identify the 5% of treatment-naïve patients T790M-positive that would be resistant to first-generation TKIs, for whom third-generation TKIs should be considered as favored therapeutic option.

### Kits

In a further aspect, the invention relates to kits suitable for detecting EGFR mutations in a sample of a biological fluid containing cell free nucleic acids from a subject. Kits generally comprise reagents, containers and, optionally, instructions to perform the method. The reagents may be in a same container, in discrete parts thereof, of in separate containers. The user may then use any portion of the reagents to perform the reaction, typically following the instructions.

In a more particular embodiment, the kits of the invention comprise at least a first set of reagents (a), wherein the first set of reagents amplifies a first target sequence from the EGFR gene, said first target sequence containing a site of a mutation in the EGFR gene, said first set of reagents comprising (i) a mutation-specific primer of 15-25 nt in length, (ii) a paired primer of 15-25 nt in length positioned such that the amplified first target sequence is 50-95 nt in length, and (iii) an oligoblocker, wherein the oligoblocker hybridizes specifically with the first target sequence in non-mutated form and blocks amplification.

In a further particular embodiment, the kits of the invention further comprise a second set of reagents (b) which amplifies a second non-mutated target sequence from the EGFR gene, said second target sequence being located between 100 and 400 bp from the first target sequence on said gene, and wherein said amplified second target sequence is 50-95 nt in length and of a length similar to that of the first amplified target sequence.

In a further particular embodiment, the kits of the invention comprise at least a third set of reagents (c), wherein the third set of reagents amplifies a third target sequence from the EGFR gene, said third target sequence containing a site of a mutation in the EGFR gene distinct from the first mutation targeted by set of reagent (a), said third set of reagents comprising (i) a mutation-specific primer of 15-25 nt in length, (ii) a paired primer of 15-25 nt in length positioned such that the amplified third target sequence is 50-95 nt in length, and (iii) an oligoblocker, wherein the oligoblocker hybridizes specifically with the third target sequence in non-mutated form and blocks amplification.

Sets of reagents (a), (b) and (c) are preferably in separate containers of the kit, allowing amplification in parallel on different aliquots of a test sample.

In a particular embodiment, the kit further comprises other reagents to perform an amplification reaction, such as an enzyme (e.g., a polymerase such as Taq), nucleotides, and/or a buffer. Such reagents may be in separate containers, to be mixed by the user.

The kit is preferably suitable to detect a mutation in EGFR gene selected from G719A, G719C, G719S, S768I, T790M, L858R, L861Q, exon19DEL and exon20INS.

In a preferred embodiment, the kit of the invention is suitable for detecting the mutation EGFR T790M and the first set of reagents comprises a mutation-specific primer selected from any one of SEQ ID NOs: 1-2 (T790M 16; T790M 17), a paired primer selected from SEQ ID NO: 3 or 4 (T790 P19; T790 P20), and an oligoblocker selected from SEQ ID NO:5 (T790 OB19). Most preferably, the mutation-specific primer is SEQ ID NO: 1 (T790M 16), the paired primer is SEQ ID NO: 3 (T790 P19) and the oligoblocker is SEQ ID NO: 5 (T790 OB19). In a preferred embodiment, the method amplifies a second WT target sequence, located preferably 200-400 bp from the first mutated target sequence, using a second set of reagents. In a more preferred embodiment, the second set of reagents comprises a first primer SEQ ID NO: 6 (T790 WT L19) and a second primer SEQ ID NO: 7 (T790 WT R19).

In another embodiment, the kit of the invention is suitable for detecting the mutation EGFR L858R and the first set of reagents for detecting the EGFR L858R mutation comprises a mutation-specific primer selected from any one of SEQ ID NOs: 8-11 (L858R 16, L858R 17, L858R 18, L858R 19), a paired primer selected from SEQ ID NO: 12 (L858 P20), and an oligoblocker selected from any one of SEQ ID NOs: 13-14 (L858 OB19/3, L858 OB20/3). Most preferably, the mutation-specific primer is SEQ ID NO: 11 (L858R 19), the paired primer is SEQ ID NO: 12 (L858 P20) and the oligoblocker is SEQ ID NO: 13 (L858 OB19/3). In a preferred embodiment, the method amplifies a second WT target sequence, located preferably 200-400 bp from the first mutated target sequence, using a second set of reagents. In a more preferred embodiment, the second set of reagents comprises a first primer SEQ ID NO: 15 (L858 WT L) and a second primer SEQ ID NO: 16 (L858 WT R).

In another embodiment, the kit of the invention is suitable for detecting the mutation EGFR L861Q and the first set of reagents for detecting the EGFR L861Q mutation comprises a mutation-specific primer selected from any one of SEQ ID NOs: 17-18 (L861Q i17, L861Q i18), a paired primer selected from SEQ ID NO: 19 (L861 iP20), and an oligoblocker selected from any one of SEQ ID NOs: 20-21 (L861 iOB19/2, L861 iOB20/2). Most preferably, the mutation-specific primer is SEQ ID NO: 18 (L861Q i18), the paired primer is SEQ ID NO: 19 (L861 iP20), and the oligoblocker is SEQ ID NO: 21 (L861 iOB20/2). In a preferred embodiment, the method amplifies a second WT target sequence, located preferably 200-400 bp from the first mutated target sequence, using a second set of reagents. In a more preferred embodiment, the second set of reagents comprises a first primer SEQ ID NO: 22 (L861 WT L) and a second primer SEQ ID NO: 23 (L861 WT R).

In another embodiment, the kit of the invention is suitable for detecting the mutation EGFR G719S and the first set of reagents for detecting EGFR G719S mutation comprises a mutation-specific primer selected from any one of SEQ ID NOs: 24-25 (G719S 19, G719S 20), a paired primer from SEQ ID NO: 26 (G719 P19), and an oligoblocker selected from any one of SEQ ID NOs: 27-28 (G719 OB18/2, G719 OB19/2). Most preferably, the mutation-specific primer is SEQ ID NO: 25 (G719S 20), the paired primer is SEQ ID NO: 26 (G719 P19), and the oligoblocker is SEQ ID NO: 28 (G719 OB19/2). In a preferred embodiment, the method amplifies a second WT target sequence, located preferably 200-400 bp from the first mutated target sequence, using a second set of reagents. In a more preferred embodiment, the second set of reagents comprises a first primer SEQ ID NO: 29 (G719 WT L) and a second primer SEQ ID NO: 30 (G719 WT R).

In another embodiment, the kit of the invention is suitable for detecting the mutation EGFR G719A and the first set of reagents for detecting the EGFR G719A mutation comprises a mutation-specific primer selected from any one of SEQ ID NOs: 31-33 (G719A 18, G719A 19, G719A 20), a paired primer from SEQ ID NO: 26 (G719 P19), and an oligoblocker selected from any one of SEQ ID NOs: 27-28 (G719 OB18/2, G719 OB19/2). Most preferably, the mutation-specific primer is SEQ ID NO: 31 (G719A 18), the paired primer is SEQ ID NO: 26 (G719 P19) and the oligoblocker is SEQ ID NO: 27 (G719 OB18/2). In a preferred embodiment, the method amplifies a second WT target sequence, located preferably 200-400 bp from the first mutated target sequence, using a second set of reagents. In a more preferred embodiment, the second set of reagents comprises a first primer SEQ ID NO: 29 (G719 WT L) and a second primer SEQ ID NO: 30 (G719 WT R).

In another embodiment, the kit of the invention is suitable for detecting the mutation EGFR S768I and the first set of reagents for detecting the EGFR S768I mutation comprises a mutation-specific primer selected from any one of SEQ ID NOs: 34-35 (S768I i16, S768I i17), a paired primer from SEQ ID NO: 36 (S768 iP18), and an oligoblocker from SEQ ID NO: 37 (S768 iOB19). Most preferably, the mutation-specific primer is SEQ ID NO: 35 (S768I i17), the paired primer is SEQ ID NO: 36 (S768 iP18) and the oligoblocker is SEQ ID NO: 37 (S768 iOB19). In a preferred embodiment, the method amplifies a second WT target sequence, located preferably 200-400 bp from the first mutated target sequence, using a second set of reagents. In a more preferred embodiment, the second set of reagents comprises a first primer SEQ ID NO: 38 (S768 WT L) and a second primer SEQ ID NO: 39 (S768 WT R).

In another embodiment, the kit of the invention is suitable for detecting the mutation EGFR exon19 Δ(E746-A750) and wherein the mutation-specific primer is SEQ ID NO: 40 (DEL19 17N), the paired primer is SEQ ID NO: 41 (DEL19 P19), and the oligoblocker is SEQ ID NO: 42 (DEL19 OB21/3). In a preferred embodiment, the method amplifies a second WT target sequence, located preferably 200-400 bp from the first mutated target sequence, using a second set of reagents. In a more preferred embodiment, the second set of reagents comprises a first primer SEQ ID NO: 43 (DEL19 WT L) and a second primer SEQ ID NO: 44 (DEL19 WT R).

In another embodiment, the kit of the invention is suitable for detecting the mutation EGFR V769-D770 insASV (Ins20) and the first set of reagents comprises a mutation-specific primer selected from any one of SEQ ID NOs: 45-49 (INS20 AVS i16, INS20 AVS i16/2, INS20 AVS i16/3, INS20 AVS i16/4, INS20 AVS i16/5), a paired primer selected from any one of SEQ ID NO:50-52 (Ins20 iP18, Ins20 iP17, Ins20 iP17/2), and an oligoblocker selected from SEQ ID NO: 53 (Ins20AVS iOB18). Most preferably, the mutation-specific primer is SEQ ID NO: 45 (INS20 AVS i16), the paired primer is SEQ ID NO: 51 (Ins20 iP17) and the oligoblocker is SEQ ID NO: 53 (Ins20AVS iOB18). In a preferred embodiment, the method amplifies a second WT target sequence, located preferably 200-400 bp from the first mutated target sequence, using a second set of reagents. In a more preferred embodiment, the second set of reagents comprises a first primer SEQ ID NO: 54 (Ins20 WT L) and a second primer SEQ ID NO: 55 (Ins20 WT R).

The kits of the invention may comprise reagents for detecting one mutation in the EGFR gene.

Alternatively, kits of the invention may comprise reagents suitable for detecting several distinct mutations in the EGFR gene. In this regard, particular preferred kits of the invention comprise reagents as described above for detecting at least T790M and L858R. A particular kit of the invention comprises reagents as described above for detecting T790M and L858R and Exon19del (in particular exon19 Δ(E746-A750)). Such reagents are preferably in separate containers of the kit, allowing amplification in parallel on different aliquots of a test sample.

In this regard, a particular kit of the invention comprises:

a first set of reagents (a) comprising a mutation-specific primer selected from any one of SEQ ID NOs: 1-2 (T790M 16; T790M 17), a paired primer selected from any one of SEQ ID NOs: 3 or 4 (T790 P19; T790 P20), and an oligoblocker selected from SEQ ID NO:5 (T790 OB 19). Most preferably, the mutation-specific primer is SEQ ID NO: 1 (T790M 16), the paired primer is SEQ ID NO: 3 (T790 P19) and the oligoblocker is SEQ ID NO: 5 (T790 OB19);

a second set of reagents (b) for detecting a WT target sequence, located preferably 200-400 bp from the mutated sequence detected by the first set of reagents. In a more preferred embodiment, the second set of reagents comprises a first primer SEQ ID NO: 6 (T790 WT L19) and a second primer SEQ ID NO: 7(T790 WT R19);

a third set of reagents (c) for detecting the EGFR L858R mutation comprising a mutation-specific primer selected from any one of SEQ ID NOs: 8-11 (L858R 16, L858R 17, L858R 18, L858R 19), a paired primer from SEQ ID NO: 12 (L858 P20), and an oligoblocker selected from any one of SEQ ID NOs: 13-14 (L858 OB19/3, L858 OB20/3). Most preferably, the mutation-specific primer is SEQ ID NO: 11 (L858R 19), the paired primer is SEQ ID NO: 12 (L858 P20) and the oligoblocker is SEQ ID NO: 13 (L858 OB 1 9/3).

In a particular embodiment, the kit further comprises a fourth set of reagents (d) for detecting the mutation EGFR exon19 Δ(E746-A750) and wherein the mutation-specific primer is SEQ ID NO: 40 (DEL19 17N), the paired primer is SEQ ID NO: 41 (DEL19 P19), and the oligoblocker is SEQ ID NO: 42 (DEL19 OB21/3).

The invention also relates to the use of kits as described above for detecting EGFR mutations in a sample from a subject, preferably a human subject.

The invention also relates to a nucleic acid molecule consisting of a nucleotide sequence selected from any one of SEQ ID NOs: 1 to 55. Preferred nucleic acid molecules are selected from 1-5, 8-14, 17-21, 24-28, 31-33, 34-37, 40-42 and 45-53. Particular nucleic acid molecules of the invention are mutation-specific primers consisting of a nucleotide sequence selected from any one of SEQ ID Nos: 1,2,8,11,17,18,24,25,31-33,34,35,40 and 45-49.

The invention also relates to the use of a nucleic acid molecule as defined above for in vitro amplification of a target sequence in an EGFR gene.

The invention also relates to a method for diagnosing a patient, comprising detecting a mutation in the EGFR gene in a sample from the patient using a method or kit or nucleic acid as defined above.

The invention also relates to a method for treating a patient, comprising detecting a mutation in the EGFR gene in a sample from the patient using a method or kit or nucleic acid as defined above, and treated subjects having cancer.

Further aspects and advantages of the invention will be disclosed in the following experimental section.

### EXAMPLES

### I. Methods

This section presents a summary of optimal criteria followed to ensure optimal efficiency and sensitivity for all mutation detection systems herein described, in combination with absence of any non-specificity.

I1) Design of the Allele-Specific Primer Targeting the Mutation of Interest

To optimize specificity and sensitivity of the allele-specific primer targeting the single nucleotide mutation of interest, the mutant base was generally positioned as 3′ end last nucleotide. The 3′ end of an allele-specific primer is mismatched for one allele (the WT allele) and is a perfect match for the other allele (the mutated allele). The 3′ positioning of the mismatch has the effect of partially blocking the amplification of the WT allele. Indeed, the Taq polymerase enzyme typically used for DNA amplification preferably extends DNA in presence of a perfect match at the 3′ primer end with a free hydroxyl group present. Furthermore, any residual non-specific amplification from the WT allele is further avoided using an oligoblocker, as discussed in section I3.

The positioning of the targeted mutation as the 3′ end of the allele-specific primers generates two possible options for assay design: one in “conventional configuration”, with the primer sequence matching the reference sequence on the positive strand (hence with the primer annealing to the negative strand) with the mutation in 3′; one in “inverse configuration”, with the primer sequence matching the negative strand (hence with the primer annealing to the positive strand) with the mutation in 3′.

Both options were considered in the experimental section, and the thermodynamic characteristics of both options were generally compared, to ensure optimal Tm and GC % with absence of primer-dimers, blocker-primer dimers, self-dimerization, and hairpins.

In addition, different combinations of primers at different lengths were tested, to provide optimal results.

In the selection of the primers targeting the mutation of interest the following recommendations were followed to prevent the risk of non-specific amplification and to improve the method's sensitivity:
Low melting temperature (Low Tm): Ideally, the Tm of the primer should be 10° C. below the annealing temperature of the PCR system (60° C.), with accepted range between 45-55° C. As the variant is targeted at the 3′end last base of the primer, Tm changes towards the optimal temperature are obtained by varying the primer length at the 5′ end.
Length of the allele-specific primer: The primer targeting the mutation should have low Tm (45-55° C.), suggesting a short length but it should be long enough to be highly specific and provide efficient amplification. Length was found optimal between 16 and 22 bp.
GC %: The GC content (percentage of guanines and cytosines out of the total number of bases) of the primer should ideally fall between 40 and 60%. Lower the GC %, lower the risk of non-specific amplification but it is associated with lower amplification efficiency. Higher the GC %, higher the risk of non-specific amplification but the amplification efficiency is increased.
3′-tail GC %: Higher values (>25%) allow more specific annealing of the primer to its target sequence.
Secondary structures: the formation of primer self-dimers or hairpin loops is detrimental to the PCR efficiency as it considerably decreases the concentration of primer molecules available for the PCR reaction. Also, secondary structures can generate strong background signals that can negatively impact on the quantification accuracy of the desired PCR product. The software Oligoanalyzer® was used to analyze the change in Gibbs free energy required for breaking down the predicted secondary structures (ΔG). ΔG below −4 kcal/mol are indicative of primers at a very strong risk of self-annealing that should hence be modified to reach ΔG values closer to zero or discarded. Absence of any hairpin is ideal. In presence of hairpins with ΔG below −3 kcal/mol the primer should be modified to reach ΔG values closer to zero or discarded.

I2) Paired Wild-Type Primer to the Allele-Specific Primer

In the selection of the paired wild-type primer the following criteria were followed to prevent the risk of non-specific amplification and to improve the method's sensitivity:
Amplicon size: the paired WT primer was positioned at 60-100 bp from the primer targeting the mutation in order to produce an amplicon of 60-100 bp. Such size was indeed found optimal by the inventors for detecting tumour-derived circulating DNA.
DNA strand to target:
   In the conventional configuration (allele-specific primer designed upon the reference sequence, annealing to the negative strand), the paired wild-type primer sequence is the complement-reverse of the reference sequence, in order to anneal to the positive strand.
   In the inverse configuration (allele-specific primer designed as complement-reverse of the reference sequence to maintain the mutation in 3′, annealing to the positive strand), the paired wild type sequence is designed upon the reference sequence, in order to anneal to the negative strand.
Melting temperature: Ideally, the Tm difference between the Tm of the primer targeting the mutation and its paired WT primer should not exceed 5° C. As a consequence, the Tm of the paired WT primer should preferably be 50-60° C.
GC %, 3′ tail GC % and secondary structures: the same parameters described in section I1. were followed.
Off-target amplification: Co-amplification of any secondary product other than the target sequence should be avoided to ensure specificity. Presence of off-target amplification would generate false negative results and compromise the validity of the proposed method. To prevent this eventuality, the selected WT paired primers, together with their matched allele-specific primers, were analyzed using several methods to confirm specificity for optimal primer combinations.
Unwanted homology regions: The amplicons are typically analyzed using the “Blat” online tool on the UCSC Genome Browser website to determine the presence of potential secondary regions of homology that might generate non-specific products or alter the PCR efficiency.

I3) Oligoblocker Design

The oligoblocker is an oligonucleotide complementary to the WT allele with respect to the mutation of interest. It is modified (e.g., phosphorylated in 3′) to block the polymerase elongation. The role of the oligoblocker is to avoid non-specific amplification of the WT sequence at the mutation locus. When the oligoblocker is hybridized to the WT sequence it avoids non-specific hybridization of the primer targeting the mutation to the WT sequence, as it partially overlaps the primer target sequence.

The criteria for an optimal oligoblocker design are herein described, to prevent the risk of non-specific amplification and to improve the detection of low-frequency mutations.
Melting temperature: The oligoblocker Tm should preferably be close to 60° C. (the hybridization/extension temperature of the polymerase enzyme required for the PCR amplification reaction) and preferably at least 4° C. above the Tm of the primer targeting the mutation. This preferred strategy ensures that the oligoblocker hybridizes to its target sequence before any potential non-specific hybridization to the WT locus by the allele-specific primer. Ideally, the oligoblocker Tm should hence be 55-60° C.
Position: The oligoblocker should be designed preferably so as to have the variant nt position towards the middle of its sequence. With this strategy, the oligoblocker occupies a larger portion of the allele-specific primer target region, aiding to avoid non-specific hybridization.
GC % and 3′GC %: the same parameters described in section I.1. should preferably be followed.
Secondary structures: The same parameters described in section I.1 should preferably be followed.
Length: The ideal length of an oligoblocker should be comprised between 19-23 bp.

I4) Combinatorial Hetero-Dimer Analysis

The absence of hetero-annealing among the oligonucleotides (allele-specific primer, paired WT primer and oligoblocker) should preferably be verified. This strategy prevents the risk of forming primer-dimers and non-specific amplification, improving the efficiency of the method. The software Oligoanalyzer® can be used for this purpose and, based on its algorithms for the calculations of Gibbs free energy, just values above −4 Kcal/mol can be accepted. Oligonucleotide combinations with ΔG<−4 kcal/mol need to be discarded and re-designed.

I5) Design of the Wild-Type Primer Pair

This primer set should target a nucleic acid sequence located 100-350 bp, typically 300±10 bases pair, from the nucleic acid sequence targeted by the allele-specific primer set. Moreover, it should preferably target a nucleic acid sequence of the same size than the one targeted by the allele-specific primer set±10%. Preferred characteristics of the primers that ensure optimal performance are listed below:
Melting temperature: The melting temperature should be comprised between 55 and 60° C., with less than 5° C. difference between the Tm of the two primers.
All other thermodynamic parameters to consider in the design of the wild-type primer pair (GC %, 3′GC %, amplicon size, secondary structures, off-target amplification and unwanted homology regions) are the same as described in section I.2.

I6) In Vitro Validation for Mutated Region
Specificity test: This phase is generally performed for verifying that the mutation-specific primer set under evaluation amplifies only its target sequence. It is typically realized on genomic DNA from cell lines carrying the mutation of interest, from commercial DNA carrying the mutation of interest or from synthetic double-strand DNA fragments carrying the mutation of interest diluted in wild-type genomic DNA background.
Non-specificity test: The non-specificity test can be performed to ensure that the primer set targeting the mutation of interest does not amplify any other region and that no primers-dimers or self-dimers are formed during the PCR reaction. Criteria to validate the non-specificity are typically: concentration values below 0.5 pg/μl and a different Tm from the expected Tm of the mutation, as determined by the specificity test.
Efficiency test: The efficiency of mutation-specific primer set is generally evaluated by running the assay on positive controls of known mutational load.
Sensitivity: To assess the sensitivity of the system in analysis, an assay is generally run on serial dilutions of DNA mutant for the variant in analysis.

### II) T790M Mutation

EGFR T790M is a substitution mutation. The T790M mutation results in an amino acid substitution at position 790, from a threonine (T) to a methionine (M). This mutation occurs within EGFR exon 20, which encodes part of the kinase domain. Nomenclature of EGFR T790M point mutation is c: 2369 C>T. It means that the 2369^{th} nucleotide on the cDNA reference sequence is a Cytosine that is substituted by a Thymine.

The inventors have designed specific sets of primers allowing improved detection of the presence (or absence, or frequency) of such a mutation from a sample containing cfNA.

II1) Design of the Allele-Specific T790M Primer

Various allele-specific primers were considered in the design of this invention and their thermodynamic characteristics were analyzed, as shown in Table 2:

TABLE 2
List of all the T790M allele-specific primers
considered in the design of this invention and
their thermodynamic characteristics. Accepted
values are shown with ″*″. Borderline values are
presented with light grey background and non-
acceptable values are shown with ″#″. In the
eventuality that one of the parameters in
analysis falls outside the accepted range
indicated in the last row the thermodynamic
analysis is discontinued and the primer
is discarded.

| | | | | | | | |
|---|---|---|---|---|---|---|---|
| Primer | | | Tm | | GC% | self- | Hair- |
| name | Sequence | Length | (° C.) | GC% | tail | dimers | pins |

Conventional configuration

| | | | | | | | |
|---|---|---|---|---|---|---|---|
| T790M | CCTCCACC | 22 | 70^{#} | ^{#} | ^{#} | ^{#} | ^{#} |
| 22 | GTGCAGCTC | | | | | | |
| | ATCAT | | | | | | |
| | (SEQ ID | | | | | | |
| | NO: 56) | | | | | | |
| T790M | CTCCACCG | 21 | 66^{#} | ^{#} | ^{#} | ^{#} | ^{#} |
| 21 | TGCAGCTC | | | | | | |
| | ATCAT | | | | | | |
| | (SEQ ID | | | | | | |
| | NO: 57) | | | | | | |
| T790M | TCCACCGT | 20 | 62^{#} | ^{#} | ^{#} | ^{#} | ^{#} |
| 20 | GCAGCTCAT | | | | | | |
| | CAT | | | | | | |
| | (SEQ ID | | | | | | |
| | NO: 58) | | | | | | |
| T790M | CCACCGTG | 19 | 60^{#} | ^{#} | ^{#} | ^{#} | ^{#} |
| 19 | CAGCTCAT | | | | | | |
| | CAT | | | | | | |
| | (SEQ ID | | | | | | |
| | NO: 59) | | | | | | |
| T790M | CACCGTGC | 18 | 56^{#} | ^{#} | ^{#} | ^{#} | ^{#} |
| 18 | AGCTCAT | | | | | | |
| | CAT | | | | | | |
| | (SEQ ID | | | | | | |
| | NO: 60) | | | | | | |
| T790M | ACCGTGCA | 17 | 52* | 52.9* | 28.6* | −3.84* | none* |
| 17 | GCTCATCAT | | | | | | |
| | (SEQ ID | | | | | | |
| | NO: 2) | | | | | | |
| T790M | CCGTGCAG | 16 | 50* | 56.3* | 28.6* | −3.84* | none* |
| 16 | CTCATCAT | | | | | | |
| | (SEQ ID | | | | | | |
| | NO: 1 | | | | | | |

Inverse configuration

| | | | | | | | |
|---|---|---|---|---|---|---|---|
| T790M | CAGCCGAA | 22 | 72^{#} | ^{#} | ^{#} | ^{#} | ^{#} |
| i22 | GGGCATGA | | | | | | |
| | GCTGCA | | | | | | |
| | (SEQ ID | | | | | | |
| | NO: 61) | | | | | | |
| T790M | AGCCGAAG | 21 | 68^{#} | ^{#} | ^{#} | ^{#} | ^{#} |
| i21 | GGCATGAG | | | | | | |
| | CTGCA | | | | | | |
| | (SEQ ID | | | | | | |
| | NO: 62) | | | | | | |
| T790M | GCCGAAGG | 20 | 66^{#} | ^{#} | ^{#} | ^{#} | ^{#} |
| i20 | GCATGAGC | | | | | | |
| | TGCA | | | | | | |
| | (SEQ ID | | | | | | |
| | NO: 63) | | | | | | |
| T790M | CCGAAGGG | 19 | 62^{#} | ^{#} | ^{#} | ^{#} | ^{#} |
| i19 | CATGAGC | | | | | | |
| | TGCA | | | | | | |
| | (SEQ ID | | | | | | |
| | NO: 64) | | | | | | |
| T790M | CGAAGGGC | 18 | 58^{#} | ^{#} | ^{#} | ^{#} | ^{#} |
| i18 | ATGAGCTG | | | | | | |
| | CA | | | | | | |
| | (SEQ ID | | | | | | |
| | NO: 65) | | | | | | |
| T790M | GAAGGGCA | 17 | 54* | 58.8* | 57.1* | −3.84* | −1. |
| i17 | TGAGCTGCA | | | | | | 86* |
| | (SEQ ID | | | | | | |
| | NO: 66) | | | | | | |
| T790M | AAGGGCAT | 16 | 50* | 56.3* | 57.1* | −3.84* | −1. |
| i16 | GAGCTGCA | | | | | | 86* |
| | (SEQ ID | | | | | | |
| | NO: 67) | | | | | | |

| | | | | | | |
|---|---|---|---|---|---|---|
| Accepted | 16-22 | 45-55 | 40- | >25% | >−4 | >−3 |
| values for the | bp | ° C. | 60% | | Kcal/ | Kcal/ |
| parameters | | | | | mol | mol |
| in analysis | | | | | | |
| (allele- | | | | | | |
| specific | | | | | | |
| primer shown | | | | | | |
| in bold) | | | | | | |

II2) Paired Wild-Type Primer

Results Obtained with Primer 3

All allele-specific primers that met our criteria (see Table 2) were used on Primer3 to find a compatible paired primer. Results are summarized in Table 3.

TABLE 3
List of all the T790 paired WT- primers produced by Primer 3 and
considered in the design of this invention and their thermodynamic characteristics.

| | Allele- | | | | | | | | | | | Off |
|---|---|---|---|---|---|---|---|---|---|---|---|---|
| | specific | Paired | Paired | | | | | | | | | target |
| Allele- | Primer | WT | WT | | | | | | | | | with wt |
| specific | Tm | primer | primer | | Amplicon | Tm | | GC % | self- | Hair- | Off | allele- |
| Primer | (Primer3) | name | sequence | Length | size | (° C.) | GC % | tail | dimers | pins | target? | specific? |
| T90M | 50.37 | T790 | AGCAG | 20 | 92^{#} | 54.89^{#} | 55^{#} | 42.9^{#} | −1.81^{#} | −1.69^{#} | no^{#} | no^{#} |
| 17 | | P20/ | GTACT | | | | | | | | | |
| | | 92 bp | GGGAG | | | | | | | | | |
| | | | CCAAT | | | | | | | | | |
| | | | (SEQ ID | | | | | | | | | |
| | | | NO: 68) | | | | | | | | | |
| | | T790 | AGCAG | 19 | 92^{#} | 54.56^{#} | 57.89^{#} | 57.1^{#} | −1.81^{#} | −1.69^{#} | no^{#} | no^{#} |
| | | P19/ | GTACT | | | | | | | | | |
| | | 92 bp | GGGAG | | | | | | | | | |
| | | | CCAA | | | | | | | | | |
| | | | (SEQ ID | | | | | | | | | |
| | | | NO: 69) | | | | | | | | | |
| | | T790 | GTTGA | 20 | 96^{#} | 54.61^{#} | 60^{#} | 71.40^{#} | −0.43^{#} | −0.42^{#} | no^{#} | no^{#} |
| | | P20/ | GAGGT | | | | | | | | | |
| | | 96 bp | ACTGG | | | | | | | | | |
| | | | GAGC | | | | | | | | | |
| | | | (SEQ ID | | | | | | | | | |
| | | | NO: 70) | | | | | | | | | |
| | | T790 | CCAGT | 20 | 99^{#} | 54.21^{#} | 60^{#} | 57.1^{#} | −4.7^{§} | −4.6^{§} | ^{§} | ^{§} |
| | | P20/ | TGAGC | | | | | | | | | |
| | | 99 bp | AGGTA | | | | | | | | | |
| | | | CTGGG | | | | | | | | | |
| | | | (SEQ ID | | | | | | | | | |
| | | | NO: 71) | | | | | | | | | |
| T790M | 48.24 | T790 | CTTTG | 20 | 66* | 50.45* | 50* | 42.9* | −6.54^{§} | −0.04^{§} | ^{§} | ^{§} |
| 16 | | P20/ | TGTTC | | | | | | | | | |
| | | 66 bp | CCGGA | | | | | | | | | |
| | | | CATAG | | | | | | | | | |
| | | | (SEQ ID | | | | | | | | | |
| | | | NO: 72) | | | | | | | | | |
| | | T790 | CACCA | 19 | 100^{#} | 50.9^{#} | 52.63^{#} | 42.9^{#} | −1.2^{#} | −1.08^{#} | no^{#} | no^{#} |
| | | P19/ | GTTGA | | | | | | | | | |
| | | 100 bp | GCAGG | | | | | | | | | |
| | | | TACT | | | | | | | | | |
| | | | (SEQ ID | | | | | | | | | |
| | | | NO: 73) | | | | | | | | | |
| T790M | No | ^{§} | ^{§} | ^{§} | ^{§} | ^{§} | ^{§} | ^{§} | ^{§} | ^{§} | ^{§} | ^{§} |
| i 17 | results | | | | | | | | | | | |
| | found^{§} | | | | | | | | | | | |
| T790M | No | ^{§} | ^{§} | ^{§} | ^{§} | ^{§} | ^{§} | ^{§} | ^{§} | ^{§} | ^{§} | ^{§} |
| i 16 | results | | | | | | | | | | | |
| | found^{§} | | | | | | | | | | | |

| | | | | | | | | | |
|---|---|---|---|---|---|---|---|---|---|
| Accepted values for the | 16-25 bp | <100 bp | ΔTm | 40-60% | >25% | >−4 | >−3 | no | no |
| parameters in analysis | | | with | | | Kcal/ | Kcal/ | | |
| (paired WT primer) | | | paired | | | mol | mol | | |
| | | | primer | | | | | | |
| | | | <5° C. | | | | | | |

Tm are calculated following the recommended Primer3 algorithm.
Accepted values are shown with “*”.
Borderline values are shown with “^{#}” and non-acceptable values are shown with “^{§}”.
In the eventuality that one of the parameters in analysis falls outside the accepted range indicated in the last row the thermodynamic analysis is discontinued and the primer is discarded.

From the results presented in Table 2 it was not possible to select a definite candidate as paired WT primer for the T790M allele-specific primer. The candidates with the best thermodynamic characteristics produced amplicons considered borderline for low frequency detection of mutations from tumour-derived cfDNA fragments. On the other hand, the primer T790 P20/66 bp in combination with the allele-specific primer T790M 16 produced an amplicon of suitable size but contains a region causing high self-dimerization.

### Manually Generated Primer Pair Candidates in Conventional Configuration

In view of the unsuccessful generation of a paired primer using conventional tools, we designed such primers manually. All primers are listed in Table 4. Their characteristics were analyzed.

TABLE 4
List of all potentialT790 paired WT- primers in conventional configuration
manually generated and considered in the design of this invention and
their thermodynamic characteristics. Tm are calculated using the
Oligoanalyzer ® software. Accepted values are shown with “*” and
non-acceptable values are shown with “#”. In the eventuality that
one of the parameters in analysis falls outside the accepted range
indicated in the last row the thermodynamic analysis is discontinued
and the primer is discarded.

| | | | | | | | | | | | | Off |
|---|---|---|---|---|---|---|---|---|---|---|---|---|
| | Allele- | Paired | Paired | | | | | | | | | target |
| Allele- | specific | WT | WT | | | | | | | | | with wt |
| specific | Primer | primer | primer | | Amplicon | Tm | | GC% | self- | Hair- | Off | allele- |
| Primer | Tm | name | sequence | Length | size | (° C.) | GC% | tail | dimers | pins | target? | specific? |
| T790M | 50 | T790 | ATATTGTCTTT | 20 | 73 | 56^{#} | 40^{#} | 71.4^{#} | −0.7^{#} | none^{#} | no^{#} | no^{#} |
| 16 | | P20/73bp | GTGTTCCCG | | | | | | | | | |
| | | | (SEQ ID NO: 74) | | | | | | | | | |
| | | T790 | TATTGTCTTTG | 19 | 72 | 54* | 42.10* | 71.4* | none* | none* | no* | no* |
| | | P19/72bp | TGTTCCCG | | | | | | | | | |
| | | | (SEQ ID NO: 75) | | | | | | | | | |
| T790M | 52 | T790 | ATATTGTCTTT | 20 | 74 | 56* | 40* | 71.4* | −0.7* | none* | no* | no* |
| 17 | | P20/74bp | GTGTTCCCG | | | | | | | | | |
| | | | (SEQ ID NO: 74) | | | | | | | | | |
| | | T790 | TATTGTCTTTG | 19 | 73 | 54* | 42.10* | 71.4* | none* | none* | no* | no* |
| | | P19/73bp | TGTTCCCG | | | | | | | | | |
| | | | (SEQ ID NO: 75) | | | | | | | | | |
| Accepted values for the | | | | 16-25 | <100bp | ΔTm | 40- | >25% | >−4 | >−3 | no | no |
| parameters in analysis | | | | bp | | with | 60% | | Kcal/ | Kcal/ | | |
| (paired WT primer) | | | | | | paired | | | mol | mol | | |
| | | | | | | primer | | | | | | |
| | | | | | | <5° C. | | | | | | |

The following combinations were thus found, that meet the thermodynamic criteria required:
T790M 16+T790 P19/72 bp
T790M 17+T790 P19/73 bp
T790M 17+T790 P20/74 bp.

### Blat Alignment of the Validated Primer Pairs in Conventional Configuration

Blat alignments (Human GRCh38/hg38) for all the PCR amplicons generated by the combinations of primers meeting the thermodynamic criteria were performed. All primer pairs produced just one single amplicon at the expected chromosomal position. No other homologies were reported.

### Manually Generated Primer Pair Candidates in Inverse Configuration

We further designed paired primers in inverse configuration, as listed in Table 5.

TABLE 5
List of all potential T790 paired WT- primers in inverse configuration manually
generated and considered in the design of this invention and their thermodynamic
characteristics. Tm are calculated using the Oligoanalyzer ® software.
Accepted values are shown with “*”. Borderline values are shown
with “#” and non-acceptable values are shown with “§”.
In the eventuality that one of the parameters in analysis
falls outside the accepted range indicated in the last row the
thermodynamic analysis is discontinued and the primer is discarded.

| | | | | | | | | | | | | Off |
|---|---|---|---|---|---|---|---|---|---|---|---|---|
| | Allele- | Paired | Paired | | Am- | | | | | | | target |
| Allele- | specific | WT | WT | | pli- | | | | | | | with wt |
| specific | Primer | primer | primer | | con | Tm | | GC% | self- | Hair- | Off | allele- |
| Primer | Tm | name | sequence | Length | size | (° C.) | GC% | tail | dimers | pins | target? | specific? |
| T790M | 54 | T790 i | CGTGGACAAC | 20 | 82 | 66^{§} | ^{§} | ^{§} | ^{§} | ^{§} | ^{§} | ^{§} |
| i 17 | | P20/82bp | CCCCACGTGT | | | | | | | | | |
| | | | (SEQ ID | | | | | | | | | |
| | | | NO: 76) | | | | | | | | | |
| | | T790 i | GTGGACAACC | 19 | 81 | 62^{§} | ^{§} | ^{§} | ^{§} | ^{§} | ^{§} | ^{§} |
| | | P19/81bp | CCCACGTGT | | | | | | | | | |
| | | | (SEQ ID | | | | | | | | | |
| | | | NO: 77) | | | | | | | | | |
| | | T790 i | TGGACAACCC | 18 | 80 | 58* | 61.1* | 57.1* | −7^{§} | ^{§} | ^{§} | ^{§} |
| | | P18/80bp | CCACGTGT | | | | | | | | | |
| | | | (SEQ ID | | | | | | | | | |
| | | | NO: 78) | | | | | | | | | |
| | | T790 i | GGACAACCCC | 17 | 79 | 56* | 64.7* | 57.1* | −7^{§} | ^{§} | ^{§} | ^{§} |
| | | P17/79bp | CACGTGT | | | | | | | | | |
| | | | (SEQ ID | | | | | | | | | |
| | | | NO: 79) | | | | | | | | | |
| | | T790 i | GACAACCCCC | 16 | 78 | 52* | 62.5* | 57.1* | −7^{§} | ^{§} | ^{§} | ^{§} |
| | | P16/78bp | ACGTGT | | | | | | | | | |
| | | | (SEQ ID | | | | | | | | | |
| | | | NO: 80) | | | | | | | | | |
| | | T790 i | CGTGGACAAC | 18 | 82 | 60^{§} | ^{§} | ^{§} | ^{§} | ^{§} | ^{§} | ^{§} |
| | | P18/82bp-2 | CCCCACGT | | | | | | | | | |
| | | | (SEQ ID | | | | | | | | | |
| | | | NO: 81) | | | | | | | | | |
| | | T790 i | CGTGGACAAC | 17 | 82 | 58* | 70.6^{#} | 85.7^{#} | −6.67^{§} | ^{§} | ^{§} | ^{§} |
| | | P17/82bp-3 | CCCCACG | | | | | | | | | |
| | | | (SEQ ID | | | | | | | | | |
| | | | NO: 82) | | | | | | | | | |
| | | T790 i | CGTGGACAAC | 16 | 82 | 54* | 68.8^{#} | 85.7^{#} | −3.16* | −3.04^{#} | ^{§} | ^{§} |
| | | P16/82bp-4 | CCCCAC | | | | | | | | | |
| | | | (SEQ ID | | | | | | | | | |
| | | | NO: 83) | | | | | | | | | |
| Accepted values for the | | | | 16-25 | <100bp | ΔTm | 40- | >25% | >−4 | >−3 | no | no |
| parameters in analysis | | | | bp | | with | 60% | | Kcal/ | Kcal/ | | |
| (paired WT primer) | | | | | | paired | | | mol | mol | | |
| | | | | | | primer | | | | | | |
| | | | | | | <5° C. | | | | | | |

As can be seen from Table 5, no suitable combinations could be identified by designing the T790M assay in inverse configuration.

II3) Oligoblocker for the Allele-Specific T790M Assay

We generated several oligoblocker that target the wild-type sequence for T790, and tested their characteristics in combination with the primers.

TABLE 6
List of all oligoblockers for the T790 codon, in
conventional configuration, considered in the
design of this invention and their thermodynamic
characterisitics. Tm are calculated using the
Oligoanalyzer ® software. Accepted values are
shown with “*”. Borderline values are shown with
“#” and non-acceptable values are shown with
“§”. In the eventuality that one of the para-
meters in analysis falls outside the accepted
range indicated in the last row the thermo-
dynamic analysis is discontinued and the
oligoblocker is discarded.

| Oligo- | | | | | | | |
|---|---|---|---|---|---|---|---|
| blocker | | | Tm | | GC% | self- | Hair- |
| name | Sequence | Length | (° C.) | GC% | tail | dimers | pins |
| T790 | GTGCAGCT | 23 | 72^{§} | ^{§} | ^{§} | ^{§} | ^{§} |
| OB23 | CATCACGC | | | | | | |
| | AGCTCAT | | | | | | |
| | (SEQ ID | | | | | | |
| | NO: 84) | | | | | | |
| T790 | TGCAGCT | 22 | 68^{§} | ^{§} | ^{§} | ^{§} | ^{§} |
| OB22 | CATCACG | | | | | | |
| | CAGCTCA | | | | | | |
| | (SEQ ID | | | | | | |
| | NO: 85) | | | | | | |
| T790 | GCAGCTCA | 21 | 66^{§} | ^{§} | ^{§} | ^{§} | ^{§} |
| OB21 | TCACGCAG | | | | | | |
| | CTCAT | | | | | | |
| | (SEQ ID | | | | | | |
| | NO: 86) | | | | | | |
| T790 | CAGCTCA | 20 | 62^{§} | ^{§} | ^{§} | ^{§} | ^{§} |
| OB20 | TCACGCA | | | | | | |
| | GCTCAT | | | | | | |
| | (SEQ ID | | | | | | |
| | NO: 87) | | | | | | |
| T790 | CAGCTCA | 19 | 60* | 57.9* | 57.1* | −3.13* | −3. |
| OB19 | TCACGCA | | | | | | 01^{#} |
| | GCTCA | | | | | | |
| | (SEQ ID | | | | | | |
| | NO: 5) | | | | | | |

| | | | | | | |
|---|---|---|---|---|---|---|
| Accepted | 19- | ~60° | 40- | >25% | >−4 | >−3 |
| values for | 23 bp | C. | 60% | | Kcal/ | Kcal/ |
| the parameters | | | | | Mol | Mol |
| in analysis | | | | | | |
| (Oligoblocker) | | | | | | |

The best candidate was T790 OB 19, despite the possibility of hairpins with borderline values (−3.01 Kcal/mol). The hairpin was predicted to open at a temperature of 43.7° C. Given that the oligoblocker annealing takes place at a temperature of 60° C., the hairpin should not cause any efficiency problem. All further thermodynamic analyses were thus conducted considering T790 OB 19 as best oligoblocker candidate.

II4) Thermodynamic Analysis on All Candidate Primers and Oligoblockers

The following heterodimers combinations were considered for all the primers/oligoblocker candidates that met the thermodynamic criteria described in the previous sections:
Allele-specific primer+paired WT primer;
Allele-specific primer+oligoblocker;
Paired WT primer+oligoblocker.
   The results are presented in Table 7.

TABLE 7
Evaluation of heterodimers presence for all oligonucleotide candidates
evaluated for the allele-specific system.

| | ΔG | Validated? |
|---|---|---|
| Oligonucleotide combination | (Kcal/mol) | Y/N |
| T790M 16 + T790 P19/72bp | none | Y |
| T790M 17 + T790 P19/72bp | none | Y |
| T790M 17 + T790 P20/74bp | none | Y |
| T790M 16 + T790 OB19 | −3.7 | Y |
| T790M 17 + T790 OB19 | −3.7 | Y |
| T790 P19/72bp + T790 OB19 | −0.08 | Y |
| T790 P20/74bp + T790 OB19 | −0.08 | Y |
| Accepted values for the | >−4 Kcal/mol | Y |
| parameters in analysis | | |

The results show all oligonucleotide candidates meet the thermodynamic criteria required to ensure absence of non-specific amplification and to improve the amplification efficiency.

II5) Design of the T790M Wild-Type Primer Set

To produce optimal T790M Wild-Type primer sets, we chose to target the DNA region shown in FIG. 3.

### T790M Wild-Type Set Candidates

The tested primer candidates are disclosed in Table 8.

TABLE 8
Evaluation of all thermodynamic parameters considered to selected
appropriate oligonucleotides for the T790M wild-type set.
Accepted values are shown with an “*”.

| Primer | | | Tm | | GC% | self- | Hair- | Hetero- | Validated |
|---|---|---|---|---|---|---|---|---|---|
| name | Sequence | Length | (° C.) | GC% | tail | dimers | pins | dimers | (Y/N) |
| T790 WT L | CCTGTGCTAG | 19 | 58* | 52.6* | 42.9* | −1.46* | −1.34* | None* | Y |
| | GTCTTTTGC | | | | | | | | |
| | (SEQ ID NO: 6) | | | | | | | | |
| T790 WT R | CATGAATGCG | 19 | 54* | 42.1* | 42.9* | −2.17* | None* | | Y |
| | ATCTTGAGT | | | | | | | | |
| | (SEQ ID NO: 7) | | | | | | | | |

| | | | | | | | | |
|---|---|---|---|---|---|---|---|---|
| Accepted values for | 16- | 55-60° C. | 40- | >25% | >−4 | >−3 | >−4 | Y |
| the parameters in analysis | 22bp | ΔTm | 60% | | Kcal/ | Kcal/ | Kcal/ | |
| (wild-type primer set) | | with | | | mol | mol | mol | |
| | | paired | | | | | | |
| | | primer | | | | | | |
| | | <5° C. | | | | | | |

Our results confirm the candidate primers exhibit the required characteristics. The pair was thus validated for further analysis.

### Blat Alignment of the Validated Primer Pairs in Conventional Sense

The Blat local alignment highlighted the presence of a secondary homology region. Nevertheless, by analyzing the structure and extension of the homology region reported on chr14, we concluded that it would at most produce just a partial annealing region for the T790 WT L primer but, as no homology is present in the target region of the T790 WT R primer, no secondary product would be expected to be produced. This primer combination could thus be validated.

The oligonucleotide candidates of the T790M WT set met the thermodynamic criteria required to ensure absence of non-specific amplification and to improve the amplification efficiency.

II6) In Vitro Validation of the T790M Primer Pairs

### T790M 18+T790 P20+T790 OB19

### Specificity

The specificity of the combination was tested. They showed one peak only, indicating the specificity criteria are met.

### Non-Specificity

A substantial non-specificity was observed when using this oligonucleotide combination. This combination is thus not optimal.

### T790M 18+T790 P19+T790 OB19

### Specificity

The specificity of the combination was tested. The results show one peak only, indicating the specificity criteria are met.

### Non-Specificity

Melting curves of genomic DNA WT for EGFR T790M from the Difi cell line was compared to the melting curves of commercial DNA mutant for EGFR T790M using this combination of oligonucleotides. A substantial non-specificity was observed when using this oligonucleotide combination. This combination is thus not optimal.

### T790M 16+T790 P19+T790 OB19

### Specificity

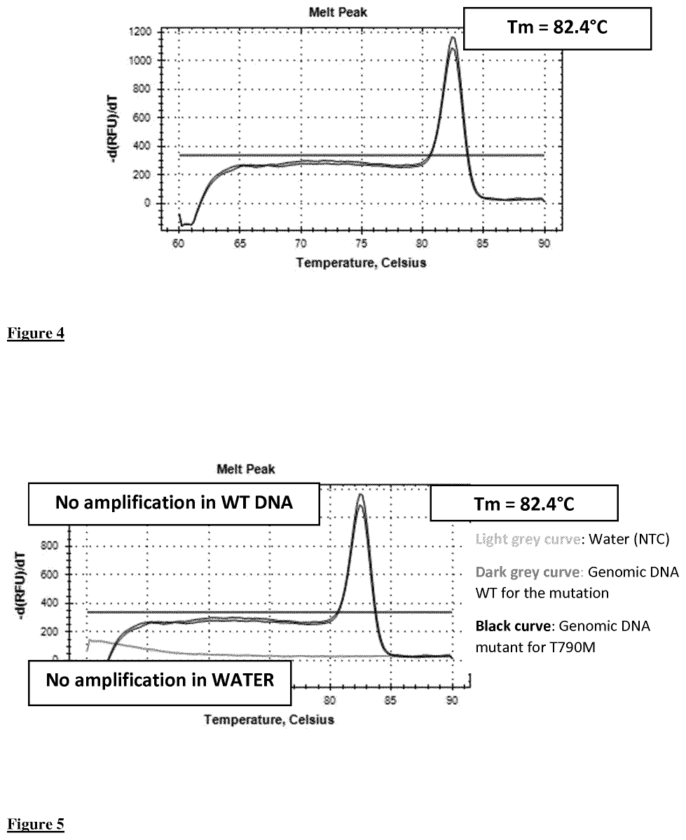

The specificity of the combination was tested. Melting curves of a commercial DNA mutant for EGFR T790M was determined using this oligonucleotide system. Just one peak was observed, as presented in FIG. 4. indicating the specificity criteria are met.

### Non-Specificity

Melting curves of genomic DNA WT for T790M from the Difi cell line was compared to melting curves of commercial DNA mutant for EGFR T790M using this combination of oligonucleotides (see FIG. 5). There is not any non-specificity observed. This oligonucleotide combination was thus further evaluated for efficacy.

### Efficiency

The efficiency analysis for the mutation-specific set comprised of T790M 16+T790 P19+T790 OB19 was performed in conjunction with the proposed combination for the T790 WT primer set. The results are presented in Table 9.

TABLE 9
Demonstration of the efficiency of the oligonucleotide system for the detection of
EGFR T790M. The target T790M corresponds to the combination T790M 16 + T790 P19 +
T790 OB19. The target T790 WT corresponds to the combination T790 WL L + T790 WT R.
The target KRAS corresponds to an internal reference primer pair previously validated with
efficiency close to 100%.

| | | | | [DNA] | | |
|---|---|---|---|---|---|---|
| Target | Content | Sample | Cq | ng/μl | mean | mA % |
| T790M | Positive | T790M | 26.59 | 0.38 | 0.39 | 62.94 |
| | control | 50% | 26.53 | 0.39 | | |
| T790 WT | | Horizon | 25.96 | 0.61 | 0.61 | |
| KRAS (internal | | standard | 26.05 | 0.74 | 0.74 | 52.70% |
| reference) | | | | | | |

The observed mutant allele frequency of the T790M mutation-specific system on the T790 WT system is 62%, close to expected 50% reported by the supplier of the positive control used for the experiment.

The efficiency is thus appropriate and this oligonucleotide combination was further evaluated for sensitivity.

### Sensitivity

The results are presented in Table 10.

TABLE 10
Demonstration of the high sensitivity of the method for the detection of the EGFR T790M
point mutation. Serial dilutions of a commercial DNA control positive for the T790M
mutation at expected mutational load of 0.25 ng/μl were performed, up to 1/10000.

| | | mutant [DNA] | Specific | | Theoretical |
|---|---|---|---|---|---|
| Sample | Cq | (ng/μl) | Tm? | mean | concentration |
| Ctrl+ T790M | 26.54 | 0.3342 | Y | 0.33705 | 0.25 |
| | 26.51 | 0.3399 | Y | | |
| Ctrl+ T790M | 29.35 | 0.0455 | Y | 0.04433 | 0.025 |
| 1/10 | 29.42 | 0.0431 | Y | | |
| Ctrl+ T790M | 31.59 | 0.0092 | Y | 0.0045 | 0.0025 |
| 1/100 | N/A | N/A | Y | | |
| | 31.56 | 0.0095 | Y | | |
| | N/A | N/A | N/A | | |
| | 31.76 | 0.0082 | Y | | |
| | N/A | N/A | N/A | | |
| Ctrl+ T790M | 35.44 | 0.000604 | Y | 0.00010 | 0.00025 |
| 1/1000 | N/A | N/A | N/A | | |
| | N/A | N/A | N/A | | |
| | N/A | N/A | N/A | | |
| | 36.84 | 0.000225 | N | | |
| | N/A | N/A | N/A | | |
| Ctrl+ T790M | N/A | N/A | N/A | | |
| 1/10000 | N/A | N/A | N/A | | |
| | N/A | N/A | N/A | | |
| | N/A | N/A | N/A | | |
| | N/A | N/A | N/A | | |
| | N/A | N/A | N/A | | |

The system could detect the T790M mutation down to a frequency of 0.1 pg/μl. The sensitivity is thus very high.

The results here presented confirmed that the method comprised of the mutation-specific set T790M 16+T790 P19+T790 OB19 and of the wild-type set T790 WL L+T790 WT R exhibits high specificity efficiency and sensitivity.

II7) Comparison with Prior Art Method

We compared the efficiency of our method to the technique described in Zhao et al., Oncology Letters 11:2573:2579, 2016. This system uses a mismatch at the third base of 3′ ends to decrease the non-specific combination of the allele-specific primer with the WT allele.

We employed the Zhao system and compared its sensitivity to our system on mutant genomic DNA and on circulating DNA from clinical samples. Zhao primer system targets a 106 bp mutant sequence whilst the invention targets a mutant sequence of 73 bp.

TABLE 11
Comparison of the efficacy of the two systems for the qualitative and quantitative
detection of the EGFR T790M mutation.

| | | | | | | Invention system | |
|---|---|---|---|---|---|---|---|
| | | Zhao system | | | | Results | |
| | | | [DNA] | | | obtained | |
| Efficiency | | | (ng/μL) | Tm | | by DiaDX | theoretical |
| Target | Sample | Cq | extract | specificity | mean | system | conc. |
| B2/T790M | CQ + T790M | 32.15 | 0.00326 | Y | 0.00358 | 0.34 | 0.5 |
| Bk | | 31.87 | 0.00390 | Y | | | |
| | CQ + T790M | 37.25 | 0.00013 | Y | 0.00013 | 0.044 | 0.05 |
| | 1/10 | 37.1 | 0.00014 | Y | | | |
| | CQ + T790M | N/A | N/A | Y | 0.00029 | 0.009 | 0.005 |
| | 1/100 | 35.94 | 0.00029 | | | | |
| | | N/A | N/A | | | | |
| | | N/A | N/A | | | | |
| | | N/A | N/A | | | | |
| | | N/A | N/A | | | | |
| | CQ + T790 | N/A | N/A | | N/A | 0.0006 | 0.0005 |
| | 1/1000 | N/A | N/A | | | | |
| | | N/A | N/A | | | | |
| | | N/A | N/A | | | | |
| | | N/A | N/A | | | | |
| | | N/A | N/A | | | | |

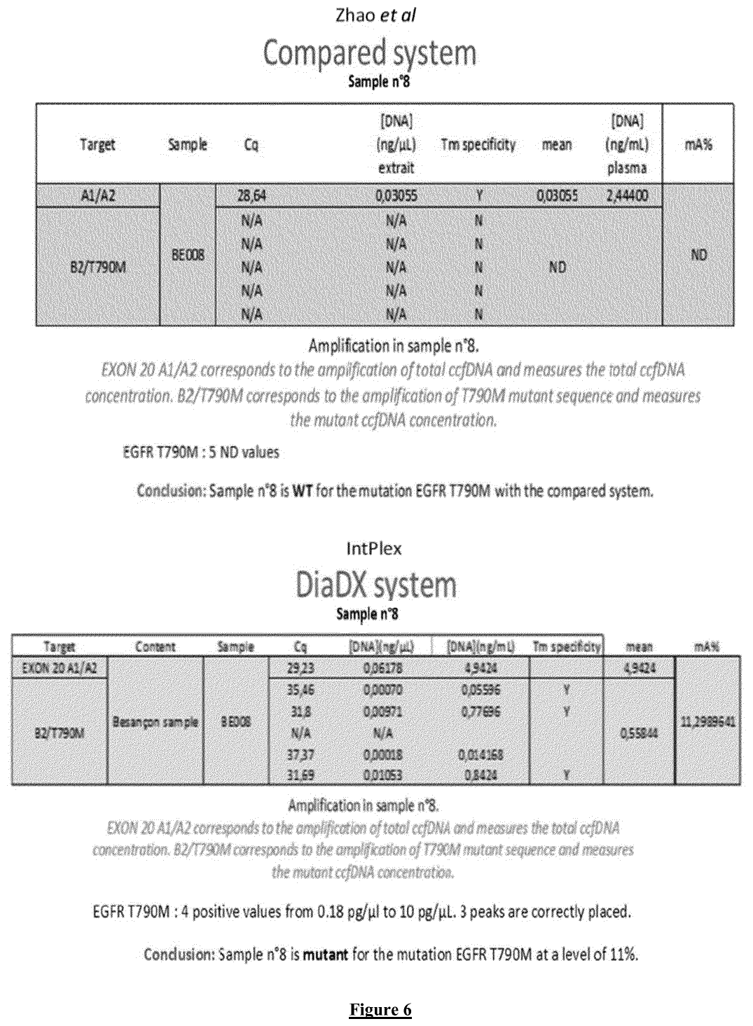

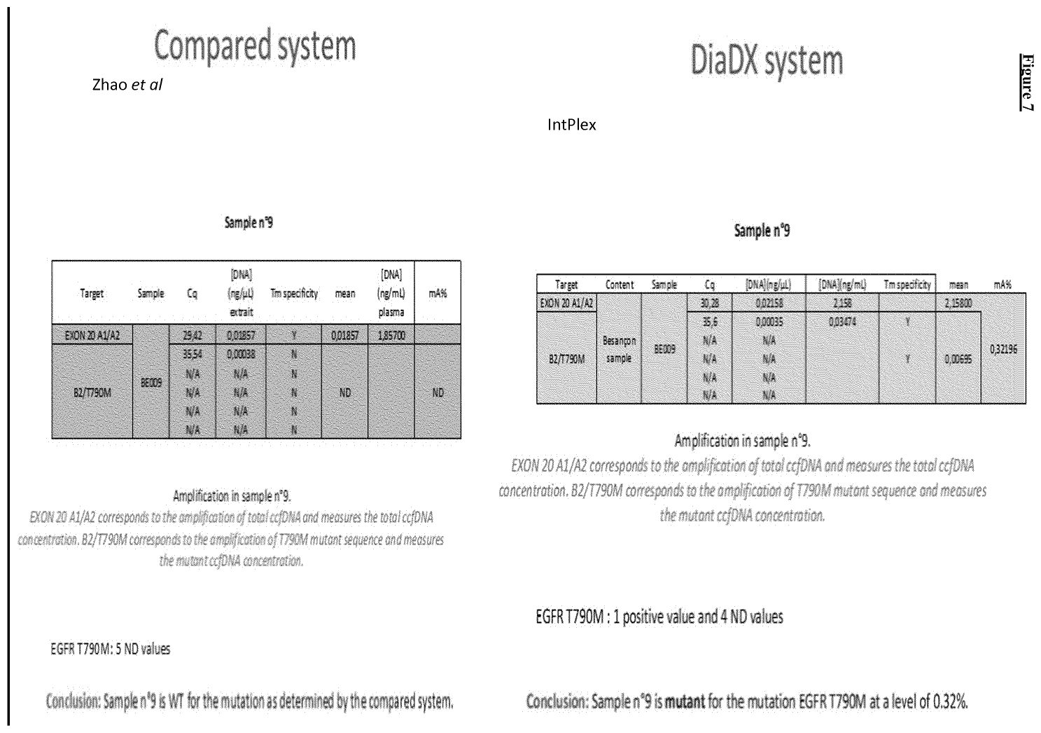

The result obtained show that Zhao system is less efficient than our method, as in same cases it does not detect mutations present at 11% in frequency when analyzing clinical samples from lung cancer patients (see FIG. 6). Zhao system underestimates the real concentrations of mutant DNA and does not allow to determinate concentrations below 0.05 ng/μL (see FIG. 7).

Such results highlight the strong efficacy and sensitivity of our method compared with other mutation detection systems

II8) Conclusions

Our method is specific, sensitive and more efficient than prior art techniques for detecting T790M. Among tested primer combinations, the best system to detect the EGFR T790M mutation is comprised by the mutation specific oligonucleotides: T790M 16+T790 P19+T790 OB19, in combination with the wild-type set T790 WT L+T790 WT R.

Other oligonucleotide combinations that allow efficient detection include:
T790M 17+T790 P19/72 bp+T790 OB19
T790M 17+T790 P20/74 bp+T790 OB19.

### III) L858R Mutation

EGFR L858R is a substitution mutation. The L858R mutation results in an amino acid substitution at position 858, from a Leucine (L) to an Arginine (R). This mutation occurs within exon 21, which encodes part of the kinase domain. Nomenclature of EGFR L858R point mutation is c: 2573 T>G. It means that the 2573^{rd} nucleotide on the cDNA reference sequence is a Thymine that is substituted by a Guanine. After positioning the variant position on the cDNA sequence, it is positioned on the genomic DNA sequence.

The inventors have designed specific sets of primers allowing improved detection of the presence (or absence, or frequency) of such a mutation from a sample containing cfNA.

III1) Design for the Allele-Specific L858R Primer

Various allele-specific primers were considered in the design of this invention and their thermodynamic characteristics were analyzed, as shown in Table 12:

TABLE 12
List of all the EGFR L858R allele-specific
primers considered in the design of this
invention and their thermodynamic character-
istics. Accepted values are shown with “*”.
Borderline values are shown with “#” and non-
acceptable values are shown with “§”. In the
eventuality that one of the parameters in
analysis falls outside the accepted range
indicated in the last row the thermodynamic
analysis is discontinued and the primer is
discarded.

| | | | | | | | |
|---|---|---|---|---|---|---|---|
| Primer | | | Tm | | GC% | self- | Hair- |
| name | Sequence | Length | (° C.) | GC% | tail | dimers | pins |

Conventional configuration

| | | | | | | | |
|---|---|---|---|---|---|---|---|
| L858R | TCAAGA | 22 | 64^{§} | ^{§} | ^{§} | ^{§} | ^{§} |
| 22 | TCACAG | | | | | | |
| | ATTTTG | | | | | | |
| | GGCG | | | | | | |
| | (SEQ ID | | | | | | |
| | NO: 88) | | | | | | |
| L858R | CAAGAT | 21 | 62^{§} | ^{§} | ^{§} | ^{§} | ^{§} |
| 21 | CACAGA | | | | | | |
| | TTTTGG | | | | | | |
| | GCG | | | | | | |
| | (SEQ ID | | | | | | |
| | NO: 89) | | | | | | |
| L858R | AAGATC | 20 | 58^{§} | ^{§} | ^{§} | ^{§} | ^{§} |
| 20 | ACAGAT | | | | | | |
| | TTTGGG | | | | | | |
| | CG | | | | | | |
| | (SEQ ID | | | | | | |
| | NO: 90) | | | | | | |
| L858R | AGATCA | 19 | 56^{#} | 47.4* | 71.4^{#} | -1.41* | 0.28* |
| 19 | CAGATT | | | | | | |
| | TTGGGC | | | | | | |
| | G | | | | | | |
| | (SEQ ID | | | | | | |
| | NO: 11) | | | | | | |
| L858R | GATCAC | 18 | 54* | 50* | 71.4^{#} | -1.41* | 0.28* |
| 18 | AGATTT | | | | | | |
| | TGGGCG | | | | | | |
| | (SEQ ID | | | | | | |
| | NO: 10) | | | | | | |
| L858R | ATCACA | 17 | 50* | 47.1* | 71.4^{#} | 0.16* | none* |
| 17 | GATTTT | | | | | | |
| | GGCG | | | | | | |
| | (SEQ ID | | | | | | |
| | NO: 9) | | | | | | |
| L858R | TCACAG | 16 | 48* | 50* | 71.4^{#} | none* | none* |
| 16 | ATTTT | | | | | | |
| | GGGCG | | | | | | |
| | (SEQ ID | | | | | | |
| | NO: 8) | | | | | | |

Inverse configuration

| | | | | | | | |
|---|---|---|---|---|---|---|---|
| L858R | TCCGCA | 22 | 74^{§} | ^{§} | ^{§} | ^{§} | ^{§} |
| i 22 | CCCAGC | | | | | | |
| | AGTTTG | | | | | | |
| | GCCC | | | | | | |
| | (SEQ ID | | | | | | |
| | NO: 91) | | | | | | |
| L858R | CCGCAC | 21 | 72^{§} | ^{§} | ^{§} | ^{§} | ^{§} |
| i 21 | CCAGCA | | | | | | |
| | GTTTGG | | | | | | |
| | CCC | | | | | | |
| | (SEQ ID | | | | | | |
| | NO: 92) | | | | | | |
| L858R | CGCACC | 20 | 68^{§} | ^{§} | ^{§} | ^{§} | ^{§} |
| i 20 | CAGCAG | | | | | | |
| | TTTGGC | | | | | | |
| | CC | | | | | | |
| | (SEQ ID | | | | | | |
| | NO: 93) | | | | | | |
| L858R | GCACCA | 19 | 64^{§} | ^{§} | ^{§} | ^{§} | ^{§} |
| i 19 | GCAGTT | | | | | | |
| | TGGCCC | | | | | | |
| | (SEQ ID | | | | | | |
| | NO: 94) | | | | | | |
| L858R | CACCCA | 18 | 60^{§} | ^{§} | ^{§} | ^{§} | ^{§} |
| i 18 | GCAGTT | | | | | | |
| | TGGCCC | | | | | | |
| | (SEQ ID | | | | | | |
| | NO: 95) | | | | | | |
| L858R | ACCCAG | 17 | 56^{#} | 64.7^{§} | 71.4^{§} | ^{§} | ^{§} |
| i 17 | CAGTTT | | | | | | |
| | GGCCC | | | | | | |
| | (SEQ ID | | | | | | |
| | NO: 96) | | | | | | |
| L858R | CCCAGC | 16 | 54* | 68.8^{§} | 71.4^{§} | ^{§} | ^{§} |
| i 16 | AGTTTG | | | | | | |
| | GCCC | | | | | | |
| | (SEQ ID | | | | | | |
| | NO: 97) | | | | | | |

| | | | | | | |
|---|---|---|---|---|---|---|
| Accepted | 16-22 | 45-55 | 40- | >25% | >−4 | >−3 |
| values | bp | ° C. | 60% | | Kcal/ | Kcal/ |
| for the | | | | | mol | mol |
| parameters | | | | | | |
| in analysis | | | | | | |
| (allele- | | | | | | |
| specific | | | | | | |
| primer shown | | | | | | |
| in bold) | | | | | | |

This preliminary analysis suggested that primer design in inverse sense is thermodynamically less favorable.

III2) Paired Wild-Type Primer

Results Obtained with Primer 3

All allele-specific primers that met our criteria (see Table 12) were used on Primer3 to find a compatible paired primer. Results are summarized in Table 13.

TABLE 13
List of the EGFR L858 paired WT- primers produced by Primer 3 and
considered in the design of this invention and their thermodynamic
characteristics. Tm are calculated following the recommended
Primer3 algorithm. Accepted values are shown with “*”. Borderline
values are shown with “#” and non-acceptable values are
shown with “§”. In the eventuality that one of the
parameters in analysis falls outside the accepted range indicated
in the last row the thermodynamic analysis is discontinued and the
primer is discarded.

| | Allele- | | | | | | | | | | | Off |
|---|---|---|---|---|---|---|---|---|---|---|---|---|
| | specific | Paired | | | | | | | | | | target |
| Allele- | Primer | WT | Paired WT | | | | | | | | | with wt |
| specific | Tm | primer | primer | | Amplicon | Tm | | GC% | self- | Hair- | Off | allele- |
| Primer | (Primer 3) | name | sequence | Length | size | (° C.) | GC% | tail | dimers | pins | target? | specific? |
| L858R | 58.6 | L858 P20 | CTGACCTAAA | 20 | 94^{#} | 58.72* | 55* | 57.1* | none* | none* | no* | no* |
| 19 | | | GCCACCTCCT | | | | | | | | | |
| | | | (SEQ ID NO: 98) | | | | | | | | | |

| | | | | | | | | | |
|---|---|---|---|---|---|---|---|---|---|
| Accepted values for the parameters | 16-25 | <100bp | ΔTm | 40- | >25% | >−4 | >−3 | no | no |
| in analysis (paired WT primer) | bp | | with | 60% | | Kcal/ | Kcal/ | | |
| | | | paired | | | mol | mol | | |
| | | | primer | | | | | | |
| | | | <5° C. | | | | | | |

The only candidate identified via Primer3 has acceptable thermodynamic characteristics but it produces a PCR amplicon considered borderline for low frequency detection of mutations from tumour-derived cfDNA fragments. It was thus not possible to find appropriate primer using such approach.Manually generated primer pair candidates in conventional configuration.

In view of the unsuccessful generation of a paired primer using conventional tools, we designed such primers manually. All designed primers are listed in Table 14. Their characteristics were analyzed.

TABLE 14
List of a potential L858R paired WT- primers in conventional configuration
manually generated and considered in the design of this
invention and its thermodynamic characteristics.
Tm are calculated using the Oligoanalyzer ® software.
Accepted values are shown with “*” and non-acceptable values are
shown with “#”. In the eventuality that one of the
parameters in analysis falls outside the accepted range indicated
in the last row the thermodynamic analysis is discontinued and the
primer is discarded.

| | | | | | | | | | | | | Off |
|---|---|---|---|---|---|---|---|---|---|---|---|---|
| | Allele- | Paired | | | | | | | | | | target |
| Allele- | specific | WT | Paired WT | | | | | | | | | with wt |
| specific | Primer | primer | primer | | Amplicon | Tm | | GC% | self- | Hair- | Off | allele- |
| Primer | Tm | name | sequence | Length | size | (° C.) | GC% | tail | dimers | pins | target? | specific? |
| L858R | 58.6 | L858 | CTCCTTCTGCA | 20 | 66* | 58* | 45* | 28.6* | -3.84* | none* | no* | no* |
| 19 | | P20/2 | TGGTATTCT | | | | | | | | | |
| | | | (SEQ ID NO: 12) | | | | | | | | | |

| | | | | | | | | | |
|---|---|---|---|---|---|---|---|---|---|
| Accepted values for the parameters | 16-25 | <100bp | ΔTm | 40- | >25% | >−4 | >−3 | no | no |
| in analysis (paired WT primer) | bp | | with | 60% | | Kcal/ | Kcal/ | | |
| | | | paired | | | mol | mol | | |
| | | | primer | | | | | | |
| | | | <5° C. | | | | | | |

The following combinations were found, that meet the thermodynamic criteria required:
L858R 16+L858 P20/2
L858R 17+L858 P20/2
L858R 18+L858 P20/2
L858R 19+L858 P20/2.

### Blat Alignment of the Validated Primer Pairs in Conventional Sense

Blat alignments (Human GRCh38/hg38) for all the PCR amplicons generated by the combinations of primers meeting the thermodynamic criteria were performed. All primer pairs produced just one single amplicon at the expected chromosomal position. No other homologies are reported.

III3) Oligoblocker Design for the EGFR L858R Position

The presence of a region of self-annealing prevents the design of the L858 oligoblocker with the WT base towards the middle of the oligonucleotide sequence. The oligoblocker was designed with the variant position towards its 3′ end. The oligoblockers and their characteristics are described in Table 15.

TABLE 15
List of all potential oligoblockers for the L858
codon with the variant base kept towards the
oligonucleotide 3′ to avoid regions of self-
dimerization, with their thermodynamic
characteristics. All oligoblockers were designed
in conventional configuration. Tm are calculated
using the Oligoanalyzer ® software. Accepted
values are shown with “*”. Borderline values
are shown with “#” and non-acceptable values are
shown with “§”. In the eventuality that one of
the parameters in analysis falls outside the
accepted range indicated in the last row the
thermodynamic analysis is discontinued and the
oligoblocker is discarded.

| Oligo- | | | | | | | |
|---|---|---|---|---|---|---|---|
| blocker | | | Tm | | GC% | self- | Hair- |
| name | Sequence | Length | (° C.) | GC% | tail | dimers | pins |
| L858 | AAGATCACAGA | 23 | 68^{§} | ^{§} | ^{§} | ^{§} | ^{§} |
| OB23/3 | TTTTGGGCTGGC | | | | | | |
| | (SEQ ID NO: 99) | | | | | | |
| L858 | AGATCACAGA | 22 | 66^{§} | ^{§} | ^{§} | ^{§} | ^{§} |
| OB22/3 | TTTTGGGCTGGC | | | | | | |
| | (SEQ ID NO: 100) | | | | | | |
| L858 | GATCACAGATT | 21 | 64^{§} | ^{§} | ^{§} | ^{§} | ^{§} |
| OB21/3 | TTGGGCTGGC | | | | | | |
| | (SEQ ID NO: 101) | | | | | | |
| L858 | ATCACAGATTTT | 20 | 62* | 50* | 85.7^{#} | −0.34* | −0.22* |
| OB20/3 | GGGCTGGC | | | | | | |
| | (SEQ ID NO: 14) | | | | | | |
| L858 | TCACAGATTTTG | 19 | 60* | 52.6* | 85.7^{#} | −0.34* | −0.22* |
| OB19/3 | GGCTGGC | | | | | | |
| | (SEQ ID NO: 13) | | | | | | |

| | | | | | | |
|---|---|---|---|---|---|---|
| Accepted values for | 19- | ~60° | 40- | >25% | >−4 | >−3 |
| the parameters | 23bp | C. | 60% | | Kcal/ | Kcal/ |
| in analysis | | | | | Mol | Mol |
| (Oligoblocker | | | | | | |
| shown in bold) | | | | | | |

Oligoblockers L858 OB20/3 and L858 OB19/3 present the best thermodynamic characteristics.

TABLE 16
Evaluation of suitable combinations of mutation-specific L858R primers in
combination with the best two oligoblockers.

| Mutant primer | | Candidate oligoblocker | |
|---|---|---|---|
| mut-primer name | Tm | L858 OB20/03 Tm = 62 | L858 OB19/03 Tm = 60 |
| L858R 19 | 56 | yes | borderline |
| L858R 18 | 54 | yes | yes |
| L858R 17 | 50 | yes | yes |
| L858R 16 | 48 | yes | yes |

From the Tm analysis of Table 16, all the combinations of allele-specific primers and oligoblocker seem to fit the optimal criteria.

III4) Thermodynamic Analysis on All Candidate Primers and Oligoblockers

The following heterodimers combinations were considered for all the primers/oligoblockers that met the thermodynamic criteria:
Allele-specific primer+paired WT primer
Allele-specific primer+oligoblocker
Paired WT primer+oligoblocker.

The results are presented in Table 17:

TABLE 17
Evaluation of heterodimers presence for all oligonucleotide candidates.

| Oligonucleotide combination | ΔG (Kcal/mol) | Validated? Y/N |
|---|---|---|
| L858R 19 + L858 P20/2 | −1.92 | Y |
| L858R 19 + L858 OB19/3 | −0.34 | Y |
| L858 P20/2 + L858 OB19/3 | −1.92 | Y |
| L858R 18 + L858 P20/2 | −1.92 | Y |
| L858R 18 + L858 OB19/3 | −0.34 | Y |
| L858R 17 + L858 P20/2 | −1.92 | Y |
| L858R 17 + L858 OB19/3 | −0.34 | Y |
| Accepted values for the | >−4 Kcal/mol | Y |
| parameters in analysis | | |

The results show all oligonucleotide candidates meet the thermodynamic criteria required to ensure absence of non-specific amplification and to improve the amplification efficiency.

III5) Design of the L858 Wild-Type Primer Set

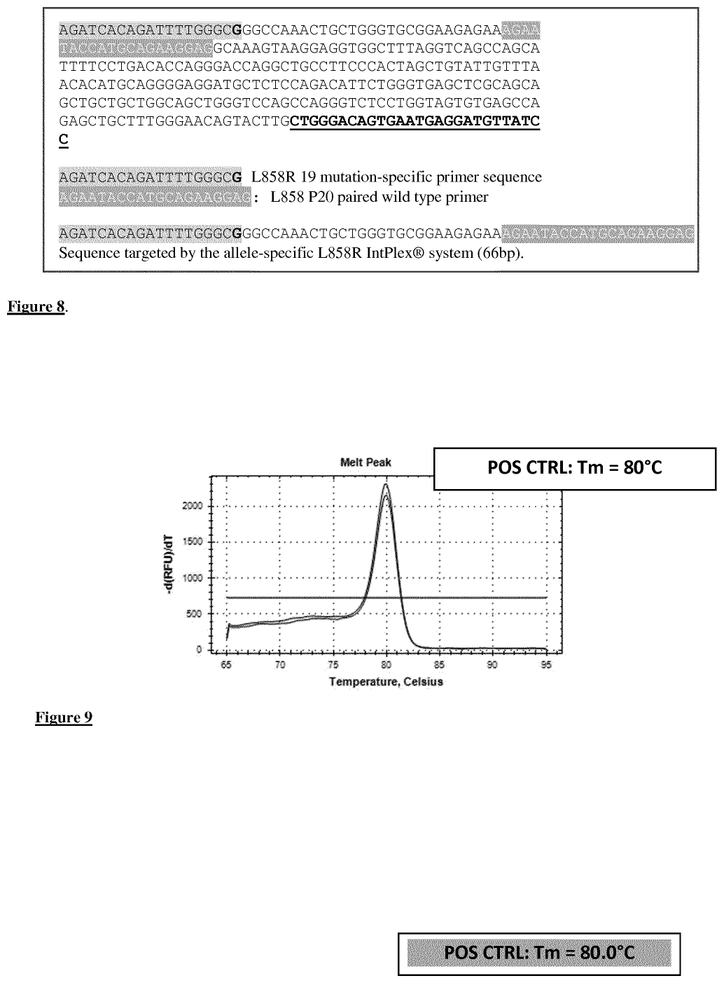

To produce optimal L858R wild-type primer sets, we chose to target the DNA region presented in FIG. 8.

### L858 Wild-Type Set Candidates:

The tested primer candidates are disclosed in Table 18.

TABLE 18
Evaluation of all thermodynamic parameters considered to select appropriate
oligonucleotides for the L858 wild-type set.

| | | | GC % | self- | | | Validated |
|---|---|---|---|---|---|---|---|
| Length | Tm (° C.) | GC % | tail | dimers | Hairpins | Heterodimers | (Y/N) |
| 17 | 54* | 58.8* | 42.9* | none* | none* | −181* | Y |
| 18 | 54* | 50* | 57.1* | none* | none* | | Y |
| 16-22 bp | 54-60° C. | 40- | >25% | >−4 | >−3 | >−4 | Y |
| | ΔTm with | 60% | | | Kcal/ | Kcal/ | Kcal/ |
| | paired | | | | Mol | mol | Mol |
| | primer | | | | | | |
| | <5° C. | | | | | | |

Accepted values are shown with “*”.

Our results confirm the primers satisfy the required optimal characteristics.

### Blat Alignment of the Validated Primer Pairs in Conventional Sense

Blat alignments (Human GRCh38/hg38) for all the PCR amplicons generated by the combinations of primers were performed.

The oligonucleotide candidates of the L858 WT set meet the thermodynamic criteria required to ensure absence of non-specific amplification and to improve the amplification efficiency.

III6) In Vitro Validation of the L858R IntPlex® System

### L858R 19+L858 P20+L858 OB19/3

### Specificity

The specificity of the combination was tested. The results are presented in FIG. 9. They show one peak only, which indicates this oligonucleotide combination meets the specificity criteria.

### Non-Specificity

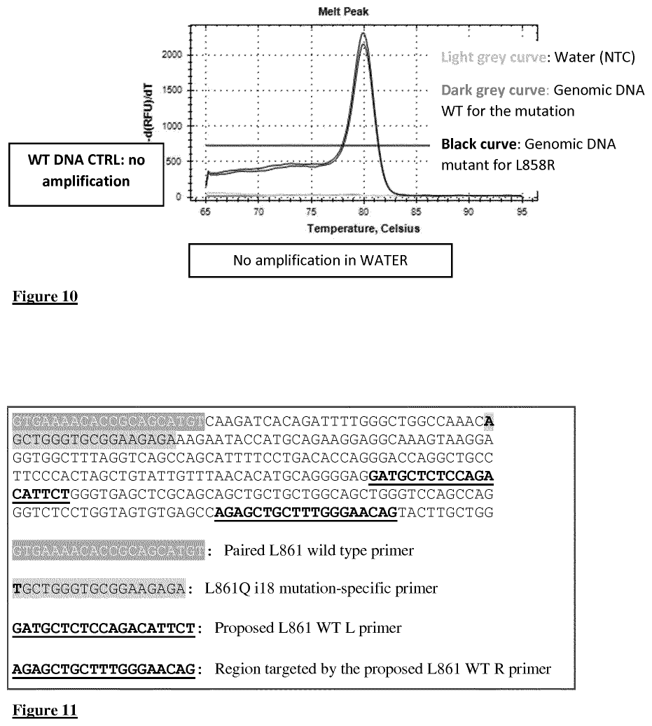

Melting curves of genomic DNA WT for the L858R mutation were compared to melting curves of commercial DNA mutant for L858R using this oligonucleotide combination (see FIG. 10). There was not any non-specificity observed. This oligonucleotide combination was thus evaluated for efficiency.

### Efficiency

The efficiency analysis for the mutation-specific set comprised of L858R 19+L858 P20+L858 OB19 was performed in conjunction with the proposed combination for the L858WT primer set. The results are presented in Table 19.

TABLE 19
Demonstration of the efficiency of the oligonucleotide system in analysis for the
detection of EGFR L858R. The mA % is calculated as ratio between the quantification
performed on a commercial positive control for L858R, with expected frequency of 50%.

| | | | | [DNA] | | |
|---|---|---|---|---|---|---|
| Target | Content | Sample | Cq | ng/μl | Mean | mA % |
| L858R 19 + | Neg control | Di-Fi | N/A | N/A | | |
| L858 P20 + | | DNA | N/A | N/A | | |
| L858 OB19/3 | | | N/A | N/A | | |
| | NTC | Water | N/A | N/A | | |
| | | | N/A | N/A | | |
| | | | N/A | N/A | | |
| | Positive | Horizon | 27.73 | 0.238 | 0.24 | 76.58% |
| | control | std | 27.65 | 0.248 | | |
| L858 WT Assay | | L858R | 27.21 | 0.329 | 0.32 | |
| (L858 WT L + | | 50% | 27.32 | 0.306 | | |
| L858 WT R) | | | | | | |

The observed mutant allele frequency of the L858R mutation-specific system on the L858WT system is 76%, close to expected 50% reported by the supplier of the positive control used for the experiment.

The efficiency is thus appropriate and this oligonucleotide combination was further evaluated for sensitivity.

### Sensitivity

The results are presented in Table 20.

TABLE 20
Demonstration of the high sensitivity of the method for the detection of the EGFR L858R
point mutation. Serial dilutions of a commercial DNA control positive for the L858R
mutation were performed, up to 1/10000.

| | | mutant | | |
|---|---|---|---|---|
| | | [DNA] | Specific | |
| Sample | Cq | (ng/μl) | Tm? | mean |
| Ctrl+ L858R | 28.7 | 0.0722 | Y | 0.07745 |
| | 28.51 | 0.0827 | Y | |
| Ctrl+ L858R 1/10 | 31.05 | 0.0136 | Y | 0.00715 |
| | 35.33 | 0.0007 | Y | |
| Ctrl+ L858R 1/100 | N/A | N/A | Y | 0.0001 |
| | N/A | N/A | Y | |
| | N/A | N/A | Y | |
| | N/A | N/A | N/A | |
| | 35.62 | 0.0005 | Y | |
| | N/A | N/A | N/A | |
| Ctrl+ L858R 1/1000 | N/A | N/A | N/A | |
| | N/A | N/A | N/A | |
| | N/A | N/A | N/A | |
| | N/A | N/A | N/A | |
| | N/A | N/A | N/A | |
| | N/A | N/A | N/A | |
| Ctrl+ L858R 1/10000 | N/A | N/A | N/A | |
| | N/A | N/A | N/A | |
| | N/A | N/A | N/A | |
| | N/A | N/A | N/A | |
| | N/A | N/A | N/A | |
| | N/A | N/A | N/A | |

The system can detect the L858R mutation down to a frequency of 0.1 pg/μl. The sensitivity is thus very high.

The results here presented confirm that the method comprised of the mutation-specific set L858R 19+L858 P20+L858 OB19/3 and of the wild-type set L858 WT L+L858 WT R exhibits high specificity, efficiency and sensitivity.

III7) Conclusion

Our method allows detection of L858R with high specificity, sensitivity and efficiency. Among tested primers, the optimal combination is L858R 19+L858 P20/64 bp+L858 OB19/3. Other suitable primer combinations include:
L858R 19+L858 P20+L858 OB20/3
L858R 18+L858 P20+L858 OB19/3
L858R 18+L858 P20+L858 OB20/3
L858R 17+L858 P20+L858 OB19/3
L858R 17+L858 P20+L858 OB20/3
L858R 16+L858 P20+L858 OB19/3
L858R 16+L858 P20+L858 OB20/3.

### IV) L861Q Mutation

EGFR L861Q is a substitution mutation. The L861Q mutation results in an amino acid substitution at position 861, from a Leucine (L) to a Glutamine (Q). This mutation occurs within exon 21, which encodes part of the kinase domain. Nomenclature of EGFR L861Q point mutation is c: 2582 T>A. It means that the 2582^{th} nucleotide on the cDNA reference sequence is a Thymine that is substituted by an Adenine.

The inventors have designed specific sets of primers allowing improved detection of the presence (or absence, or frequency) of such a mutation from a sample containing cfNA.

IV1) Design of the Allele-Specific EGFR L861Q Primer

Various allele-specific primers were considered in the design of this invention and their thermodynamic characteristics were analyzed, as shown in Table 22.

TABLE 22
List of all the EGFR L861Q allele-specific
primers considered in the design of this
invention and their thermodynamic character-
istics. Accepted values are shown with “*”.
Borderline values are shown with “#” and non-
acceptable values are shown with “§”. In the
eventuality that one of the parameters in
analysis falls outside the accepted range
indicated in the last row the thermodynamic
analysis is discontinued and the primer is
discarded.

| | | | | | | | |
|---|---|---|---|---|---|---|---|
| Primer | | | Tm | | GC% | self- | Hair- |
| name | Sequence | Length | (° C.) | GC% | tail | dimers | pins |

Conventional configuration

| | | | | | | | |
|---|---|---|---|---|---|---|---|
| L861Q | CAGATT | 22 | 66^{§} | ^{§} | ^{§} | ^{§} | ^{§} |
| 22 | TTGGG | | | | | | |
| | CTGGCC | | | | | | |
| | AAACA | | | | | | |
| | (SEQ ID | | | | | | |
| | NO: 102) | | | | | | |
| L861Q | AGATTT | 21 | 62^{§} | ^{§} | ^{§} | ^{§} | ^{§} |
| 21 | TGGGC | | | | | | |
| | TGGCC | | | | | | |
| | AAACA | | | | | | |
| | (SEQ ID | | | | | | |
| | NO: 103) | | | | | | |
| L861Q | GATTT | 20 | 60^{§} | ^{§} | ^{§} | ^{§} | ^{§} |
| 20 | TGGGC | | | | | | |
| | TGGCC | | | | | | |
| | AAACA | | | | | | |
| | (SEQ ID | | | | | | |
| | NO: 104) | | | | | | |
| L861Q | ATTTT | 19 | 56^{§} | ^{§} | ^{§} | ^{§} | ^{§} |
| 19 | GGGCT | | | | | | |
| | GGCC | | | | | | |
| | AAACA | | | | | | |
| | (SEQ ID | | | | | | |
| | NO: 105) | | | | | | |
| L861Q | TTTT | 18 | 54* | 50.3* | 42.9* | -9.98^{§} | ^{§} |
| 18 | GGGCT | | | | | | |
| | GGCC | | | | | | |
| | AAACA | | | | | | |
| | (SEQ ID | | | | | | |
| | NO: 106) | | | | | | |
| L861Q | TTTGGG | 17 | 52* | 52.9* | 42.9* | -9.98^{§} | ^{§} |
| 17 | CTGGCC | | | | | | |
| | AAACA | | | | | | |
| | (SEQ ID | | | | | | |
| | NO: 107) | | | | | | |
| L861Q | TTGGGC | 16 | 50* | 56.3* | 42.9* | -9.98^{§} | ^{§} |
| 16 | TGGCC | | | | | | |
| | AAACA | | | | | | |
| | (SEQ ID | | | | | | |
| | NO: 108) | | | | | | |

Inverse configuration

| | | | | | | | |
|---|---|---|---|---|---|---|---|
| L861Q | TCTTTC | 22 | 68^{§} | ^{§} | ^{§} | ^{§} | ^{§} |
| i 22 | TCTTC | | | | | | |
| | CGCACC | | | | | | |
| | CAGCT | | | | | | |
| | (SEQ ID | | | | | | |
| | NO: 109) | | | | | | |
| L861Q | CTTTCT | 21 | 66^{§} | ^{§} | ^{§} | ^{§} | ^{§} |
| i 21 | CTTCC | | | | | | |
| | GCACC | | | | | | |
| | CAGCT | | | | | | |
| | (SEQ ID | | | | | | |
| | NO: 110) | | | | | | |
| L861Q | TTTCT | 20 | 62^{§} | ^{§} | ^{§} | ^{§} | ^{§} |
| i 20 | CTTCC | | | | | | |
| | GCACC | | | | | | |
| | CAGCT | | | | | | |
| | (SEQ ID | | | | | | |
| | NO: 111) | | | | | | |
| L861Q | TTCTC | 19 | 60^{§} | ^{§} | ^{§} | ^{§} | ^{§} |
| i 19 | TTCC | | | | | | |
| | GCACC | | | | | | |
| | CAGCT | | | | | | |
| | (SEQ ID | | | | | | |
| | NO: 112) | | | | | | |
| L861Q | TCTC | 18 | 58* | 61.1* | 61.1* | -3.13* | none* |
| i 18 | TTCC | | | | | | |
| | GCACC | | | | | | |
| | CAGCT | | | | | | |
| | (SEQ ID | | | | | | |
| | NO: 18) | | | | | | |
| L861Q | CTCTT | 17 | 56* | 64.7* | 71.4* | -3.13* | none* |
| i 17 | CCGC | | | | | | |
| | ACC | | | | | | |
| | CAGCT | | | | | | |
| | (SEQ ID | | | | | | |
| | NO: 17) | | | | | | |
| L861Q | TCTT | 16 | 52* | 62.5* | 71.4* | -3.13* | none* |
| i 16 | CCGCA | | | | | | |
| | CCC | | | | | | |
| | AGCT | | | | | | |
| | (SEQ ID | | | | | | |
| | NO: 113) | | | | | | |

| | | | | | | |
|---|---|---|---|---|---|---|
| Accepted | 16-22 | 45-58 | 40- | >25% | >−4 | >−3 |
| values | bp | ° C. | 60% | | Kcal/ | Kcal/ |
| for the | | | | | mol | mol |
| parameters | | | | | | |
| in analysis | | | | | | |
| (allele- | | | | | | |
| specific | | | | | | |
| primer shown | | | | | | |
| in bold) | | | | | | |

IV2) Paired Wild-Type Primer

Results Obtained with Primer 3

All allele-specific primers that met our IntPlex® criteria (see Table 22) were used on Primer3 to find a compatible paired primer. Unfortunately, no suitable primers were identifiable through this approach.

New potential paired-wild type primer candidates will be manually designed to overcome this issue.

### Manually Generated Primer Pair Candidates in Inverse Configuration

In view of the unsuccessful generation of a paired primer using conventional tools, we designed such primers manually. All primers are listed in Table 23. There characteristics were analyzed.

TABLE 23
Thermodynamic characteristics of one potential L861 paired WT
primers in inverse configuration manually generated and considered
in the design of this invention, together with potential allele-
specific candidates. Tm are calculated using the Oligoanalyzer ®
software. Accepted values are shown with “*” and non-acceptable
values are shown with “#”. In the eventuality that one of the
parameters in analysis falls outside the accepted range indicated
in the last row the thermodynamic analysis is discontinued and the
primer is discarded.

| | | | | | | | | | | | | Off |
|---|---|---|---|---|---|---|---|---|---|---|---|---|
| | Allele- | Paired | | | | | | | | | | target |
| Allele- | specific | WT | Paired WT | | | | | | | | | with wt |
| specific | Primer | primer | primer | | Amplicon | Tm | | GC% | self- | Hair- | Off | allele- |
| Primer | Tm | name | sequence | Length | size | (° C.) | GC% | tail | dimers | pins | target? | specific? |
| L861Q | 58* | L861 | GTGAAAACAC | 20 | 67* | 60* | 50* | 42.9* | -2.17* | 0.04* | No* | No* |
| i 18 | | P20 | CGCAGCATGT | | | | | | | | | |
| L861Q | 56* | | (SEQ ID NO: 19) | | 66* | | | | | | | |
| i 17 | | | | | | | | | | | | |
| L861Q | 52^{#} | | | | 65^{#} | | ^{#} | ^{#} | ^{#} | ^{#} | ^{#} | ^{#} |
| i 16 | | | | | | | | | | | | |

| | | | | | | | | | |
|---|---|---|---|---|---|---|---|---|---|
| Accepted values for the parameters | 16-25 | <100bp | ΔTm | 40- | >25% | >−4 | >−3 | no | no |
| in analysis (paired WT primer) | bp | | with | 60% | | Kcal/ | Kcal/ | | |
| | | | paired | | | mol | mol | | |
| | | | primer | | | | | | |
| | | | <5° C. | | | | | | |

The following combinations were thus found, that meet the thermodynamic criteria required:
L861Q i18+L861 iP20
L861Q i17+L861 iP20.

### Blat Alignment of the Validated Primer Pairs in Conventional Sense

Blat alignments (Human GRCh38/hg38) for all the PCR amplicons generated by the combinations of primers meeting the thermodynamic criteria were performed.

The oligonucleotide candidates of the L861Q set met the thermodynamic criteria required to ensure absence of non-specific amplification and to improve the amplification efficiency.

IV3) Oligoblocker for the Allele-Specific EGFR L861Q Assay

TABLE 24
List of all potential oligoblockers for the EGFR
L861Q codon, in inverse configuration, considered
in the design of this invention and their
thermodynamic characteristics. Tm are calc-
ulated using the Oligoanalyzer ® software.
Accepted values are shown with “*” and non-
acceptable values are shown with “#”. In the
eventuality that one of the parameters in
analysis falls outside the accepted range
indicated in the last row the thermodynamic
analysis is discontinued and the oligoblocker
is discarded.

| Oligo- | | | | | | | |
|---|---|---|---|---|---|---|---|
| blocker | | | Tm | | GC% | self- | Hair- |
| name | Sequence | Length | (° C.) | GC% | tail | dimers | pins |
| L861 | TCCGCACC | 23 | 76^{#} | ^{#} | ^{#} | ^{#} | ^{#} |
| iOB23 | CAGCAGTT | | | | | | |
| | TGGCCAG | | | | | | |
| | (SEQ ID | | | | | | |
| | NO: 114) | | | | | | |
| L861 | CCGCACC | 22 | 74^{#} | ^{#} | ^{#} | ^{#} | ^{#} |
| iOB22 | CAGCAGT | | | | | | |
| | TTGGCCAG | | | | | | |
| | (SEQ ID | | | | | | |
| | NO: 115) | | | | | | |
| L861 | CGCACCCA | 21 | 70^{#} | ^{#} | ^{#} | ^{#} | ^{#} |
| iOB21 | GCAGTTT | | | | | | |
| | GGCCAG | | | | | | |
| | (SEQ ID | | | | | | |
| | NO: 116) | | | | | | |
| L861 | GCACCCA | 20 | 66^{#} | ^{#} | ^{#} | ^{#} | ^{#} |
| iOB20 | GCAGTT | | | | | | |
| | TGGCCAG | | | | | | |
| | (SEQ ID | | | | | | |
| | NO: 117) | | | | | | |
| L861 | CACCCAG | 19 | 62* | 63.2* | 71.4* | -9.98^{#} | ^{#} |
| iOB19 | CAGTTT | | | | | | |
| | GGCCAG | | | | | | |
| | (SEQ ID | | | | | | |
| | NO: 118) | | | | | | |
| L861 | TCCGCAC | 23 | 76^{#} | ^{#} | ^{#} | ^{#} | ^{#} |
| iOB23/ | CCAGCAGT | | | | | | |
| 2 | TTGGCCAG | | | | | | |
| | (SEQ ID | | | | | | |
| | NO: 119) | | | | | | |
| L861 | TCCGCACC | 22 | 72^{#} | ^{#} | ^{#} | ^{#} | ^{#} |
| iOB22/ | CAGCAGT | | | | | | |
| 2 | TTGGCCA | | | | | | |
| | (SEQ ID | | | | | | |
| | NO: 120) | | | | | | |
| L861 | TCCGCAC | 21 | 68^{#} | ^{#} | ^{#} | ^{#} | ^{#} |
| iOB21/ | CCAGCAG | | | | | | |
| 2 | TTTGGCC | | | | | | |
| | (SEQ ID | | | | | | |
| | NO: 121) | | | | | | |
| L861 | TCCGCAC | 20 | 64* | 50* | 42.9* | none* | none* |
| iOB20/ | CCAGCA | | | | | | |
| 2 | GTTTGGC | | | | | | |
| | (SEQ ID | | | | | | |
| | NO: 21) | | | | | | |
| L861 | TCCGCAC | 19 | 60* | 57.9* | 42.9* | none* | none* |
| iOB19/ | CCAGCAG | | | | | | |
| 2 | TTTGG | | | | | | |
| | (SEQ ID | | | | | | |
| | NO: 20) | | | | | | |

| | | | | | | |
|---|---|---|---|---|---|---|
| Accepted values | 19- | ~60° | 40- | >25% | >−4 | >−3 |
| for the | 23 bp | C. | 60% | | Kcal/ | Kcal/ |
| parameters in | | | | | Mol | Mol |
| analysis | | | | | | |
| (Oligoblocker) | | | | | | |

IV4) Thermodynamic Analysis on All Candidate Primers and Oligoblockers

The following heterodimers combinations to consider for all the primers/oligoblocker candidates that met the thermodynamic criteria described in the previous sections:
Allele-specific primer+paired WT primer
Allele-specific primer+oligoblocker
Paired WT primer+oligoblocker.
   The results are presented in Table 25.

TABLE 25
Evaluation of heterodimers presence for all oligonucleotide candidates
evaluated for the allele-specific system.

| Oligonucleotide | | Validated? |
|---|---|---|
| combination | ΔG (Kcal/mol) | Y/N |
| L861Q i17 + L861 iP20 | −1.53 | Y |
| L861Q i18 + L861 iP20 | −1.53 | Y |
| L861Q i17 + L861 iOB19/2 | −1.81 | Y |
| L861Q i17 + L861 iOB20/2 | −1.81 | Y |
| L861Q i18 + L861 iOB19/2 | −1.81 | Y |
| L861Q i18 + L861 iOB20/2 | −1.81 | Y |
| L861 iP20 + iOB19/2 | −2.02 | Y |
| L861 iP20 + iOB20/2 | −2.02 | Y |
| Accepted values for the | >−4 Kcal/mol | Y |

parameters in analysis

The results show all oligonucleotide candidates meet the thermodynamic criteria required to ensure absence of non-specific amplification and to improve the amplification efficiency.

IV5) Design of the EGFR L861Q Wild-Type Set

To produce optimal L861Q Wild-Type primer sets, we chose to target the DNA region shown in FIG. 11.

### L861 Wild-Type Set Candidates

The tested primer candidates are disclosed in Table 26.

TABLE 26
Evaluation of all thermodynamic parameters considered to select
appropriate oligonucleotides for the L861 wild-type set.
Accepted values are shown with “*”.

| Primer | | | Tm | | GC% | self- | Hair- | Hetero- | Validated |
|---|---|---|---|---|---|---|---|---|---|
| name | Sequence | Length | (° C.) | GC% | tail | dimers | pins | dimers | (Y/N) |
| L861 | CTGTTCCCAA | 19 | 58* | 52.6* | 57.1* | −3.13* | −0.22* | −3.48* | Y |
| WT R | AGCAGCTCT | | | | | | | | |
| | (SEQ ID NO: 23) | | | | | | | | |
| L861 | GATGCTCTCC | 19 | 56* | 47.4* | 28.6* | −0.22* | −0.1* | | Y |
| WT L | AGACATTCT | | | | | | | | |
| | (SEQ ID NO: 22) | | | | | | | | |

| | | | | | | | | |
|---|---|---|---|---|---|---|---|---|
| Accepted values for the parameters | 16- | 55-60° C. | 40- | >25% | >−4 | >−3 | >−4 | Y |
| in analysis (wild-type primer set) | 22bp | ΔTm | 60% | | Kcal/ | Kcal/ | Kcal/ | |
| | | with | | | mol | mol | mol | |
| | | paired | | | | | | |
| | | primer | | | | | | |
| | | <5° C. | | | | | | |

Our results confirm the candidate primers exhibit the required characteristics. The pair was thus validated for further analysis.

### Blat Alignment of the Validated Primer Pairs in Inverse Configuration

Blat alignments (Human GRCh38/hg38) for the PCR amplicons generated by the combinations of primers meeting the thermodynamic criteria were performed.

The Blat local alignment highlighted the presence of secondary homology regions. Nevertheless, none of the secondary homology region overlapped with the primer sequences. Hence no secondary product was produced. This primer combination could hence be validated. The oligonucleotide candidates of the L861 WT set met the thermodynamic criteria required to ensure absence of non-specific amplification and to improve the amplification efficiency.

IV6) In Vitro Validation of the L861Q Primer Pairs

L861Q i18+L861 iP20+L861 iOB20/2

### Specificity

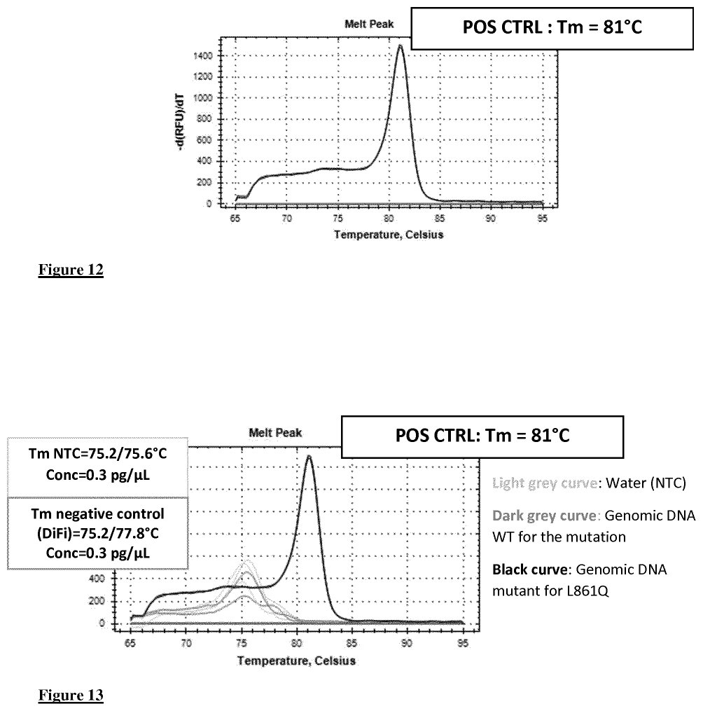

The specificity of the combination was tested. The results are presented in FIG. 12. They showed one peak-only, indicating the specificity QC criteria are met.

### Non-Specificity

Melting curves of genomic DNA WT for the L861Q mutation were compared to melting curves of commercial DNA mutant for L861Q using this oligonucleotide combination (see FIG. 13). There was not any non-specificity observed. This oligonucleotide combination was thus evaluated for efficiency.

### Efficiency

The efficiency analysis for the mutation-specific set comprised of L861Q i18+L861 iP20+L861 iOB20 was performed in conjunction with the proposed combination for the L861 WT primer set. The results are presented in Table 27.

TABLE 27
Demonstration of the efficiency of the oligonucleotide system for the detection of
EGFR L861Q.

| | | | | [DNA] | Tm specific? | | |
|---|---|---|---|---|---|---|---|
| Target | Content | Sample | Cq | ng/μl | (Y/N) | Mean | mA % |
| L861Q | Neg | DNA | 36.88 | N/A | N | | |
| i18 + L861 | control | Di-Fi | 37.67 | N/A | N | | |
| iP20 + L861 | NTC | Water | 33.38 | N/A | N | | |
| iOB20/2 | | | 36.24 | N/A | N | | |
| | Positive | Horizon | 27.63 | 0.1825 | Y | 0.175 | 36.61% |
| | control | std | 27.75 | 0.1671 | Y | | |
| L861 WT | | L861Q | 25.66 | 0.4592 | Y | 0.477 | |
| Assay | | 50% | 25.55 | 0.4957 | Y | | |
| (L861 WT | | | | | | | |
| L + L861 | | | | | | | |
| WT R) | | | | | | | |

The observed mutant allele frequency of the L861Q mutation-specific system on the L861Q WT system is 62%, close to expected 50% reported by the supplier of the positive control used for the experiment.

The efficiency is thus appropriate and this oligonucleotide combination was further evaluated for sensitivity.

### Sensitivity

The results are presented in Table 28.

TABLE 28
Demonstration of the high sensitivity of the method for the detection of the EGFR
L861Q point mutation. Serial dilutions of a commercial DNA control positive for
the L861Q mutation were performed, up to 1/10000. The system can detect the L861Q
mutation down to a frequency of 0.2 pg/μl. The sensitivity is thus very high.

| | | [DNA] | | |
|---|---|---|---|---|
| | | (ng/μl) | Tm | |
| Sample | Cq | extract | specificity | mean |
| C+ L861Q | 27.63 | 0.182500 | Y | 0.174800 |
| | 27.75 | 0.167100 | Y | |
| C+ 861 1/10 | 30.96 | 0.016750 | Y | 0.017160 |
| | 30.89 | 0.017570 | Y | |
| C+ 861 1/100 | 33.3 | 0.003115 | Y | 0.001519 |
| | 34.76 | 0.001090 | Y | |
| | 35.45 | 0.000666 | Y | |
| | 33.47 | 0.002750 | Y | |
| | 34.88 | 0.001001 | Y | |
| | 35.86 | 0.000492 | Y | |
| C+ 861 1/1000 | 36.33 | 0.000353 | N | 0.000220 |
| | 37.36 | 0.000168 | N | |
| | 36.86 | 0.000240 | N | |
| | 35.3 | 0.000739 | N | |
| | 35.27 | 0.000757 | N | |
| | 36.98 | 0.000220 | Y | |
| C+ 8611/10000 | 35.85 | 0.000499 | N | ND |
| | 37.34 | 0.000171 | N | |
| | 35.62 | 0.000588 | N | |
| | 39 | 0.000052 | N | |
| | 37.76 | 0.000126 | N | |
| | 37.75 | 0.000127 | N | |

IV7) Conclusions

Our method exhibits high specificity, efficiency and sensitivity. The results here presented confirm that among all tested primer combinations the method comprised of the mutation-specific set L861Q i18+L861 iP20+L861 iOB20/2 and of the wild-type set L861 WT L+L861 WT R is the best system to detect the EGFR L861Q.

Other systems that allow efficient detection include:
L861Q i17+L861 iP20+iOB19/2
L861Q i17+L861 iP20+iOB20/2
L861Q il8+L861 iP20+iOB19/2.

### V) EGFR S768I

EGFR S768I is a substitution mutation. The S768I mutation results in an amino acid substitution at position 768, from a Serine (S) to an Isoleucine (I). This mutation occurs within exon 20, which encodes part of the kinase domain. Nomenclature of EGFR S768I point mutation is c: 2303 G>T. It means that the 2303^{th} nucleotide on the cDNA reference sequence is a Guanine that is substituted by a Thymine.

The inventors have designed specific sets of primers allowing improved detection of the presence (or absence, or frequency) of such a mutation from a sample containing cfNA.

V1) Design for the Allele-Specific EGFR S768I Primer

Various allele-specific primers considered in the design of this invention and their thermodynamic characteristics were analyzed, as shown in Table 29.

TABLE 29
List of all the EGFR S768I allele-specific
primers considered in the design of this
invention and their thermodynamic character-
istics. Accepted values are shown with “*”.
Borderline values are shown with “#” and non-
acceptable values are shown with “§”. In the
eventuality that one of the parameters in
analysis falls outside the accepted range
indicated in the last row the thermodynamic
analysis is discontinued and the primer is
discarded.

| | | | | | | | |
|---|---|---|---|---|---|---|---|
| Primer | | | Tm | | GC% | self- | Hair- |
| name | Sequence | Length | (° C.) | GC% | tail | dimers | pins |

Conventional configuration

| | | | | | | | |
|---|---|---|---|---|---|---|---|
| S768I | AGGAAGC | 22 | 68^{§} | ^{§} | ^{§} | ^{§} | ^{§} |
| 22 | CTACGTG | | | | | | |
| | ATGGCCAT | | | | | | |
| | (SEQ ID | | | | | | |
| | NO: 122) | | | | | | |
| S768I | GGAAGCC | 21 | 66^{§} | ^{§} | ^{§} | ^{§} | ^{§} |
| 21 | TACGTGA | | | | | | |
| | TGGCCAT | | | | | | |
| | (SEQ ID | | | | | | |
| | NO: 123) | | | | | | |
| S768I | GAAGCC | 20 | 62^{§} | ^{§} | ^{§} | ^{§} | ^{§} |
| 20 | TACGTGA | | | | | | |
| | TGGCCAT | | | | | | |
| | (SEQ ID | | | | | | |
| | NO: 124) | | | | | | |
| S768I | AAGCCTAC | 19 | 58^{§} | ^{§} | ^{§} | ^{§} | ^{§} |
| 19 | GTGATGG | | | | | | |
| | CCAT | | | | | | |
| | (SEQ ID | | | | | | |
| | NO: 125) | | | | | | |
| S768I | AGCCTA | 18 | 56* | 55.6* | 57.1* | -12.9^{§} | ^{§} |
| 18 | CGTGAT | | | | | | |
| | GGCCAT | | | | | | |
| | (SEQ ID | | | | | | |
| | NO: 126) | | | | | | |
| S768I | GCCTACG | 17 | 54* | 58.8* | 57.1* | -12.9^{§} | ^{§} |
| 17 | TGATGGC | | | | | | |
| | CAT | | | | | | |
| | (SEQ ID | | | | | | |
| | NO: 127) | | | | | | |
| S768I | CCTACGTG | 16 | 50* | 56.3* | 57.1* | -12.9^{§} | ^{§} |
| 16 | ATGGCCAT | | | | | | |
| | (SEQ ID | | | | | | |
| | NO: 128) | | | | | | |

Inverse configuration

| | | | | | | | |
|---|---|---|---|---|---|---|---|
| S768I | CACACGTG | 22 | 72^{§} | ^{§} | ^{§} | ^{§} | ^{§} |
| i22 | GGGGTTG | | | | | | |
| | TCCACGA | | | | | | |
| | (SEQ ID | | | | | | |
| | NO: 129) | | | | | | |
| S768I | ACACGTG | 21 | 68^{§} | ^{§} | ^{§} | ^{§} | ^{§} |
| i21 | GGGGTT | | | | | | |
| | GTCCACGA | | | | | | |
| | (SEQ ID | | | | | | |
| | NO: 130) | | | | | | |
| S768I | CACGTGG | 20 | 66^{§} | ^{§} | ^{§} | ^{§} | ^{§} |
| i20 | GGGTTGT | | | | | | |
| | CCACGA | | | | | | |
| | (SEQ ID | | | | | | |
| | NO: 131) | | | | | | |
| S768I | ACGTGGGG | 19 | 62^{§} | ^{§} | ^{§} | ^{§} | ^{§} |
| i19 | GTTGTCCA | | | | | | |
| | CGA | | | | | | |
| | (SEQ ID | | | | | | |
| | NO: 132) | | | | | | |
| S768I | CGTGGGGGT | 18 | 60^{§} | ^{§} | ^{§} | ^{§} | ^{§} |
| i18 | TGTCCACGA | | | | | | |
| | (SEQ ID | | | | | | |
| | NO: 133) | | | | | | |
| S768I | GTGGGGGTT | 17 | 56* | 64.7* | 57.1* | -3.16* | -3. |
| i17 | GTCCACGA | | | | | | 04^{#} |
| | (SEQ ID | | | | | | |
| | NO: 35) | | | | | | |
| S768I | TGGGGGTT | 16 | 52* | 62.5* | 57.1* | -1.81* | -1. |
| i16 | GTCCACGA | | | | | | 69* |
| | (SEQ ID | | | | | | |
| | NO: 34) | | | | | | |

| | | | | | | |
|---|---|---|---|---|---|---|
| Accepted values | 16-22 | 45-56 | 40- | >25% | >−4 | >−3 |
| for the | bp | ° C. | 60% | | Kcal/ | Kcal/ |
| parameters | | | | | mol | mol |
| in analysis | | | | | | |
| (allele-specific | | | | | | |
| primer shown | | | | | | |
| in bold) | | | | | | |

V2) Paired Wild-Type Primer

Results Obtained with Primer 3

All allele-specific primers that met our criteria (see Table 29) were used on Primer3 to find a compatible paired primer. Results are summarized in Table 30.

TABLE 30
List of the only EGFR S768I paired WT- primers produced by Primer
3 and considered in the design of this invention and its
thermodynamic characteristics. Tm are calculated following
the recommended Primer3 algorithm. Accepted values are shown
with “*”. Borderline values are shown with “#”.

| | | | | | | | | | | | | Off |
|---|---|---|---|---|---|---|---|---|---|---|---|---|
| | Allele- | Paired | | | | | | | | | | target |
| Allele- | specific | WT | Paired WT | | | | | | | | | with wt |
| specific | Primer | primer | primer | | Amplicon | Tm | | GC% | self- | Hair- | Off | allele- |
| Primer | Tm | name | sequence | Length | size | (° C.) | GC% | tail | dimers | pins | target? | specific? |
| S768I | 61.44 | S768 | CATGTGCCCC | 17 | 98^{#} | 57* | 58.82* | 57.1* | -2.17* | none* | none* | none* |
| i17 | | iP17 | TCCTTCTGG | | | | | | | | | |
| | | | (SEQ ID NO: | | | | | | | | | |
| | | | 134) | | | | | | | | | |

| | | | | | | | | | |
|---|---|---|---|---|---|---|---|---|---|
| Accepted values for the parameters | 16-25 | <100bp | ΔTm | 40- | >25% | >−4 | >−3 | no | no |
| in analysis (paired WT primer) | bp | | with | 60% | | Kcal/ | Kcal/ | | |
| | | | paired | | | mol | mol | | |
| | | | primer | | | | | | |
| | | | <5° C. | | | | | | |

The paired wt primer candidates identified by Primer3 has suitable thermodynamic characteristics but produces amplicons that are considered borderline for low frequency detection of mutations from tumour-derived cfDNA fragments.

The paired-wild type primer was thus manually designed following the thermodynamic criteria described in the previous sections to try and reduce the PCR amplicon size.

### Manually Generated Primer Pair Candidates in Inverse Configuration

Paired wild-type primers were manually generated to reduce the PCR amplicon size. All primers are listed in Table 31. Their characteristics were analyzed.

TABLE 31

| | | | | | | | | | | | | Off |
|---|---|---|---|---|---|---|---|---|---|---|---|---|
| | Allele- | | | | | | | | | | | target |
| Allele- | specific | | Paired WT | | | | | | | | | with wt |
| specific | Primer | Paired WT | primer | | Amplicon | Tm | | GC % | self- | Hair- | Off | allele- |
| Primer | Tm | primer name | sequence | Length | size | (° C.) | GC % | tail | dimers | pins | target? | specific? |
| S768I | 52 | S768 iP18 | AGCCACACTG | 18 | 67* | 58^{§} | ^{§} | ^{§} | ^{§} | ^{§} | ^{§} | ^{§} |
| i16 | | | ACGTGCCT | | | | | | | | | |
| | | | (SEQ ID NO: | | | | | | | | | |
| | | | 36) | | | | | | | | | |
| | | S768 iP19 | AGCCACACTG | 19 | 67* | 62^{§} | ^{§} | ^{§} | ^{§} | ^{§} | ^{§} | ^{§} |
| | | | ACGTGCCTC | | | | | | | | | |
| | | | (SEQ ID NO: | | | | | | | | | |
| | | | 135) | | | | | | | | | |
| S768I | 56 | S768 iP18 | AGCCACACTG | 18 | 68* | 58* | 61.1* | 71.4* | −3.19* | 0.04* | no* | no* |
| i17 | | | ACGTGCCT | | | | | | | | | |
| | | | (SEQ ID NO: | | | | | | | | | |
| | | | 36) | | | | | | | | | |
| | | S768 iP19 | AGCCACACTG | 19 | 68* | 62^{§} | ^{§} | ^{§} | ^{§} | ^{§} | ^{§} | ^{§} |
| | | | ACGTGCCTC | | | | | | | | | |
| | | | (SEQ ID NO: | | | | | | | | | |
| | | | 135) | | | | | | | | | |

| | | | | | | | | | |
|---|---|---|---|---|---|---|---|---|---|
| Accepted values for the parameters | 16-25 | <100 bp | ΔTm | 40- | >25% | >−4 | >−3 | no | no |
| in analysis (paired WT primer) | bp | | with | 60% | | Kcal/ | Kcal/ | | |
| | | | paired | | | mol | mol | | |
| | | | primer | | | | | | |
| | | | <5° C. | | | | | | |

List of all potential EGFR S768I paired WT- primers in inverse configuration manually generated and considered in the design of this invention and their thermodynamic characteristics.
Tm are calculated using the Oligoanalyzer ® software.
Accepted values are shown with “*” and non-acceptable values are shown with “^{§}”.
In the eventuality that one of the parameters in analysis falls outside the accepted range indicated in the last row the thermodynamic analysis is discontinued and the primer is discarded.

The following combinations were thus found, that met the thermodynamic criteria required:
S768I i17+S768 iP18.

### Blat Alignment of the Validated Primer Pairs in Inverse Configuration

Blat alignments were produced (Human GRCh38/hg38) for the PCR amplicons generated by the combination of primers meeting the thermodynamic criteria.

The primer pair in analysis produced just one single amplicon at the expected chromosomal position. No other homologies are reported.

V3) Oligoblocker for the EGFR S768 Position

We generated several oligoblockers that target the wild-type sequence for S768 and tested their characteristics in combination with the primers.

TABLE 32

| Oligo- | | | | | | | |
|---|---|---|---|---|---|---|---|
| blocker | | | Tm | | GC % | self- | |
| name | Sequence | Length | (° C.) | GC % | tail | dimers | Hairpins |
| S768 iOB23 | CACGTGGGGGT | 23 | 78^{§} | ^{§} | ^{§} | ^{§} | ^{§} |
| | TGTCCACGCTGG | | | | | | |
| | (SEQ ID NO: 136) | | | | | | |
| S768 iOB22 | ACGTGGGGGTT | 22 | 74^{§} | ^{§} | ^{§} | ^{§} | ^{§} |
| | GTCCACGCTGG | | | | | | |
| | (SEQ ID NO: 137) | | | | | | |
| S768 iOB21 | CGTGGGGGTTG | 21 | 72^{§} | ^{§} | ^{§} | ^{§} | ^{§} |
| | TCCACGCTGG | | | | | | |
| | (SEQ ID NO: 138) | | | | | | |
| S768 iOB20 | GTGGGGGTTGTC | 20 | 68^{§} | ^{§} | ^{§} | ^{§} | ^{§} |
| | CACGCTGG (SEQ | | | | | | |
| | ID NO: 139) | | | | | | |
| S768 i0B19 | TGGGGGTTGTCC | 19 | 64* | 68.44^{#} | 71.4* | −1.81* | −1.69* |
| | ACGCTGG (SEQ | | | | | | |
| | ID NO: 196) | | | | | | |

| | | | | | | |
|---|---|---|---|---|---|---|
| Accepted values for the | 19- | ~60° C. | 40- | >25% | >−4 | >−3 |
| parameters in analysis | 23 bp | | 60% | | Kcal/ | Kcal/ |
| (Oligoblocker shown in bold) | | | | | Mol | mol |

List of all oligoblockers for the EGFR S768 codon, in conventional configuration, considered in the design of this invention and their thermodynamic characteristics.
The Tm are calculated using the Oligoanalyzer ® software.
Accepted values are shown with “*”.
Borderline values are shown with “^{#}” and non-acceptable values are shown with “^{§}”. In the eventuality that one of the parameters in analysis falls outside the accepted range indicated in the last row the thermodynamic analysis is discontinued and the oligoblocker is discarded.

The best candidate is the S768 i OB19, despite slightly high GC % and Tm, as all other combination presented excessive too high Tm. All further thermodynamic analyses were thus conducted considering S768 i OB19 as best oligoblocker candidate.

V4) Thermodynamic Analysis on All Candidate Primers and Oligoblockers

The following heterodimers combinations were considered for all the primers/oligoblocker candidates that met the thermodynamic criteria described in the previous sections:
Allele-specific primer+paired WT primer
Allele-specific primer+oligoblocker
Paired WT primer+oligoblocker.

The results are presented in Table 33.

TABLE 33
Evaluation of heterodimers presence for all oligonucleotide
candidates evaluated for the allele-specific system.

| Oligonucleotide | ΔG | Validated? |
|---|---|---|
| combination | (Kcal/mol) | Y/N |
| S768I i17 + S768 iP18 | −3.7 | Y |
| S768I i17 + S768 iOB19 | −3.16 | Y |
| S768 iOB19 + S768 iP18 | −3.7 | Y |
| Accepted values for the | >−4 Kcal/mol | Y |
| parameters in analysis | | |

The results show all oligonucleotide candidates meet the thermodynamic criteria required to ensure absence of non-specific amplification and to improve the amplification efficiency.

V5) Design of the S768 Wild-Type Set

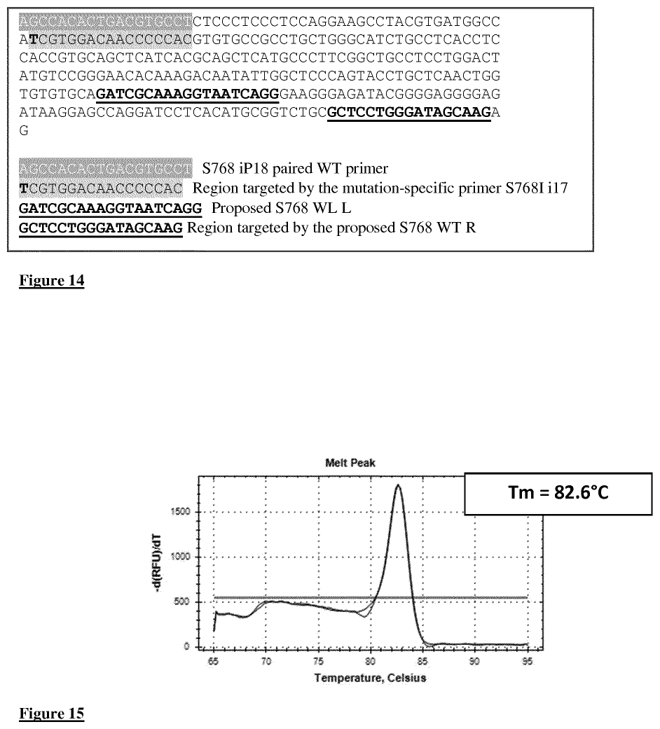

To produce optimal S768 wild-type sets, we chose to target the DNA region shown in FIG. 14.

The tested primer candidates are disclosed in Table 34.

TABLE 34

| | | | | | GC % | self- | Hair- | Hetero- | Validated |
|---|---|---|---|---|---|---|---|---|---|
| Primer name | Sequence | Length | Tm (° C.) | GC % | tail | dimers | pins | dimers | (Y/N) |
| S768 WT L | GATCGCAAAGGTAATCAG | 19 | 56 | 47.4 | 42.9 | −1.41 | 0.28 | −3.83^{#} | Y |
| | G (SEQ ID NO: 38) | | | | | | | | |
| S768 WT R | TCTTGCTATCCCAGGAGC | 18 | 56 | 55.6 | 71.4 | −1.53 | −1.41 | | Y |
| | (SEQ ID NO: 39) | | | | | | | | |
| Accepted values for the | | 16- | 55-60° C. | 40- | >25% | >−4 | >−3 | >−4 | Y |
| parameters in analysis | | 22 bp | ΔTm | 60% | | Kcal/ | Kcal/ | Kcal/ | |
| (wild-type primer set) | | | with | | | mol | mol | mol | |
| | | | paired | | | | | | |
| | | | primer | | | | | | |
| | | | <5° C. | | | | | | |

Evaluation of all S768 thermodynamic parameters considered to select appropriate oligonucleotides for the IntPlex ® wild-type set.
Accepted values are shown with “^{#}”.

Our results confirm the candidate primers exhibit the required characteristics. The pair was thus validated for further analysis.

### Blat Alignment of the Validated Primer Pairs in Inverse Configuration

Blat alignments were produced (Human GRCh38/hg38) for the PCR amplicons generated by the combination of primers meeting the thermodynamic criteria.

The Blat local alignment highlighted the presence of some secondary homology region. Nevertheless, by analyzing the structure and extension of the homology region reported on chrX we concluded that it would at most produce a partial annealing region for the S768 WT L primer but, as no homology is present in the target region of the S768 WT R primer, no secondary product would be produced. None of the other homology regions spans the primer target sequence. This primer combination could hence be validated.

The oligonucleotide candidates of the S768 WT set met the thermodynamic criteria required to ensure absence of non-specific amplification and to improve the amplification efficiency.

V6) In Vitro Validation of the S768I Primer Pairs

S768I i16+S768 iP18+S768 iOB19

### Specificity

The specificity of the combination was tested. The results showed one peak-only, indicating the specificity QC criteria are met.

### Non-Specificity

A slight amplification was observed in the negative control and in the NTC, with a 4° C. Tm difference from the expected amplification peak of the positive control. Moreover, the concentration of the non-specific amplification is <0.5 pg/μl, in compliance with IntPlex® acceptance QC criteria.

As the Tm difference between the tested mutation-specific primer S768I i16 and the paired WT primer S768 iP18 is 6° C., the mutation-specific primer S768I i17 will be also tested.

S768I i17+S768 iP18+S768 iOB19

### Specificity

The specificity of the combination was tested. The results are presented in FIG. 15. They showed one peak-only, indicating that specificity QC criteria are met.

### Non-Specificity

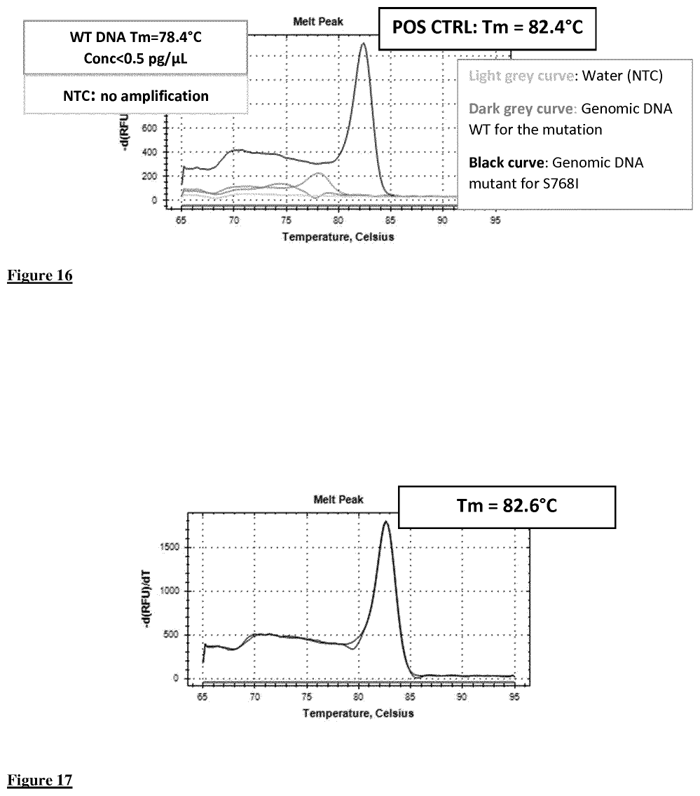

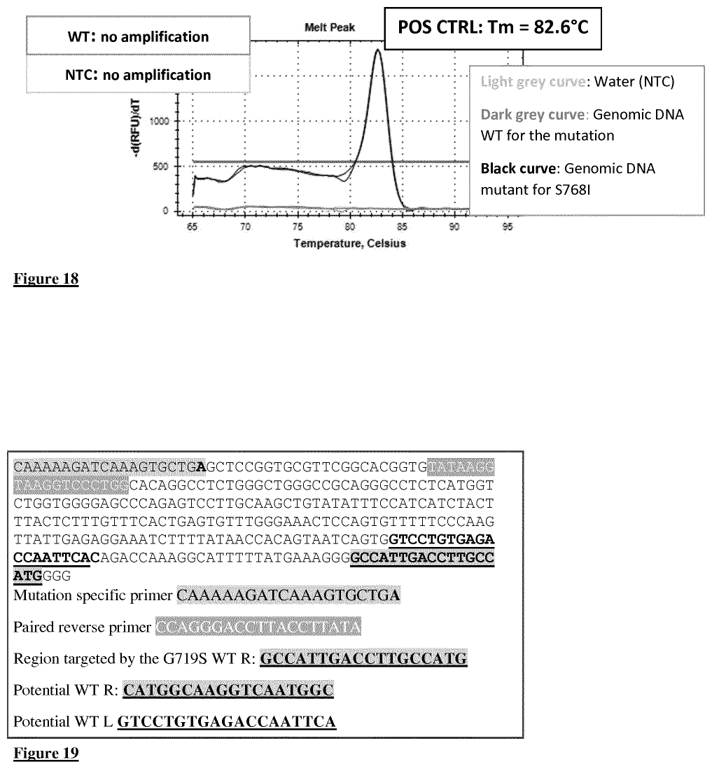

Melting curves of genomic DNA WT for the S768I mutation were compared to melting curves of commercial DNA mutant for S768I using this oligonucleotide combination (see FIG. 16). There was not any non-specificity observed, with no amplification observed in the NTC and in the WT DNA control. This oligonucleotide combination was thus evaluated for efficiency.

### Efficiency

The efficiency analysis for the mutation-specific set comprised of S768 i17+S768 iP18+S768 iOB19 was performed in conjunction with the proposed combination for the S768 WT primer set. The results are presented in Table 35.

TABLE 35
Demonstration of the efficiency of the oligonucleotide system for the detection
of EGFR S768I.

| | | | | [DNA] | Tm specific? | | |
|---|---|---|---|---|---|---|---|
| Target | Content | Sample | Cq | ng/μl | (Y/N) | Mean | mA % |
| S768 i17 + | Neg | Di-Fi | N/A | N/A | | | |
| S768 iP18 + | control | DNA | 39.04 | N/A | N | | |
| S768 iO B19 | NTC | Water | N/A | N/A | | | |
| | | | N/A | N/A | | | |
| | Positive | Horizon | 28.2 | 0.08 | Y | 0.085 | 28.33% |
| | control | std L861Q | 28.15 | 0.09 | Y | 0.300 | |
| S768 WT L + | | 50% | 26.38 | 0.29 | Y | | |
| L768 WT R) | | | 36.31 | 0.31 | Y | | |

The observed mutant allele frequency of the S768I mutation-specific system on the S768 WT system is 28.33%, close to expected 50% reported by the supplier of the positive control used for the experiment.

The efficiency is thus appropriate and this oligonucleotide combination was further evaluated for sensitivity.

### Sensitivity

The results are presented in Table 36.

TABLE 36
Demonstration of the high sensitivity of the method in analysis for the
detection of the EGFR S768I point mutation. Serial dilutions of a
commercial DNA control positive for the S768I mutation at expected
mutational load of 0.08 ng/μl were performed, up to 1/1000.

| | | [DNA] | | | |
|---|---|---|---|---|---|
| | | (ng/μl) | Tm | | |
| | Cq | extract | specificity | mean | theoretical |
| C+ S768I | 28.2 | 0.08202 | Y | 0.0836 | 0.08 |
| | 28.15 | 0.08514 | Y | | |
| C+ S768I1/10 | 31.75 | 0.00692 | Y | 0.0086 | 0.008 |
| | 31.19 | 0.01028 | Y | | |
| C+ S768I1/100 | 34.8 | 0.00083 | Y | 0.0011 | 0.0008 |
| | 34.45 | 0.00106 | Y | | |
| | 33.56 | 0.00197 | Y | | |
| | 34.4 | 0.00110 | Y | | |
| | 34.24 | 0.00123 | Y | | |
| | 35.91 | 0.00038 | Y | | |
| C+ S768I1/1000 | 37.65 | 0.00011 | N | 0.0002 | 0.00008 |
| | 39.12 | 0.00004 | N | | |
| | 35.61 | 0.00047 | N | | |
| | 36.86 | 0.00020 | Y | | |

The system can detect the S768I mutation down to a frequency of 0.04 pg/μl. The sensitivity is thus very high.

V7) Conclusions

Our method allows detection of the S768I mutation with high specificity, efficiency and sensitivity.

Among the tested primers, the optimal combination is comprised of the mutation-specific set S768I i17+S768 iP18+S768 iOB19 and of the wild-type set S768 WT L+S768 WT R.

Other suitable primer combinations include:
S768I i16+S768 iP18+S768 iOB19.

### VI) G719S

The G719S mutation results in an amino acid substitution at position 719, from a glycine (G) to a serine (S). This mutation occurs within exon 18, which encodes part of the kinase domain. Nomenclature of EGFR G719S point mutation is c: 2155 G>A. It means that the 2155^{th} nucleotide on the cDNA reference sequence is a Guanine that is substituted by an Adenine.

The inventors have designed specific sets of primers allowing improved detection of the presence (or absence, or frequency) of such a mutation from a sample containing cfNA.

VI1) Design of the Allele-Specific EGFR G719S Primer

Various allele-specific primers were considered in the design of this invention and their thermodynamic characteristics were analyzed, as shown in Table 37.

TABLE 37

| | | | | | | | |
|---|---|---|---|---|---|---|---|
| Primer | | | Tm | | GC % | self- | |
| name | Sequence | Length | (° C.) | GC % | tail | dimers | Hairpins |

Conventional configuration

| | | | | | | | |
|---|---|---|---|---|---|---|---|
| G7195 22 | ATCAAAAAGAT | 22 | 58^{§} | ^{§} | ^{§} | ^{§} | ^{§} |
| | CAAAGTGCTGA | | | | | | |
| | (SEQ ID NO: 140) | | | | | | |
| G7195 21 | TCAAAAAGATCA | 21 | 56^{§} | ^{§} | ^{§} | ^{§} | ^{§} |
| | AAGTGCTGA | | | | | | |
| | (SEQ ID NO: 141) | | | | | | |
| G7195 20 | CAAAAAGATCAA | 20 | 54* | 35* | 57.1* | −1.41* | −0.2* |
| | AGTGCTGA (SEQ | | | | | | |
| | ID NO: 25) | | | | | | |
| G7195 19 | AAAAAGATCAAA | 19 | 50* | 31.6* | 57.1* | −1.41* | −0.2* |
| | GTGCTGA (SEQ | | | | | | |
| | ID NO: 24) | | | | | | |
| G7195 18 | AAAAGATCAAAG | 18 | 48* | 33.3* | 57.1* | −1.41* | −0.2* |
| | TGCTGA (SEQ ID | | | | | | |
| | NO: 142) | | | | | | |
| G7195 17 | AAAGATCAAAGT | 17 | 46* | 35.3* | 57.1* | −1.41* | −0.2* |
| | GCTGA (SEQ ID | | | | | | |
| | NO: 143) | | | | | | |
| G7195 16 | AAGATCAAAGT | 16 | 44* | 37.5* | 57.1* | −1.41* | −0.2* |
| | GCTGA (SEQ ID | | | | | | |
| | NO: 144) | | | | | | |

Inverse configuration

| | | | | | | | |
|---|---|---|---|---|---|---|---|
| G7195 i22 | CCGTGCCGAACG | 22 | 73^{§} | ^{§} | ^{§} | ^{§} | ^{§} |
| | CACCGGAGCT | | | | | | |
| | (SEQ ID NO: 145) | | | | | | |
| G719S i21 | CGTGCCGAACGC | 21 | 72^{§} | ^{§} | ^{§} | ^{§} | ^{§} |
| | ACCGGAGCT | | | | | | |
| | (SEQ ID NO: 146) | | | | | | |
| G719S i20 | GTGCCGAACGCA | 20 | 68^{§} | ^{§} | ^{§} | ^{§} | ^{§} |
| | CCGGAGCT (SEQ | | | | | | |
| | ID NO: 147) | | | | | | |
| G719S i19 | TGCCGAACGCAC | 19 | 64^{§} | ^{§} | ^{§} | ^{§} | ^{§} |
| | CGGAGCT (SEQ | | | | | | |
| | ID NO: 148) | | | | | | |
| G719S i18 | GCCGAACGCACC | 18 | 62^{§} | ^{§} | ^{§} | ^{§} | ^{§} |
| | GGAGCT (SEQ ID | | | | | | |
| | NO: 149) | | | | | | |
| G719S i17 | CCGAACGCACCG | 17 | 58^{§} | ^{§} | ^{§} | ^{§} | ^{§} |
| | GAGCT (SEQ ID | | | | | | |
| | NO: 150) | | | | | | |
| G719S i16 | GAACGCACCGGA | 16 | 54* | 68.8^{§} | 71.4^{§} | ^{§} | ^{§} |
| | AGCT (SEQ ID | | | | | | |
| | NO: 151) | | | | | | |

| | | | | | | |
|---|---|---|---|---|---|---|
| Accepted values for the | 16-22 | 45- | 40- | >25% | >−4 | >−3 |
| parameters in analysis | bp | 55° C. | 60% | | Kcal/ | Kcal/ |
| (allele-specific primer | | | | | | |
| shown in bold) | | | | | mol | mol |

List of all the EGFR G7195 allele-specific primers considered in the design of this invention and their thermodynamic characteristics.
Accepted values are shown with “*”.
Borderline values are shown with “#” and non-acceptable values are shown with “^{§}”.
In the eventuality that one of the parameters in analysis falls outside the accepted range indicated in the last row the thermodynamic analysis is discontinued and the primer is discarded.

This preliminary analysis suggested that primer design in inverse configuration is thermodynamically less stable.

VI2) Paired Wild-Type Primer

Results Obtained with Primer 3

All allele-specific primers that met our criteria (see Table 37) were used on Primer3 to find a compatible paired primer. Results are summarized in Table 38.

TABLE 38

| | Allele- | | | | | | | | | | | |
|---|---|---|---|---|---|---|---|---|---|---|---|---|
| | specific | | | | | | | | | | | Off |
| | Primer | | | | | | | | | | | target |
| Allele- | Tm | | Paired | | | | | | | | | with wt |
| specific | (Primer | Paired WT | WT primer | | Amplicon | Tm | | GC % | self- | Hair- | Off | allele- |
| Primer | 3) | primer name | sequence | Length | size | (° C.) | GC % | tail | dimers | pins | target? | specific? |
| G719S20 | 55 | G719S P19 | CTGTGCCAGG | 19 | 66* | 59.7^{#} | 63.2* | 57.1* | -1.46* | none* | no* | no* |
| | | P3 | GACCTTACC | | | | | | | | | |
| | | | (SEQ ID NO: | | | | | | | | | |
| | | | 152) | | | | | | | | | |

| | | | | | | | | | |
|---|---|---|---|---|---|---|---|---|---|
| Accepted values for the parameters in | 16-25 | <100 bp | ΔTm | 40- | >25% | >−4 | >−3 | no | no |
| analysis (paired WT primer) | bp | | with | 60% | | Kcal/ | Kcal/ | | |
| | | | paired | | | mol | mol | | |
| | | | primer | | | | | | |
| | | | <5° C. | | | | | | |

List of the only EGFR G719S paired WT- primers produced by Primer 3 and considered in the design of this invention and their thermodynamic characteristics.
Tm are calculated following the recommended Primer3 algorithm.
Accepted values are shown with “*”.
Borderline values are shown with “^{#}”.
In the eventuality that one of the parameters in analysis falls outside the accepted range indicated in the last row the thermodynamic analysis is discontinued and the primer is discarded.

The only suitable paired primer obtained using the Primer3 software has a very borderline Tm difference with all mutation-specific primer candidates. A manual design of the paired WT primer was thus favored to reduce the Tm difference and ensure optimal amplification efficiency.

### Manually Generated Primer Pair Candidates

In view of the unsuccessful generation of a paired primer using conventional tools, we designed such primers manually. All designed primers are listed in Table 39. Their characteristics were analyzed.

TABLE 39

| | | | | | | | | | | | | Off |
|---|---|---|---|---|---|---|---|---|---|---|---|---|
| | Allele- | | | | | | | | | | | target |
| Allele- | specific | | Paired WT | | | | | | | | | with wt |
| specific | Primer | Paired WT | primer | | Amplicon | Tm | | GC % | self- | Hair- | Off | allele- |
| Primer | Tm | primer name | sequence | Length | size | (° C.) | GC % | tail | dimers | pins | target? | specific? |
| G719S | 54 | G719 P19 | CCAGGGACCT | 19 | 61* | 56* | 47.4* | 28.6* | −1.46* | −1.34* | no* | no* |
| 20 | | | TACCTTATA | | | | | | | | | |
| | | | (SEQ ID NO: | | | | | | | | | |
| | | | 26) | | | | | | | | | |

| | | | | | | | | | |
|---|---|---|---|---|---|---|---|---|---|
| Accepted values for the parameters | 16-25 | <100 bp | ΔTm | 40- | >25% | >−4 | <−3 | no | no |
| in analysis (paired WT primer) | bp | | with | 60% | | Kcal/ | Kcal/ | | |
| | | | paired | | | mol | mol | | |
| | | | primer | | | | | | |
| | | | <5° C. | | | | | | |

List of all potential EGFR G719S paired WT- primers in conventional configuration manually generated and considered in the design of this invention and their thermodynamic characteristics.
Tm are calculated using the Oligoanalyzer ® software.
Accepted values are shown with “*”.

The following combinations were found, that met the thermodynamic criteria required:
G719S 20+G719XP19
G719S 19+G719XP19.

### Blat Alignment of the Validated Primer Pairs in Conventional Sense

Blat alignments (Human GRCh38/hg38) for the PCR amplicon generated by the combinations of primers meeting the thermodynamic criteria were performed.

All primer pairs produced just one single amplicon at the expected chromosomal position. No other homologies are reported.

VI3) Oligoblocker Design for the EGFR G719 Position

We generated several oligoblockers that target the wild-type sequence for G719 and tested their characteristics in combination with the primers.

TABLE 40

| Oligo- | | | | | | | |
|---|---|---|---|---|---|---|---|
| blocker | | | Tm | | GC % | self- | |
| name | Sequence | Length | (° C.) | GC % | tail | dimers | Hairpins |
| G 719 OB23 | CAAAGTGCTGG | 23 | 76^{§} | ^{§} | ^{§} | ^{§} | ^{§} |
| | GCTCCGGTGCGT | | | | | | |
| | (SEQ ID NO: 153) | | | | | | |
| G 719 OB22 | CAAAGTGCTGG | 22 | 74^{§} | ^{§} | ^{§} | ^{§} | ^{§} |
| | GCTCCGGTGCG | | | | | | |
| | (SEQ ID NO: 154) | | | | | | |
| G 719 OB21 | CAAAGTGCTGG | 21 | 70^{§} | ^{§} | ^{§} | ^{§} | ^{§} |
| | GCTCCGGTGC | | | | | | |
| | (SEQ ID NO: 155) | | | | | | |
| G 719 OB20 | CAAAGTGCTGG | 20 | 66^{§} | ^{§} | ^{§} | ^{§} | ^{§} |
| | GCTCCGGTG | | | | | | |
| | (SEQ ID NO: 156) | | | | | | |
| G 719 OB19 | CAAAGTGCTGG | 19 | 62* | 63.2^{#} | 71.4* | −6.54^{§} | § |
| | GCTCCGGT (SEQ | | | | | | |
| | ID NO: 157) | | | | | | |
| G 719 | AAAAGATCAAA | 23 | 68^{§} | ^{§} | ^{§} | ^{§} | ^{§} |
| OB23/2 | GTGCTGGGCTCC | | | | | | |
| | (SEQ ID NO: 158) | | | | | | |
| G 719 | AAAGATCAAAG | 22 | 66^{§} | ^{§} | ^{§} | ^{§} | ^{§} |
| OB22/2 | TGCTGGGCTCC | | | | | | |
| | (SEQ ID NO: 159) | | | | | | |
| G 719 | AAGATCAAAGT | 21 | 64^{§} | ^{§} | ^{§} | ^{§} | ^{§} |
| OB21/2 | GCTGGGCTCC | | | | | | |
| | (SEQ ID NO: 160) | | | | | | |
| G 719 | AGATCAAAGTG | 20 | 62^{§} | ^{§} | ^{§} | ^{§} | ^{§} |
| OB20/2 | CTGGGCTCC | | | | | | |
| | (SEQ ID NO: 161) | | | | | | |
| G 719 | GATCAAAGTGC | 19 | 60* | 57.9* | 85.7* | −1.41* | none* |
| OB19/2 | TGGGCTCC (SEQ | | | | | | |
| | ID NO: 28) | | | | | | |
| G 719 | ATCAAAGTGCT | 18 | 56* | 55.6* | 85.7* | none* | none* |
| OB18/2 | GGGCTCC (SEQ | | | | | | |
| | ID NO: 27) | | | | | | |

| | | | | | | |
|---|---|---|---|---|---|---|
| Accepted values for the | 19- | ~60° C. | 40- | >25% | >−4 | >−3 |
| parameters in analysis | 23 bp | | 60% | | Kcal/ | Kcal/ |
| (Oligoblocker shown in bold) | | | | | Mol | mol |

List of all potential oligoblockers for the G719 codon, in conventional configuration, considered in the design of this invention and their thermodynamic characteristics.
Tm are calculated using the Oligoanalyzer ® software.
Accepted values are shown with “*”.
Borderline values are shown with “#” and non-acceptable values are shown with “§”.
In the eventuality that one of the parameters in analysis falls outside the accepted range indicated in the last row the thermodynamic analysis is discontinued and the oligoblocker is discarded.

When the variant position is kept at the middle of the potential G719 oligoblocker, the self-dimerization values are too high. By shifting the variant position towards the 3′ end of the oligoblocker more favorable thermodynamic values were obtained.

The best oligoblockers were G719 BO19/2 and G719 BO18/2 that were thus considered for all further thermodynamic analyses.

VI4) Thermodynamic Analysis on All Candidate Primers and Oligoblockers

The following heterodimers combinations were considered for all the primers/oligoblocker candidates that met the thermodynamic criteria described in the previous sections:
Allele-specific primer+paired WT primer
Allele-specific primer+oligoblocker
Paired WT primer+oligoblocker.

TABLE 41
Evaluation of heterodimers presence for all oligonucleotide candidates
evaluated for the allele-specific system.

| | ΔG | Validated? |
|---|---|---|
| Oligonucleotide combination | (Kcal/mol) | Y/N |
| G 719 OB19/2 + G719X P19 | −3.41 | Y |
| G 719 OB19/2 + G719S 20 | −1.41 | Y |
| G 719 OB19/2 + G719S 19 | −1.41 | Y |
| G 719 OB18/2 + G719X P19 | −3.41 | Y |
| G 719 OB18/2 + G719S 20 | −0.31 | Y |
| G719 OB18/2 + G719S 19 | −0.31 | Y |
| G719S 19 + G719X P19 | −0.34 | Y |
| G719S 20 + G719X P19 | −0.34 | Y |
| Accepted values for the | >−4 | Y |
| parameters in analysis | Kcal/mol | |

The results show all oligonucleotide candidates meet the thermodynamic criteria required to ensure absence of non-specific amplification and to improve the amplification efficiency.

VI5) Design of the EGFR G719S Wild-Type Set

To produce optimal G719S Wild-Type primer sets we chose to target the DNA region shown in FIG. 19.

The tested primer candidates are disclosed in Table 42.

TABLE 42

| | | | | | GC % | self- | Hair- | Hetero- | Validated |
|---|---|---|---|---|---|---|---|---|---|
| Primer name | Sequence | Length | Tm (° C.) | GC % | tail | dimers | pins | dimers | (Y/N) |
| G719 WT L | GTCCTGTGAGACCAATTCA | 19 | 56* | 47.4* | 28.6* | −2.16* | −0.2* | −2.78* | Y |
| | (SEQ ID NO: 29) | | | | | | | | |
| G719 WT R | CATGGCAAGGTCAATGGC | 18 | 56* | 55.6* | 57.1* | −2.17* | −0.1* | | Y |
| | (SEQ ID NO: 30) | | | | | | | | |

| | | | | | | | | |
|---|---|---|---|---|---|---|---|---|
| Accepted values for the | 16- | 55-60° C. | 40- | >25% | >−4 | >−3 | >−4 | Y |
| parameters in analysis | 22 bp | ΔTm | 60% | | Kcal/ | Kcal/ | Kcal/ | |
| (wild-type primer set) | | with | | | mol | mol | mol | |
| | | paired | | | | | | |
| | | primer | | | | | | |
| | | <5° C. | | | | | | |

Evaluation of all thermodynamic parameters considered to select appropriate oligonucleotides for the IntPlex ® G719 wild-type set.
Accepted values are shown with “*”.

Our results confirm that the candidate primers exhibit the required characteristics. The pair was thus validated for further analysis.

### Blat Alignment of the Validated Primer Pairs in Conventional Sense

Blat alignments (Human GRCh38/hg38) were performed for the PCR amplicons generated by the combinations of primers meeting the thermodynamic criteria.

The oligonucleotide candidates of the G719 WT set produced one single amplicon, thus meeting the thermodynamic criteria required to ensure absence of non-specific amplification and to improve the amplification efficiency.

VI6) In Vitro Validation of the EGFR G719S Primer Pairs

### G719S 20+G719 P19+G719 OB19

### Specificity

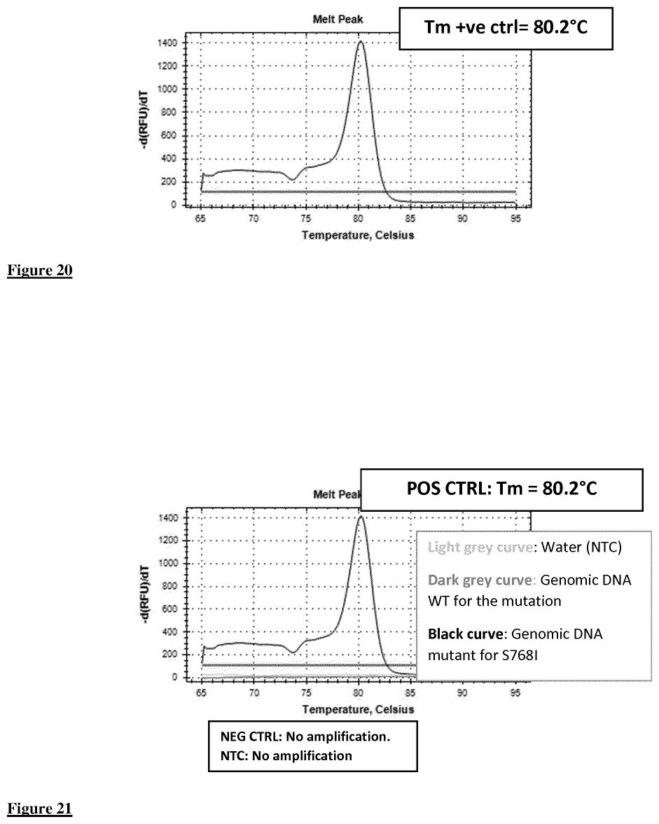

The specificity of the combination was tested. The results are presented in FIG. 20. They showed one peak-only, indicating the specificity QC criteria are met.

### Non-Specificity

Melting curves of genomic DNA WT for the G719S mutation were compared to melting curves of commercial DNA mutant for G719S using this oligonucleotide combination (see FIG. 21). There is not any non-specificity observed. This oligonucleotide combination was thus evaluated for efficiency.

### Efficiency

The efficiency analysis for the mutation-specific set comprised of G719S 20+G719 P19+G719 OB19 was performed in conjunction with the proposed combination for the G719 WT primer set. The results are presented in Table 43.

TABLE 43
Demonstration of the efficiency of the oligonucleotide system for the detection of
EGFR S791S.

| | | | | [DNA] | Tm specific? | | |
|---|---|---|---|---|---|---|---|
| Target | Content | Sample | Cq | ng/μl | (Y/N) | Mean | mA % |
| G719S 20 + | Neg | Di-Fi | N/A | N/A | N | | |
| G719 P19 + | control | DNA | N/A | N/A | N | | |
| G719 OB19 | NTC | Water | N/A | N/A | N | | |
| | | | N/A | N/A | N | | |
| | Positive | Horizon | 27.51 | 0.13305 | Y | 0.126 | 44.97% |
| | control | std | 27.68 | 0.11819 | Y | | |
| G719 WT | | L861Q | 26.46 | 0.27645 | Y | 0.279 | |
| Assay | | 50% | 26.43 | 0.28229 | Y | | |

The target G719 WT corresponds to the combination G719 WL L+G719 WT R.

The observed mutant allele frequency of the G719S mutation-specific system on the G719 WT system is 44.97%, close to expected 50% reported by the supplier of the positive control used for the experiment. The efficiency was thus appropriate and this oligonucleotide combination was further evaluated for sensitivity.

Sensitivity

The results are presented in Table 44.

TABLE 44
Demonstration of the high sensitivity of the method for the detection
of the EGFR G719S point mutation. Serial dilutions of a
commercial DNA control positive for the S719S mutation
were performed, up to 1/10000.

| | | | [DNA] | | |
|---|---|---|---|---|---|
| | | | (ng/μl) | Tm | |
| Target | Sample | Cq | extract | specificity | mean |
| B2/G719S 20 | C+ | 27.51000 | 0.13305 | Y | 0.15960 |
| | | 27.68000 | 0.11819 | Y | |
| | C+ 1/10 | 30.38000 | 0.01803 | Y | 0.01618 |
| | | 30.71000 | 0.01432 | Y | |
| | C+ 1/100 | 34.76000 | 0.00085 | Y | 0.00057 |
| | | 36.11000 | 0.00033 | Y | |
| | | N/A | | | |
| | | 35.92000 | 0.00038 | Y | |
| | | N/A | | | |
| | | 34.98000 | 0.00073 | Y | |
| | C+ 1/1000 | N/A | | | 0.00017 |
| | | N/A | | | |
| | | N/A | | | |
| | | N/A | | | |
| | | 37.06000 | 0.00017 | Y | |
| | | N/A | | | |
| | C+ 1/10000 | N/A | | | ND |
| | | N/A | | | |
| | | N/A | | | |
| | | N/A | | | |
| | | N/A | | | |
| | | N/A | | | |

The system can detect the G719S mutation down to a frequency of 0.1 pg/μl. The sensitivity is thus very high.

VI7) Conclusions

Our method has high specificity, efficiency and sensitivity.

The results here presented confirm that the best system to detect the EGFR G719S mutation is comprised of the mutation-specific set G719S 20+G719 P19+G719 OB19 and of the wild-type set G719 WT L+G719 WT R.

Other possible oligonucleotide systems include:
G719S 19+G719 P19+G719 OB18
G719S 19+G719 P19+G719 OB19
G719S 20+G719 P19+G719 OB18.

### VII) G719A

EGFR G719A is a substitution mutation. The G719A mutation results in an amino acid substitution at position 719, from a glycine (G) to an alanine (A). This mutation occurs within exon 18 which encodes part of the kinase domain. Nomenclature of the EGFR G719A point mutation is c: 2156 G>C. This means that the 2156^{th} nucleotide on the cDNA reference sequence is a Guanine that is substituted by a Cytosine.The inventors have designed specific sets of primers allowing improved detection of the presence (or absence, or frequency) of such a mutation from a sample containing cfNA.

VII1) Design of the Allele-Specific EGFR G719A Primer

Various allele-specific primers were considered in the design of this invention and their thermodynamic characteristics were analyzed, as shown in Table 45.

TABLE 45

| | | | | | | | |
|---|---|---|---|---|---|---|---|
| Primer | | | Tm | | GC % | self- | |
| name | Sequence | Length | (° C.) | GC % | tail | dimers | Hairpins |

Conventional configuration

| | | | | | | | |
|---|---|---|---|---|---|---|---|
| G719A 22 | TCAAAAAGATC | 22 | 62^{§} | ^{§} | ^{§} | ^{§} | ^{§} |
| | AAAGTGCTGGC | | | | | | |
| | (SEQ ID NO: 162) | | | | | | |
| G719A 21 | CAAAAAGATCA | 21 | 6^{§#} | ^{§} | ^{§} | ^{§} | ^{§} |
| | AAGTGCTGGC | | | | | | |
| | (SEQ ID NO: 163) | | | | | | |
| G719A 20 | AAAAAGATCAAA | 20 | 56* | 40* | 71.4* | −1.41* | none* |
| | GTGCTGGC (SEQ | | | | | | |
| | ID NO: 33) | | | | | | |
| G719A 19 | AAAAGATCAAAG | 19 | 54* | 42.1* | 71.4* | −1.41* | none* |
| | TGCTGGC (SEQ | | | | | | |
| | ID NO: 32) | | | | | | |
| G719A 18 | AAAGATCAAAGT | 18 | 52* | 44.4* | 71.4* | −1.41* | none* |
| | GCTGGC (SEQ ID | | | | | | |
| | NO: 31) | | | | | | |
| G719A 17 | AAGATCAAAGTG | 17 | 50* | 47.1* | 71.4* | −1.41* | none* |
| | CTGGC (SEQ ID | | | | | | |
| | NO: 164) | | | | | | |
| G719A16 | AGATCAAAGTGC | 16 | 48* | 50* | 71.4* | −1.41* | none* |
| | TGGC (SEQ ID | | | | | | |
| | NO: 165) | | | | | | |

Inverse configuration

| | | | | | | | |
|---|---|---|---|---|---|---|---|
| G719A i22 | ACCGTGCCGAA | 22 | 76^{§} | ^{§} | ^{§} | ^{§} | ^{§} |
| | CGCACCGGAGG | | | | | | |
| | (SEQ ID NO: 166) | | | | | | |
| G719A i21 | CCGTGCCGAACG | 21 | 74^{§} | ^{§} | ^{§} | ^{§} | ^{§} |
| | CACCGGAGG | | | | | | |
| | (SEQ ID NO: 167) | | | | | | |
| G719A i20 | CGTGCCGAACGC | 20 | 70^{§} | ^{§} | ^{§} | ^{§} | ^{§} |
| | ACCGGAGG (SEQ | | | | | | |
| | ID NO: 168) | | | | | | |
| G719A i19 | GTGCCGAACGCA | 19 | 66^{§} | ^{§} | ^{§} | ^{§} | ^{§} |
| | CCGGAGG (SEQ | | | | | | |
| | ID NO: 169) | | | | | | |
| G719A i18 | TGCCGAACGCAC | 18 | 62^{§} | ^{§} | ^{§} | ^{§} | ^{§} |
| | CGGAGG (SEQ ID | | | | | | |
| | NO: 170) | | | | | | |
| G719A i17 | GCCGAACGCACC | 17 | 60^{§} | ^{§} | ^{§} | ^{§} | ^{§} |
| | GGAGG (SEQ ID | | | | | | |
| | NO: 171) | | | | | | |
| G719A i16 | CCGAACGCACCG | 16 | 56* | 75^{§} | 85.7^{§} | ^{§} | ^{§} |
| | GAGG (SEQ ID | | | | | | |
| | NO: 172) | | | | | | |

| | | | | | | |
|---|---|---|---|---|---|---|
| Accepted values for the | 16-22 | 45- | 40- | >25% | >−4 | >−3 |
| parameters in analysis (allele- | bp | 55° C. | 60% | | Kcal/ | Kcal/ |
| specific primer shown in bold) | | | | | mol | mol |

List of all the EGFR G719A allele-specific primers considered in the design of this invention and their thermodynamic characteristics.
Accepted values are shown with “*”.
Non-acceptable values are shown with “^{§}”.
In the eventuality that one of the parameters in analysis falls outside the accepted range indicated in the last row the thermodynamic analysis is discontinued and the primer is discarded.

This preliminary analysis suggested that primer design in inverse configuration is thermodynamically less favorable.

VII2) Paired Wild-Type Primer

As the EGFR G719A assay is designed to target the same codon of the validated EGFR G719S assay (see chapter VI), it was extremely likely that the validated paired wild-type primer G719 P19 would work fine in combination with the proposed G719A mutation-specific primer candidates proposed in section VIII 1. A thermodynamic analysis was performed to exclude the presence of hetero-dimers with the mutation-specific primer candidates.

TABLE 46

| | | | | | | | | | | | | Off |
|---|---|---|---|---|---|---|---|---|---|---|---|---|
| | Allele- | | | | | | | | | | | target |
| Allele- | specific | | Paired WT | | | | | | | | | with wt |
| specific | Primer | Paired WT | primer | | Amplicon | Tm | | GC % | self- | Hair- | Off | allele- |
| Primer | Tm | primer name | sequence | Length | size | (° C.) | GC % | tail | dimers | pins | target? | specific? |
| G719A | 56 | G719 P19 | CCAGGGACCT | 19 | 61* | 56* | 47.4* | 28.6* | −1.46* | −1.34* | no* | no* |
| 20 | | | TACCTTATA | | | | | | | | | |
| G719A | 54 | | (SEQ ID NO: | | 60* | | 47.4* | 28.6* | −1.46* | −1.34* | no* | no* |
| 19 | | | 26) | | | | | | | | | |
| G719A | 52 | | | | 59* | | 47.4* | 28.6* | −1.46* | −1.34* | no* | no* |
| 18 | | | | | | | | | | | | |
| G719A | 50# | | | | 58* | | 47.4* | 28.6* | −1.46* | −1.34* | no* | no* |
| 17 | | | | | | | | | | | | |
| G719A | 48# | | | | 57* | | 47.4* | 28.6* | −1.46* | −1.34* | no* | no* |
| 16 | | | | | | | | | | | | |

| | | | | | | | | | |
|---|---|---|---|---|---|---|---|---|---|
| Accepted values for the | 16-25 | <100 bp | ΔTm | 40- | >25% | >-4 | >−3 | no | no |
| parameters in analysis | bp | | with | 60% | | Kcal/ | Kcal/ | | |
| (paired WT primer) | | | paired | | | mol | mol | | |
| | | | primer | | | | | | |
| | | | <5° C. | | | | | | |

Thermodynamic properties of the EGFR G719 paired wild-type primer previously validated in section VI.).
Accepted values (considered as Tm differences <5° C. between proposed primer candidates) are shown with “*”.
Non-acceptable values are shown with “#”.
In the eventuality that one of the parameters in analysis falls outside the accepted range indicated in the last row the thermodynamic analysis is discontinued and the primer is discarded.

The following combinations were found, that met the thermodynamic criteria required:
G719A 20+G719 P19
G719A 19+G719 P19
G719A 18+G719 P19.

### Blat Alignment of the Validated Primer Pairs in Conventional Sense

Blat alignments (Human GRCh38/hg38) for all the PCR amplicons generated by the combinations of primers meeting the thermodynamic criteria were performed.

All primer pairs produced just one single amplicon at the expected chromosomal position. No other homologies are reported.

VII3) Oligoblocker Design for the EGFR G719 Position

As the EGFR G719A assay is designed to target the same codon of the validated EGFR G719A assay (see chapter VI), it was extremely likely that the validated oligoblocker G719 OB19/2 or the other thermodynamically validated oligoblocker candidate G719 OB18/2 would work fine in combination with the primer candidates proposed in section VII.1/VII.2. A thermodynamic analysis was performed to exclude the presence of hetero-dimers with all primer candidates.

VII4) Thermodynamic Analysis on All Candidate Primers and Oligoblockers

The following heterodimers combinations were considered for all the primers/oligoblocker candidates that met the thermodynamic criteria described in the previous sections:
Allele-specific primer+paired WT primer
Allele-specific primer+oligoblocker
Paired WT primer+oligoblocker.

The results are presented in Table 47.

TABLE 47
Evaluation of heterodimers presence for all oligonucleotide candidates
evaluated for the allele-specific EGFR G719A system.

| Oligonucleotide combination | ΔG (Kcal/mol) | Validated? Y/N |
|---|---|---|
| G719A 18 + G719 P19 | −3.41 | Y |
| G719A 18 + G719 OB18/2 | −0.16 | Y |
| G719A 18 + G719 OB19/2 | −1.41 | Y |
| G719A 19 + G719 P19 | −3.41 | Y |
| G719A 19 + G719 OB19/2 | −1.41 | Y |
| G719A 19 + G719 OB18/2 | −0.16 | Y |
| G719A 20 + G719 P19 | −3.41 | Y |
| G719A 20 + G719 OB19/2 | −1.41 | Y |
| G719A 20 + G719 OB18/2 | 0.16 | Y |
| Accepted values for the | >−4 Kcal/mol | Y |
| parameters in analysis | | |

The results show all oligonucleotide candidates meet the thermodynamic criteria required to ensure absence of non-specific amplification and to improve the amplification efficiency.

VII5) Design of the EGFR G719A Wild-Type Set

As the EGFR G719A assay is designed to target the same codon of the validated EGFR G719A assay (see chapter VI), the validated wild-type EGFR G719 Wild-type assay had to respect all constraints in terms of distance from the mutation-specific primer set to be validated. The WT primer combination G719 WT L+G719 WT R was thus used for the in-vitro validation of the EGFR G719A IntPlex® assay.

VII6) In Vitro Validation of the EGFR G719A Primer Pairs

### G719A 18+G719 P19+G719 OB19/2

### Specificity

The specificity of the combination was tested. The results showed one peak-only, indicating the specificity criteria are met.

### Non-Specificity

A substantial non-specificity was observed when using this oligonucleotide combination. The QC criteria for non-specificity are not met. This combination is thus not optimal.

### G719A 18+G719 P19+G719 OB18/2

### Specificity

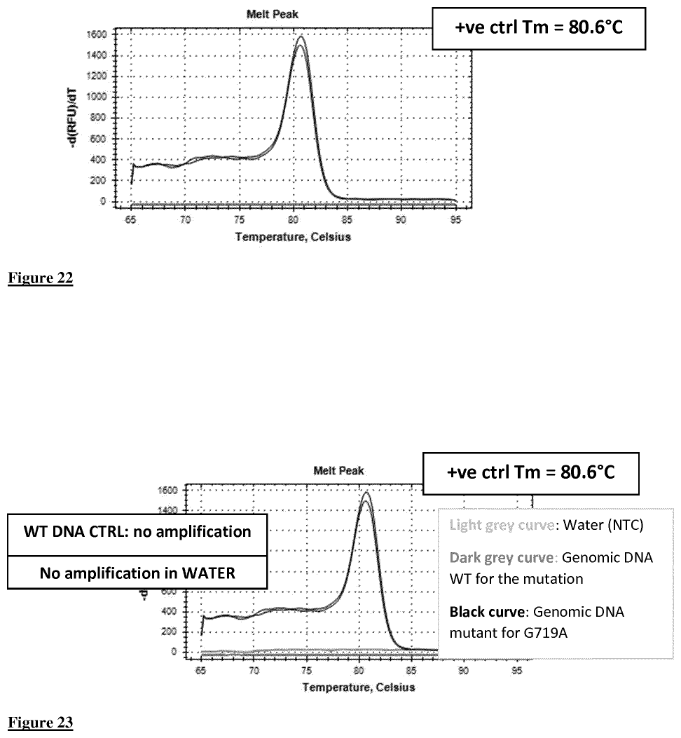

The specificity of this combination was tested. The results are presented in FIG. 22. They showed one peak-only, indicating the specificity criteria are met.

### Non-Specificity

Melting curves of genomic DNA WT for the G719A mutation were compared to melting curves of commercial DNA mutant for G719A using this oligonucleotide combination (see FIG. 23). There was not any non-specificity observed. This oligonucleotide combination was thus evaluated for efficiency.

### Efficiency

The efficiency analysis for the mutation-specific set comprised of G719A 18+G719 P19+G719 OB18/2 was performed in conjunction with the proposed combination for the G719 WT primer set. The results are presented in Table 48.

TABLE 48
Demonstration of the efficiency of the oligonucleotide system for the detection of
EGFR G719A.

| | | | | [DNA] | Tm specific? | | |
|---|---|---|---|---|---|---|---|
| Target | Content | Sample | Cq | ng/μl | (Y/N) | Mean | mA % |
| G719 18 + | Neg | G719S | N/A | N/A | N | | |
| G719 P19 | control | positive | N/A | N/A | N | | |
| G719 OB18/2 | | control | | | | | |
| | NTC | Water | N/A | N/A | N | | |
| | | | N/A | N/A | N | | |
| | Positive | G719A | 27.62 | 0.18 | Y | 0.185 | 60.66% |
| | control | 50% | 27.72 | 0.19 | Y | | |
| G719 WT | | positive | 27.28 | 0.26 | Y | 0.305 | |
| Assay | | control | 27.1 | 0.35 | Y | | |

The observed mutant allele frequency of the G719A mutation-specific system on the G719 WT system is 60.66%, close to expected 50% reported by the supplier of the positive control used for the experiment. The efficiency is thus appropriate and this oligonucleotide combination was further evaluated for sensitivity.

### Sensitivity

The results are presented in Table 49.

TABLE 49
Demonstration of the high sensitivity of IntPlex ® system in analysis for
the detection of the EGFR G719A point mutation. Serial dilutions of a
commercial DNA control positive for the G719A mutation at expected
mutational load of 0.25 ng/μl were performed, up to 1/1000.

| | | [DNA] | | | |
|---|---|---|---|---|---|
| | | (ng/μl) | Tm | | theoretical |
| | Cq | extract | specificity | MEAN | level |
| C+ G719A0.25 | 27.62 | 0.20450 | Y | 0.19750 | 0.25 |
| | 27.72 | 0.19050 | Y | | |
| C+ G719A1/10 | 31.14 | 0.01629 | Y | 0.01587 | 0.025 |
| | 31.21 | 0.01544 | Y | | |
| C+ G719A1/1000 | 35.05 | 0.00097 | Y | 0.00073 | 0.00025 |
| | 35.92 | 0.00052 | Y | | |
| | 35.53 | 0.00069 | Y | | |
| | N/A | N/A | | | |
| | N/A | N/A | | | |
| | N/A | N/A | | | |

The system can detect the G719A mutation down to a frequency of 0.5 pg/μl. The sensitivity is thus very high.

VII7) Conclusions

Our method presents high specificity, efficiency and sensitivity. Among the tested primer combinations, the best system to detect the EGFR G719A mutation is comprised of the mutation-specific set G719A 18+G719 P19+G719 OB18/2 and of the wild-type set G719 WT L+G719 WT R.

Other oligonucleotide combinations that allow efficient detection include:
G719A 19+G719 P19+G719 OB18/2
G719A 19+G719 P19+G719 OB19/2
G719A 20+G719 P19+G719 OB18/2
G719A 20+G719 P19+G719 OB19/2.

### VIII) DEL19

EGFR exon 19 deletions are in-frame deletions occurring within exon 19, which encodes part of the kinase domain. The most frequent deletion in EGFR exon 19 is Δ(E746-A750) resulting from a 15 nucleotide deletion event. The nucleotidic nomenclature of EGFR E746-A750 deletion is c:2235-2249. The 15 nucleotides from 2235 to 2249 on the coding DNA reference sequence are deleted. The inventors have designed specific sets of primers allowing improved detection of the presence (or absence, or frequency) of such a mutation from a sample containing cfNA.

VIII1) Strategy for Detecting Deletion Mutations

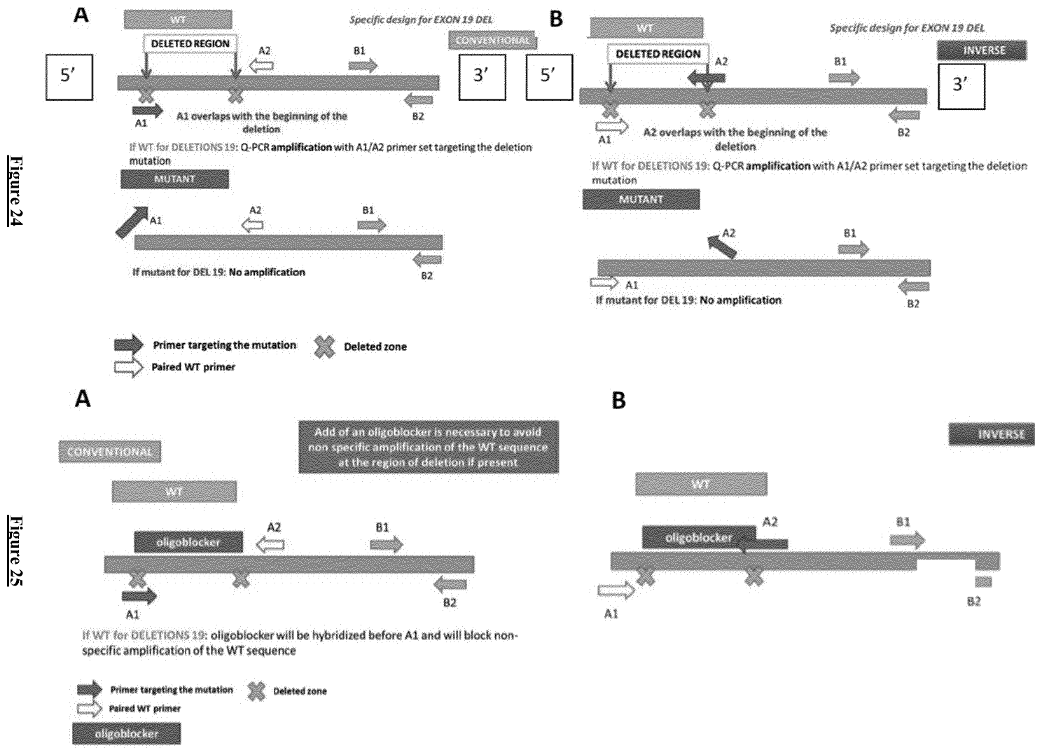

Strategy for the qualitative detection of the deletions is described in FIG. 24.

As previously described, the addition of an oligoblocker allows avoiding non-specific amplification of WT DNA sequences. The strategy for optimal positioning of the oligoblocker is described in FIG. 25.

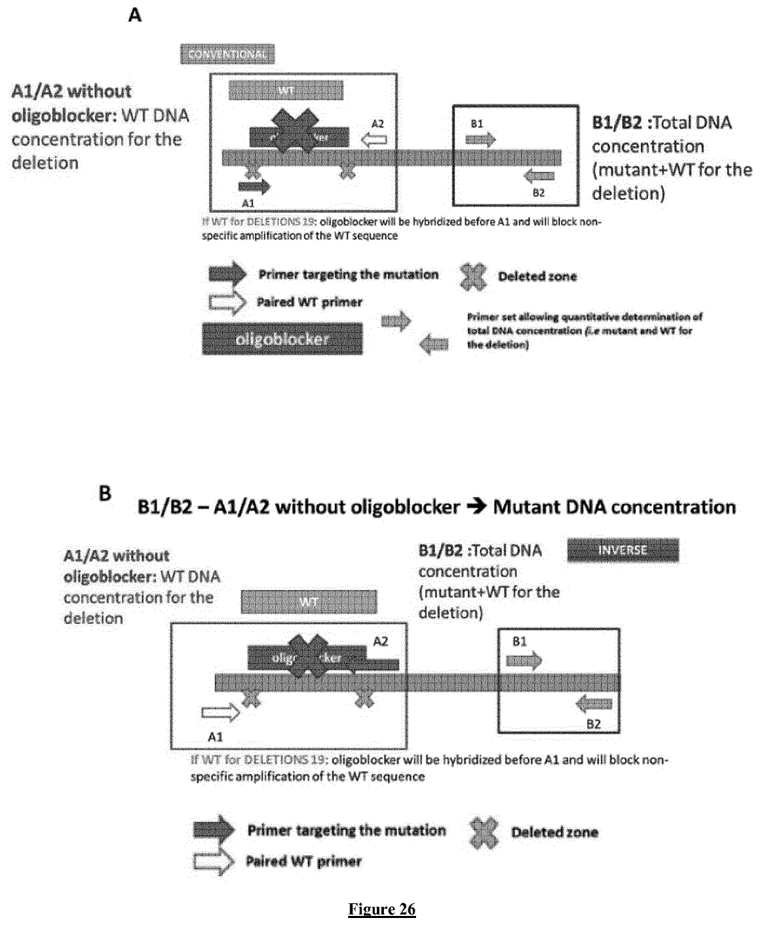

The system presented here allows highly specific and sensitive qualitative detection of the deletion. For quantitative detection, the total DNA concentration can be determined using a B1/B2 WT primer set and by measuring the difference between the total DNA concentration determined with B1/B2 primer set to the WT DNA concentration determined using A1/A2 primer set (FIG. 26).

VIII2) Design of the Allele-Specific EGFR DEL19 Primer

The allele-specific primers generated and considered in the design of this invention and their thermodynamic characteristics are listed in Table 50.

TABLE 50

| | | | | | | | |
|---|---|---|---|---|---|---|---|
| Primer | | | Tm | | GC % | self- | |
| name | Sequence | Length | (° C.) | GC % | tail | dimers | Hairpins |

Conventional configuration

| | | | | | | | |
|---|---|---|---|---|---|---|---|
| DEL19 6N | CCCGTCGCTATC | 20* | 60^{§} | ^{§} | ^{§} | ^{§} | ^{§} |
| | AAGGAATT (SEQ | | | | | | |
| | ID NO: 173) | | | | | | |
| DEL19 | ATCAAGGAATTA | 20* | 54* | 35^{#} | 57.1* | −2.16* | none* |
| 15N | AGAGAAGC (SEQ | | | | | | |
| | ID NO: 174) | | | | | | |
| DEL19 | AAGGAATTAAG | 19* | 50* | 31.6^{#} | 42.9* | −2.16* | none* |
| 17N | AGAAGCAA (SEQ | | | | | | |
| | ID NO: 40) | | | | | | |

Inverse configuration

| | | | | | | | |
|---|---|---|---|---|---|---|---|
| DEL19 | CTTTCGGAGATG | 20 | 58^{§} | ^{§} | ^{§} | ^{§} | ^{§} |
| i6N | TTGCTTCT (SEQ | | | | | | |
| | ID NO: 175) | | | | | | |
| DEL 19 | ATGTTGCTTCTC | 20 | 54* | 35^{#} | 28.6^{#} | −2.16* | none* |
| i15N | TTAATTCC (SEQ | | | | | | |
| | ID NO: 176) | | | | | | |
| DEL19 | TTGCTTCTCTTA | 19 | 50* | 31.6^{#} | 28.6^{#} | −2.16* | none* |
| i17N | ATTCCTT (SEQ | | | | | | |
| | ID NO: 177) | | | | | | |

| | | | | | | |
|---|---|---|---|---|---|---|
| Accepted values for the | 16-22 | 45- | 40- | >25% | >−4 | >−3 |
| parameters in analysis | bp | 55° C. | 60% | | Kcal/ | Kcal/ |
| (allele-specific primer | | | | | mol | mol |
| shown in bold) | | | | | | |

List of all the EGFR DEL19 allele-specific primers considered in the design of this invention and their thermodynamic characteristics.
Note that the nomenclature “N” refers to the number of nucleotides overlapping the expected deletion Δ(E746-A750).
Accepted values are shown with “*”.
Borderline values are shown with “^{#}” and non-acceptable values are shown with “^{§}”.
In the eventuality that one of the parameters in analysis falls outside the accepted range indicated in the last row the thermodynamic analysis is discontinued and the primer is discarded.

VIII3) Paired Wild-Type Primer

Results Obtained with Primer 3

All allele-specific primers that met our criteria (see Table 50) were used on Primer3 to try to find a compatible paired primer. No valid output results were generated applying the suggested parameters.

### Manually Generated Primer Pair Candidates

In view of the unsuccessful generation of a paired primer using conventional tools, we designed such primers manually. All designed primers are listed below, with their characteristics.

TABLE 51

| | | | | | | | | | | | | Off |
|---|---|---|---|---|---|---|---|---|---|---|---|---|
| | Allele- | | | | | | | | | | | target |
| Allele- | specific | | Paired WT | | | | | | | | | with wt |
| specific | Primer | Paired WT | primer | | Amplicon | Tm | | GC% | self- | Hair- | Off | allele- |
| Primer | Tm | primer name | sequence | Length | size | (° C.) | GC % | tail | dimers | pins | target? | specific? |
| DEL19 | 54* | DEL19 P19 | ACTCACATCG | 19 | 60* | 56* | 47.4* | 42.9* | −3.55* | −0.16* | no* | no* |
| 15N | | | AGGATTTCC | | | | | | | | | |
| DEL19 | 50^{#} | | (SEQ ID NO: | | 57* | | | | | | | |
| 17N | | | 41) | | | | | | | | | |

| | | | | | | | | | |
|---|---|---|---|---|---|---|---|---|---|
| Accepted values for the parameters | 16-25 | <100 bp | ΔTm | 40- | >25% | >−4 | >−3 | no | no |
| in analysis (paired WT primer) | bp | | with | 60% | | Kcal/ | Kcal/ | | |
| | | | paired | | | mol | mol | | |
| | | | primer | | | | | | |
| | | | <5° C. | | | | | | |

List of the potential EGFR DEL19 paired WT- primers in conventional configuration manually generated and considered in the design of this invention and their thermodynamic characteristics.
Tm are calculated using the Oligoanalyzer ® software.
Accepted values are shown with “*” and borderline values are shown with “^{#}”.
The results show DEL19 P19 represents an optimal candidate for the assay in conventional configuration.

### Blat Alignment of the Validated Primer Pairs in Conventional Sense

Blat alignments (Human GRCh38/hg38) were performed. They showed the selected optimal primer pairs produced just one single amplicon at the expected chromosomal position.

VIII4) Oligoblocker Design for the EGFR DEL19 Position

We generated several oligoblockers and verified their characteristics, as shown in Table 52.

TABLE 52

| Oligo- | | | | | | | |
|---|---|---|---|---|---|---|---|
| blocker | | | Tm | | GC % | self- | |
| name | Sequence | Length | (° C.) | GC % | tail | dimers | Hairpins |
| DEL19 | TCAAGGAATTA | 23 | 62* | 34.8^{§} | ^{§} | ^{§} | ^{§} |
| OB23 | AGAGAAGCAAC | | | | | | |
| | A (SEQ ID NO: | | | | | | |
| | 178) | | | | | | |
| DEL19 | CAAGGAATTAA | 22 | 60* | 36.8^{§} | ^{§} | ^{§} | ^{§} |
| OB22 | GAGAAGCAACA | | | | | | |
| | (SEQ ID NO: 179) | | | | | | |
| DEL19 | AAGGAATTAAG | 21 | 56^{§} | ^{§} | ^{§} | ^{§} | ^{§} |
| OB21 | AGAAGCAACA | | | | | | |
| | (SEQ ID NO: 180) | | | | | | |
| DEL19 | AGGAATTAAGA | 20 | 54^{§} | ^{§} | ^{§} | ^{§} | ^{§} |
| OB20 | GAAGCAACA | | | | | | |
| | (SEQ ID NO: 181) | | | | | | |
| DEL19 | GGAATTAAGAG | 19 | 52^{§} | ^{§} | ^{§} | ^{§} | ^{§} |
| OB19 | AAGCAACA | | | | | | |
| | (SEQ ID NO: 182) | | | | | | |
| DEL19 | TCAAGGAATTA | 22 | 60* | 36.4^{§} | ^{§} | ^{§} | ^{§} |
| OB22/2 | AGAGAAGCAA | | | | | | |
| | (SEQ ID NO: 183) | | | | | | |
| DEL19 | TCAAGGAATTA | 21 | 56^{§} | ^{§} | ^{§} | ^{§} | ^{§} |
| OB21/2 | AGAGAAGCA | | | | | | |
| | (SEQ ID NO: 184) | | | | | | |
| DEL19 | TCAAGGAATTA | 20 | 54^{§} | ^{§} | ^{§} | ^{§} | ^{§} |
| OB20/2 | AGAGAAGC | | | | | | |
| | (SEQ ID NO: 185) | | | | | | |
| DEL19 | TCAAGGAATTA | 19 | 52^{§} | ^{§} | ^{§} | ^{§} | ^{§} |
| OB19/2 | AGAGAAG (SEQ | | | | | | |
| | ID NO: 186) | | | | | | |
| DEL19 | GGAATTAAGAG | 23 | 64^{§} | ^{§} | ^{§} | ^{§} | ^{§} |
| OB23/3 | AAGCAACATCT | | | | | | |
| | C (SEQ ID NO: | | | | | | |
| | 187) | | | | | | |
| DEL19 | GGAATTAAGAG | 22 | 60* | 36.4^{§} | ^{§} | ^{§} | ^{§} |
| OB22/3 | AAGCAACATCT | | | | | | |
| | (SEQ ID NO: 188) | | | | | | |
| DEL19 | GGAATTAAGAG | 21 | 58* | 38.1^{#} | 42.9* | 2.16* | none* |
| OB21/3 | AAGCAACATC | | | | | | |
| | (SEQ ID NO: 42) | | | | | | |
| DEL19 | GGAATTAAGAG | 20 | 54^{§} | ^{§} | ^{§} | ^{§} | ^{§} |
| OB20/3 | AAGCAACAT | | | | | | |
| | (SEQ ID NO: 189) | | | | | | |
| DEL19 | GGAATTAAGAG | 19 | 52^{§} | ^{§} | ^{§} | ^{§} | ^{§} |
| OB19/3 | AAGCAACA | | | | | | |
| | (SEQ ID NO: 190) | | | | | | |

| | | | | | | |
|---|---|---|---|---|---|---|
| Accepted values for the | 19- | ~60° C. | 40- | >25% | >−4 | >−3 |
| parameters in analysis | 23 bp | | 60% | | Kcal/ | Kcal/ |
| (Oligoblocker shown in | | | | | mol | mol |
| bold) | | | | | | |

List of all potential oligoblockers for EGFR DEL19, in conventional configuration, considered in the design of this invention and their thermodynamic characteristics.
Tm are calculated using the Oligoanalyzer ® software.
Accepted values are shown with “*”.
Borderline values are shown with “^{#}” and non-acceptable values are shown with “^{§}”.
In the eventuality that one of the parameters in analysis falls outside the accepted range indicated in the last row the thermodynamic analysis is discontinued and the oligoblocker is discarded.

The best oligoblocker appeared to be DEL 19 OB21/3 and it was thus selected for all further thermodynamic analyses.

VIII5) Thermodynamic Analysis on All Candidate Primers and Oligoblockers

The following heterodimers combinations were considered for all the primers/oligoblocker candidates that met the thermodynamic criteria:
Allele-specific primer+paired WT primer
Allele-specific primer+oligoblocker
Paired WT primer+oligoblocker.

The results are presented in Table 53.

TABLE 53
Evaluation of heterodimers presence for all oligonucleotide
candidates evaluated for the allele-specific system.

| | ΔG | Validated |
|---|---|---|
| Oligonucleotide combination | (Kcal/mol) | Y/N |
| DEL19 15N + DEL19 P19 | −3.38 | Y |
| DEL19 15N + DEL19 0B21/3 | −2.16 | Y |
| DEL19 17N + DEL19 P19 | 3.38 | Y |
| DEL19 17N + DEL19 OB21/3 | −2.16 | Y |
| DEL19 P19 + DEL19 OB21/3 | 3.38 | Y |
| Accepted values for the | >−4 Kcal/mol | Y |
| parameters in analysis | | |

The results show all oligonucleotide candidates meet the thermodynamic criteria required to ensure absence of non-specific amplification and to improve the amplification efficiency.

VIII6) Design of the DEL19 Wild-Type Primer Set

The optimal positioning of the DEL19 wild-type primer candidates on the genomic nucleic acid sequence is shown in FIG. 27.

The tested primers are disclosed in Table 54.

TABLE 54

| | | | | | GC % | self- | Hair- | Hetero- | Validated |
|---|---|---|---|---|---|---|---|---|---|
| Primer name | Sequence | Length | Tm (° C.) | GC % | tail | dimers | pins | dimers | (Y/N) |
| DEL19 WT L | CTTAGAGACAGCACTGGC | 18 | 56* | 55.6* | 71.4* | −0.34* | none* | −3* | Y |
| | (SEQ ID NO: 43) | | | | | | | | |
| DEL19 WT R | CAGTAATTGCCTGTTTCC | 18 | 52* | 44.4* | 42.9* | −2.16* | −0.22* | | Y |
| | (SEQ ID NO: 44) | | | | | | | | |

| | | | | | | | | |
|---|---|---|---|---|---|---|---|---|
| Accepted values for the | 16- | 55-60° C. | 40- | >25% | >−4 | >−3 | >−4 | Y |
| parameters in analysis | 22 bp | ΔTm | 60% | | Kcal/ | Kcal/ | Kcal/ | |
| (wild-type primer set) | | with | | | mol | mol | mol | |
| | | paired | | | | | | |
| | | primer | | | | | | |
| | | <5° C. | | | | | | |

Evaluation of all thermodynamic parameters considered to select appropriate oligonucleotides for the EGFR DEL19 wild-type set.
Accepted values are shown with “*”.

The primers exhibited the required characteristics.

### Blat Alignment of the Validated Primer Pairs in Conventional Sense

Blat alignments (Human GRCh38/hg38) were performed for the PCR amplicons generated by the combinations of primers.

The Blat local alignment highlighted the presence of a secondary homology region. Nevertheless, by analyzing the structure and extension of the homology region reported on chr3, we concluded that it would produce just a partial annealing region for the DEL19 WT L primer but, as no homology is present in the target region of the DEL19 WT R primer, no secondary product would be produced.

The oligonucleotide candidates of the DEL19 WT set thus met the thermodynamic criteria required to ensure absence of non-specific amplification and to improve the amplification efficiency.

VIII7) In Vitro Validation of the EGFR DEL19 Primers

### DEL19 15N+DEL19 P19+DEL19 OB21/3

### Specificity and Non-Specificity

A substantial non-specificity was observed when using this oligonucleotide combination.

### DEL19 17N+DEL19 P19+DEL19 OB21/3

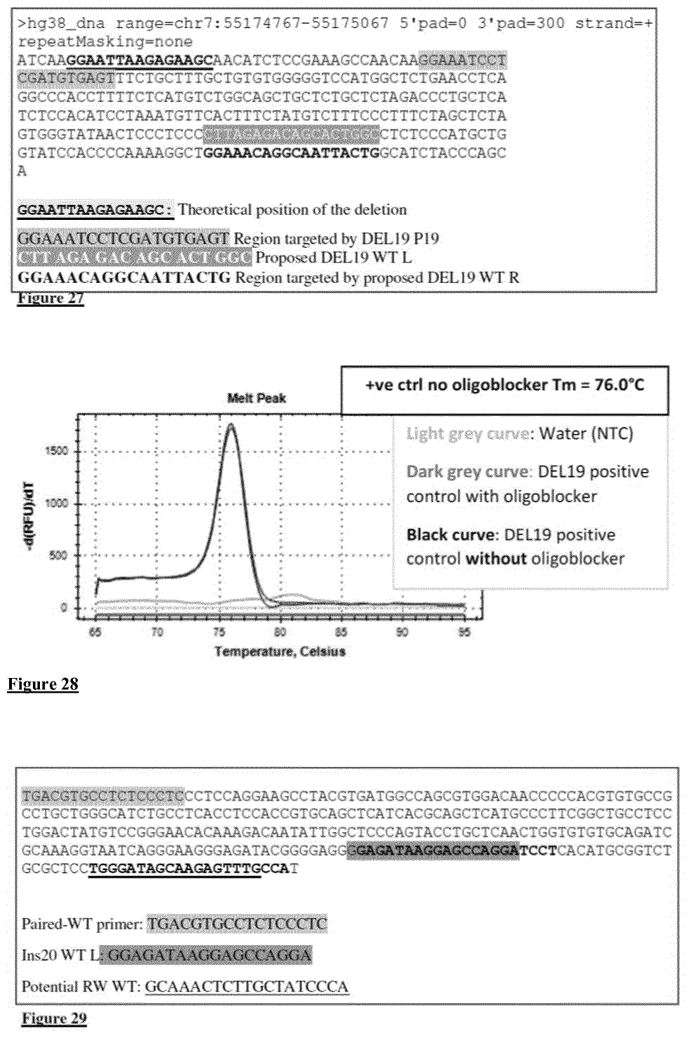

### Specificity and Non-Specificity (see FIG. 28)

This oligonucleotide combination is highly specific.

### Efficiency

The efficiency analysis for the mutation-specific set comprised of DEL19 17N+DEL19 P19+DEL19 OB21/3 was performed in conjunction with the proposed system for the DEL19 WT primer set.

TABLE 55
Demonstration of the efficiency of the oligonucleotide system for the detection of
EGFR DEL19 Δ(E746-A750).

| | | | | [DNA] | | |
|---|---|---|---|---|---|---|
| Target | Content | Sample | Cq | ng/μl | Mean | mA % |
| DEL19 17N + | Neg | T790M | N/A | N/A | | |
| DEL19 P19 + | control | 50% + ve ctrl | | N/A | | |
| DEL19 OB21/3 | NTC | Water | N/A | N/A | | |
| | | | 3765 | 0.00008 | | |
| | Positive | DEL19 | 37.17 | 0.00012 | 0.00012 | |
| | control | 50% + ve ctrl | 37.15 | 0.00012 | | |
| WT Assay | | | 25.16 | 1.152 | 1.142 | |
| | | | 25.18 | 1.131 | | |
| DEL19 17N + | Positive | DEL19 | 25.82 | 0.6894 | 0.67385 | 47% |
| DEL19 P19 + | control | 50% + ve ctrl | 25.88 | 0.6583 | | |
| DEL19 | | | | | | |

The observed mutant allele frequency of the EGFR DEL19-specific system on the DEL19 WT system is 47%, close to expected 50% reported by the supplier of the positive control used for the experiment. The method thus provides high efficiency.

### Sensitivity

TABLE 56
Demonstration of the high sensitivity of the method for the
detection of the EGFR Δ(E746-A750). Serial dilutions of
a commercial DNA control positive for the
EGFR Δ(E746-A750) were performed, up to 1/10000.

| | | | [DNA] | |
|---|---|---|---|---|
| | | | (ng/μ1) | |
| Target | Sample | Cq | extract | mean |
| DEL 19 A1/A2-Blocker | C + DEL19 | 27.63 | 0.1583 | 0.1594 |
| | | 27.61 | 0.1604 | |
| | C + DEL19 1/10 | 30.86 | 0.0142 | 0.0145 |
| | | 30.8 | 0.0149 | |
| | C + DEL19 1/100 | 33.72 | 0.0017 | 0.0017 |
| | C + DEL19 1/1000 | 37.66 | 0.0001 | 0.0001 |
| | | N/A | N/A | |
| | C + DEL19 1/10000 | N/A | N/A | N/A |
| | | N/A | N/A | |

The method can detect the EGFR deletion Δ(E746-A750) down to a frequency of 0.1 pg/μl. The sensitivity is thus very high.

VIII8) Conclusions

The results here presented confirm that the method allows detection of DEL19 with high specificity, efficiency and sensitivity. Optimal primers include the mutation-specific set DEL19 17N+DEL19 P19 with and without oligoblocker DEL19 OB21/3 and of the wild-type set DEL19 WT L+DEL19 WT R.

IX) EGFR Exon 20 Insertions (Ins20) V769-D770 insASV

EGFR exon 20 insertions are in-frame insertions occurring within exon 20. Most EGFR exon 20 insertions occur in between amino acids 767 to 774 of exon 20, within the loop that follows the C-helix of the kinase domain of EGFR (Yasuda, Kobayashi, and Costa 2012). In terms of coding DNA reference sequence refers to c: 2302-2320. Insertions can vary from 3 to 12 nucleotides, with the most common being EGFR V769-D770 insASV (c.2308_2309insGCCAGCGTG).

The inventors have designed specific sets of primers allowing improved detection of the presence (or absence, or frequency) of such a mutation from a sample containing cfNA.

IX1) Design of the Allele-Specific EGFR Ins20 Primer

The allele-specific primers generated and considered in the design of this invention and their thermodynamic characteristics are listed in Table 57.

The design in conventional configuration was strongly unflavored: the presence of a region of self-dimerization impaired any potential primer design in conventional sense spanning the insertion junction from the gene 5′. Only allele-specific primer candidates in inverse configuration were thus considered for all thermodynamic analyses.

TABLE 57

| | | | | | | | |
|---|---|---|---|---|---|---|---|
| Primer | | | Tm | | GC% | self- | |
| name | Sequence | Length | (° C.) | GC % | tail | dimers | Hairpins |

Inverse configuration

| | | | | | | | |
|---|---|---|---|---|---|---|---|
| INS20 | GGTTGTCCACGC | 16 | 54* | 68.8^{#} | 85.7* | −1.81* | −1.69* |
| AVS i16 | TGGC (SEQ ID | | | | | | |
| | NO: 45) | | | | | | |
| INS20 | GGGTTGTCCACG | 16 | 54* | 68.8^{#} | 71.4* | −1.81* | −1.69* |
| AVS i16/2 | CTGG (SEQ ID | | | | | | |
| | NO: 46) | | | | | | |
| INS20 | GGGGTTGTCCAC | 16 | 54* | 68.8^{#} | 71.4* | none* | none* |
| AVS i16/3 | GCTG (SEQ ID | | | | | | |
| | NO: 47) | | | | | | |
| INS20 | GGGGGTTGTCCA | 16 | 54* | 68.8^{#} | 71.4* | none* | none* |
| AVS i16/4 | CGCT (SEQ ID | | | | | | |
| | NO: 48) | | | | | | |
| INS20 | TGGGGGTTGTCC | 16 | 54* | 68.8^{#} | 71.4* | −1.81* | −1.69* |
| AVS i16/5 | ACGC (SEQ ID | | | | | | |
| | NO: 49) | | | | | | |

| | | | | | | |
|---|---|---|---|---|---|---|
| Accepted values for the | 16-22 | 45- | 40- | >25% | >−4 | >−3 |
| parameters in analysis (allele- | bp | 55° C. | 60% | | Kcal/ | Kcal/ |
| specific primer shown in bold) | | | | | mol | mol |

List of all the EGFR Ins20 AVS allele-specific primers in inverse configuration considered in the design of this invention and their thermodynamic characteristics.
Accepted values are shown with “*” and borderline values are shown with “^{#}”.
In the eventuality that one of the parameters in analysis falls outside the accepted range indicated in the last row the thermodynamic analysis is discontinued and the primer is discarded.

Because of the very high GC % of the area, longer allele-specific primers were not considered as the Tm would have been too close to 60° C. (Optimal annealing temperature for the oligoblocker).

All primer pairs do have the same high GC % but there were no alternative options. According to Oligoanalyzer all primers possessed similar thermodynamic characteristics. Nevertheless, as general precaution when designing primers, it is recommended to avoid G quadraplexes. This rule was applied when analyzing allele-specific primer with similar thermodynamic characteristics.

IX2) Paired Wild-Type Primer

Results Obtained with Primer 3

All allele-specific primers that met our Icriteria (see Table 57) were used on Primer3 to try to find a compatible paired primer. Results are summarized in Table 58.

TABLE 58
Ins20 AVS paired WT- primer candidate produced by Primer 3 and considered in the design of
this invention. Note that Tm are calculated following the recommended Primer3 algorithm.
Accepted values are shown with “*”. Non-acceptable values are shown with “§”. In the
eventuality that one of the parameters in analysis falls outside the accepted range
indicated in the last row the thermodynamic analysis is discontinued and the primer is
discarded.

| | | | | | | | | | | | | Off |
|---|---|---|---|---|---|---|---|---|---|---|---|---|
| | Allele- | Paired | Paired | | | | | | | | | target |
| Allele- | specific | WT | WT | | | | | | | | | with wt |
| specific | Primer Tm | primer | primer | | Amplicon | Tm | | GC % | self- | Hair- | Off | allele- |
| Primer | (Primer 3) | name | sequence | Length | size | (° C.) | GC % | tail | dimers | pins | target? | specific? |
| INS20 | 59.74 | Ins20 | CACACTGA | 19 | 69* | 60.47^{§} | § | § | § | § | § | § |
| AVSi16 | | iP19 | CGTGCCTC | | | | | | | | | |
| | | | TCC (SEQ | | | | | | | | | |
| | | | ID NO: | | | | | | | | | |
| | | | 191) | | | | | | | | | |

| | | | | | | | | | |
|---|---|---|---|---|---|---|---|---|---|
| Accepted values for the | 16-25 bp | <100 bp | ΔTm | 40-60% | >25% | >−4 | >−3 | no | no |
| parameters in analysis | | | with | | | Kcal/mol | Kcal/mol | | |
| (paired WT primer) | | | paired | | | | | | |
| | | | primer | | | | | | |
| | | | <5° C. | | | | | | |

From the results presented in Table 58 it was not possible to select a suitable candidate as the only output presented too high melting temperature.

By manually reducing the size of the primer candidate proposed by Primer3 a better fit could be reached. The paired-wild type primer was manually designed.

### Manually Generated Primer Pair Candidates

In view of the unsuccessful generation of a paired primer using conventional tools, we designed such primers manually. All designed primers are listed in Table 59, with their characteristics.

TABLE 59
List of all potential Ins20 AVS paired WT- primers in conventional configuration manually
generated and considered in the design of this invention and their thermodynamic
characteristics. Note that Tm are calculated using the Oligoanalyzere software. Accepted
values are shown with “*” and borderline values are shown with “#”.

| | | | | | | | | | | | | Off |
|---|---|---|---|---|---|---|---|---|---|---|---|---|
| | Allele- | Paired | | | | | | | | | | target |
| Allele- | specific | WT | Paired WT | | | | | | | | | with wt |
| specific | Primer | primer | primer | | Amplicon | Tm | | GC % | self- | Hair- | Off | allele- |
| Primer | Tm | name | sequence | Length | size | (° C.) | GC % | tail | dimers | pins | target? | specific? |
| INS20 | 54 | Ins20 | ACACTGACG | 18 | 68* | 58* | 61.1^{#} | 71.4* | −3.09* | 0.04* | N* | N* |
| AVSi16 | | iP18 | TGCCTCTCC | | | | | | | | | |
| | | | (SEQ ID | | | | | | | | | |
| | | | NO: 50) | | | | | | | | | |
| | | Ins20 | ACACTGACG | 17 | 68* | 54* | 58.80* | 71.4* | −3.09* | 0.04* | N* | N* |
| | | iP17 | TGCCTCTC | | | | | | | | | |
| | | | (SEQ ID | | | | | | | | | |
| | | | NO: 51) | | | | | | | | | |
| | | Ins20 | TGACGTGCC | 17 | 64* | 56* | 64.70^{#} | 71.4* | −3.09* | none* | N* | N* |
| | | iP17/2 | TCTCCCTC | | | | | | | | | |
| | | | (SEQ ID | | | | | | | | | |
| | | | NO: 52) | | | | | | | | | |

| | | | | | | | | | |
|---|---|---|---|---|---|---|---|---|---|
| Accepted values for the | 16-25 bp | <100 bp | ΔTm | 40-60% | >25% | >−4 | >−3 | no | no |
| parameters in analysis | | | with | | | Kcal/mol | Kcal/mol | | |
| (paired WT primer) | | | paired | | | | | | |
| | | | primer | | | | | | |
| | | | <5° C. | | | | | | |

All WT primer candidates represented suitable choices for the assay in inverse configuration. The GC % was quite high but as it is region-dependent it could not be lowered further.

### Blat Alignment of the Validated Primer Pairs in Inverse Configuration

Blat alignments (Human GRCh38/hg38) for all the PCR amplicons generated all combinations of primers were performed. The primer combinations provided one principal hit on the EGFR gene. The percentage of identity is <100% as the presence of the insertion would add 9 nucleotides not present in the reference sequence. All secondary hits did not span any primer landing site.

All primer pair combinations produced just one single amplicon at the expected chromosomal position.

IX3) Oligoblocker Design for the Allele-Specific EGFR Ins20 AVS Position

We generated several oligoblockers and verified their characteristics, as shown in Table 60.

TABLE 60
List of all potential oligoblockers for the EGFR Ins20 around
the typical junction for the AVS insertion, in inverse
configuration, considered in the design of this invention and
their thermodynamic characteristics. Tm are calculated using
the Oligoanalyzer ® software. Accepted values are shown with “*”.
Borderline values are shown with “#” and non-acceptable values
are shown with “§”. In the eventuality that one of the parameters
in analysis falls outside the accepted range indicated in the
last row the thermodynamic analysis is discontinued and the
oligoblocker is discarded.

| Oligoblocker | | | Tm | | GC % | self- | Hair- |
|---|---|---|---|---|---|---|---|
| name | Sequence | Length | (° C.) | GC % | tail | dimers | pins |
| Ins20AVS | CACGTGGGGGTT | 23 | 78^{§} | § | § | § | § |
| i0B23 | GTCCACGCTGG | | | | | | |
| | (SEQ ID NO: | | | | | | |
| | 136) | | | | | | |
| Ins20AVS | ACGTGGGGGTTG | 22 | 74^{§} | § | § | § | § |
| i0B22 | TCCACGCTGG | | | | | | |
| | (SEQ ID NO: | | | | | | |
| | 137) | | | | | | |
| Ins20AVS | CGTGGGGGTTGT | 21 | 72^{§} | § | § | § | § |
| i0B21 | CCACGCTGG | | | | | | |
| | (SEQ ID NO: | | | | | | |
| | 138) | | | | | | |
| Ins20AVS | GTGGGGGTTGTC | 20 | 68^{§} | § | § | § | § |
| i0B20 | CACGCTGG | | | | | | |
| | (SEQ ID NO: | | | | | | |
| | 139) | | | | | | |
| Ins20AVS | TGGGGGTTGTCC | 19 | 64^{§} | § | § | § | § |
| i0B19 | ACGCTGG | | | | | | |
| | (SEQ ID NO: | | | | | | |
| | 37) | | | | | | |
| Ins20AVS | TGGGGGTTGTCC | 18^{#} | 60* | 66.74 | 71.4* | −1.81* | −1.69* |
| i0B18 | ACGCTG | | | | | | |
| | (SEQ ID NO: | | | | | | |
| | 53) | | | | | | |

| | | | | | | |
|---|---|---|---|---|---|---|
| Accepted values for the | 19-23 bp | ~60° C. | 40-60% | >25% | >−4 | >−3 |
| parameters in analysis | | | | | Kcal/mol | Kcal/mol |
| (Oligoblocker shown | | | | | | |
| in bold) | | | | | | |

The best oligoblocker appeared to be Ins20AVS iOB 18 that was thus selected for all further thermodynamic analyses.

IX4) Thermodynamic Analysis on All Candidate Primers and Oligoblockers

The following heterodimers combinations were considered for all the primers/oligoblocker candidates that met the thermodynamic criteria:
Allele-specific primer+paired WT primer
Allele-specific primer+oligoblocker
Paired WT primer+oligoblocker.

The results are presented in Table 61.

TABLE 61
Evaluation of heterodimers presence for all oligonucleotide
candidates evaluated for the allele−specific system.

| Oligonucleotide combination | ΔG (Kcal/mol) | Validated? Y/N |
|---|---|---|
| INS20 AVS i16 + Ins20 iP17 | −3.7 | Y |
| INS20 AVS i16 + Ins20 iP17/2 | −3.7 | Y |
| INS20 AVS i16 + Ins20 iP18 | −3.7 | Y |
| INS20 AVS i16 + Ins20AVS i0B18 | −1.81 | Y |
| Ins20 iP17 + Ins20AVS i0B18 | −3.7 | Y |
| Ins20 iP17/2 + Ins20AVS i0B18 | −3.7 | Y |
| Ins20 iP18 + Ins20AVS i0B18 | −3.7 | Y |
| Accepted values for the parameters | >−4 Kcal/mol | Y |
| in analysis | | |

The results show all oligonucleotide candidates meet the thermodynamic criteria required to ensure absence of non-specific amplification and to improve the amplification efficiency.

IX5) Design of the Ins20 Wild-Type Primer Set

The optimal positioning of Ins20 wild-type primer candidates on the genomic nucleic acid sequence is presented in FIG. 29. The tested primers are disclosed in Table 62.

TABLE 62
Evaluation of all thermodynamic parameters considered to select appropriate
oligonucleotides for the Ins20 wild-type set. Accepted values are shown
with “*”. Borderline values are shown with “#”.

| | | | | | GC % | self- | Hair- | Hetero- | Validated |
|---|---|---|---|---|---|---|---|---|---|
| Primer name | Sequence | Length | Tm (° C.) | GC % | tail | dimers | pins | dimers | (Y/N) |
| Ins20 WT L | GGAGATAAGGAGCCAGGA | 18 | 56* | 55.6* | 71.4* | none* | none* | −1.53* | Y |
| | (SEQ ID NO: 54) | | | | | | | | |
| Ins20 WT R | GCAAACTCTTGCTATCCC | 19 | 56* | 47.4* | 42.9* | −3.83* | −3.71^{#} | | Y |
| | A (SEQ ID NO: 55) | | | | | | | | |

| | | | | | | | | |
|---|---|---|---|---|---|---|---|---|
| Accepted values for the | 16-22 bp | 55-60° C. | 40-60% | >25% | >−4 | >−3 | >−4 | Y |
| parameters in analysis | | ΔTm | | | Kcal/mol | Kcal/mol | Kcal/mol | |
| (wild-type primer set) | | with | | | | | | |
| | | paired | | | | | | |
| | | primer | | | | | | |
| | | <5° C. | | | | | | |

The primers exhibit the required characteristics.

### Blat Alignment of the Validated Primer Pairs in Conventional Sense

Blat alignments (Human GRCh38/hg38) were performed for the PCR amplicons generated by all combinations of primers. All primer pairs produced one single amplicon only at the expected chromosomal position. No other homologies were reported.

The oligonucleotide candidates of the EGFR Ins20 WT set met the thermodynamic criteria required to ensure absence of non-specific amplification and to improve the amplification efficiency.

IX6) Conclusions

The results here presented confirmed that the method allows detection of EGFR Ins20 AVS insertion with high specificity, efficiency and sensitivity. Optimal primers include the following combinations:
INS20 AVS i16+Ins20 iP17++Ins20AVS iOB18;
INS20 AVS i16+Ins20 iP17/2++Ins20AVS iOB18;
INS20 AVS i16+Ins20 iP18++Ins20AVS iOB18.

Other suitable combinations include:
INS20 AVS i16/2+Ins20 iP17++Ins20AVS iOB18;
INS20 AVS i16/2+Ins20 iP17/2++Ins20AVS iOB18;
INS20 AVS i16/2+Ins20 iP18++Ins20AVS iOB18;
INS20 AVS i16/3+Ins20 iP17++Ins20AVS iOB18;
INS20 AVS i16/3+Ins20 iP17/2++Ins20AVS iOB18;
INS20 AVS i16/3+Ins20 iP18++Ins20AVS iOB18;
INS20 AVS i16/4+Ins20 iP17++Ins20AVS iOB18;
INS20 AVS i16/4+Ins20 iP17/2++Ins20AVS iOB18;
INS20 AVS i16/4+Ins20 iP18++Ins20AVS iOB18;
INS20 AVS i16/5+Ins20 iP17++Ins20AVS iOB18;
INS20 AVS i16/5+Ins20 iP17/2++Ins20AVS iOB18;
INS20 AVS i16/5+Ins20 iP18++Ins20AVS iOB18.

The following combination of primers can be used as paired wild-type assay for the EGFR Ins20 AVS insertion: Ins20 WT L+Ins20 WT R.

LIST OF SEQUENCES

| SEQ | | | |
|---|---|---|---|
| ID | | | |
| NO: | Name | Sequence | Mutation |
| 1 | T790M 16 | CCGTGCAGCTCATCAT | T790M |
| 2 | T790M 17 | ACCGTGCAGCTCATCAT | |
| 3 | T790 P19 | TATTGTCTTTGTGTTCCCG | |
| 4 | T790 P20 | ATATTGTCTTTGTGTTCCCG | |
| 5 | T790 OB19 | CAGCTCATCACGCAGCTCA | |
| 6 | T790 WT L | CCTGTGCTAGGTCTTTTGC | |
| 7 | T790 WT R | CATGAATGCGATCTTGAGT | |
| 8 | L858R 16 | TCACAGATTTTGGGCG | L858R |
| 9 | L858R 17 | ATCACAGATTTTGGGCG | |
| 10 | L858R 18 | GATCACAGATTTTGGGCG | |
| 11 | L858R 19 | AGATCACAGATTTTGGGCG | |
| 12 | L858 P20 | CTCCTTCTGCATGGTATTCT | |
| 13 | L858 OB19/3 | TCACAGATTTTGGGCTGGC | |
| 14 | L858 OB20/3 | ATCACAGATTTTGGGCTGGC | |
| 15 | L858 WT L | GGTCTCCTGGTAGTGTG | |
| 16 | L858 WT R | TCCTCATTCACTGTCCCA | |
| 17 | L861Q i17 | CTCTTCCGCACCCAGCT | L861Q |
| 18 | L861Q i18 | TCTCTTCCGCACCCAGCT | |
| 19 | L861 iP20 | GTGAAAACACCGCAGCATGT | |
| 20 | L861 iOB19/2 | TCCGCACCCAGCAGTTTGG | |
| 21 | L861 iOB20/2 | TCCGCACCCAGCAGTTTGGC | |
| 22 | L861 WT L | GATGCTCTCCAGACATTCT | |
| 23 | L861 WT R | CTGTTCCCAAAGCAGCTCT | |
| 24 | G719S 19 | AAAAAGATCAAAGTGCTGA | G719S |
| 25 | G719S 20 | CAAAAAGATCAAAGTGCTGA | |
| 26 | G719 P19 | CCAGGGACCTTACCTTATA | |
| 27 | G719 OB18/2 | ATCAAAGTGCTGGGCTCC | |
| 28 | G719 OB19/2 | GATCAAAGTGCTGGGCTCC | |
| 29 | G719 WT L | GTCCTGTGAGACCAATTCA | |
| 30 | G719 WT R | CATGGCAAGGTCAATGGC | |
| 31 | G719A 18 | AAAGATCAAAGTGCTGGC | G719A |
| 32 | G719A 19 | AAAAGATCAAAGTGCTGGC | |
| 33 | G719A 20 | AAAAAGATCAAAGTGCTGGC | |
| 34 | S768I i16 | TGGGGGTTGTCCACGA | S768I |
| 35 | S768I i17 | GTGGGGGTTGTCCACGA | |
| 36 | S768 iP18 | AGCCACACTGACGTGCCT | |
| 37 | S768 iOB19 | TGGGGGTTGTCCACGCTGG | |
| 38 | S768 WT L | GATCGCAAAGGTAATCAGG | |
| 39 | S768 WT R | TCTTGCTATCCCAGGAGC | |
| 40 | DEL19 17N | AAGGAATTAAGAGAAGCAA | DEL19 |
| 41 | DEL19 P19 | ACTCACATCGAGGATTTCC | |
| 42 | DEL19 OB21/3 | GGAATTAAGAGAAGCAACATC | |
| 43 | DEL19 WT L | CTTAGAGACAGCACTGGC | |
| 44 | DEL19 WT R | CAGTAATTGCCTGTTTCC | |
| 45 | INS20 AVS | GGTTGTCCACGCTGGC | INS20 |
| | i16 | | AVS |
| 46 | INS20 AVS | GGGTTGTCCACGCTGG | |
| | i16/2 | | |
| 47 | INS20 AVS | GGGGTTGTCCACGCTG | |
| | i16/3 | | |
| 48 | INS20 AVS | GGGGGTTGTCCACGCT | |
| | i16/4 | | |
| 49 | INS20 AVS | TGGGGGTTGTCCACGC | |
| | i16/5 | | |
| 50 | Ins20 iP18 | ACACTGACGTGCCTCTCC | |
| 51 | Ins20 iP17 | ACACTGACGTGCCTCTC | |
| 52 | Ins20 iP17/2 | TGACGTGCCTCTCCCTC | |
| 53 | Ins20 AVS | TGGGGGTTGTCCACGCTG | |
| | iOB318 | | |
| 54 | Ins20 WT L | GGAGATAAGGAGCCAGGA | |
| 55 | Ins20 WT R | GCAAACTCTTGCTATCCCA | |

1-22. (canceled)

23. A method for determining the presence of a mutation in the EGFR gene in a sample from a subject, the method comprising amplifying, in a sample of a biological fluid from the subject containing cell free nucleic acids, at least a first target sequence from the EGFR gene, the first target sequence containing a site of a mutation on said gene, wherein the first amplified target sequence is 50-95 nt in length and amplification of the first target sequence is conducted with a first set of reagents comprising (i) a mutation-specific primer of 15-25 nt in length, (ii) a paired primer of 15-25 nt in length and (iii) an oligoblocker, wherein the oligoblocker hybridizes specifically to the first target sequence in non-mutated form and blocks polymerase elongation.

24. The method of claim 23, wherein the mutation is a nucleotide substitution and wherein the mutation-specific primer contains the mutated position at one terminal nucleotide.

25. The method of claim 23, wherein the mutation is a deletion and wherein the mutation-specific primer overlaps with the junction region in the gene created by said deletion.

26. The method of claim 23, wherein the mutation is an insertion and wherein the mutation-specific primer overlaps with the junction region in the gene created by said insertion.

27. The method of claim 23, wherein the mutation is selected from L858R, G719A, G719C, G719S, S768I, T790M, L861Q, exon19DELΔ(E746-A750) and exon20INS.

28. The method of claim 27, wherein the mutation is EGFR L858R and wherein the mutation-specific primer is selected from any one of SEQ ID NOs: 8, 9, 10 or 11, the paired primer is selected from SEQ ID NO: 12, and the oligoblocker is selected from SEQ ID NO: 13 or 14.

29. The method of claim 27, wherein the mutation is EGFR T790M and wherein the mutation-specific primer is selected from SEQ ID NO: 1 or 2, the paired primer is selected from SEQ ID NO: 3 or 4, and the oligoblocker is selected from SEQ ID NO: 5.

30. The method of claim 27, wherein the mutation is EGFR L861Q and wherein the mutation-specific primer is selected from SEQ ID NO: 17 or 18, the paired primer is selected from SEQ ID NO: 19, and the oligoblocker is selected from SEQ ID NO: 20 or 21.

31. The method of claim 27, wherein the mutation is EGFR S768I and wherein the mutation-specific primer is selected from SEQ ID NOs: 34 or 35, the paired primer is selected from SEQ ID NO: 36, and the oligoblocker is selected from SEQ ID NO: 37.

32. The method of claim 27, wherein the mutation is EGFR G719S and wherein the mutation-specific primer is selected from SEQ ID NO: 24 or 25, the paired primer is SEQ ID NO: 26, and the oligoblocker is selected from SEQ ID NOs: 27 or 28.

33. The method of claim 27, wherein the mutation is EGFR G719A and wherein the mutation-specific primer is selected from any one of SEQ ID NOs: 31, 32 or 33, the paired primer is SEQ ID NO: 26 (G719 P19) and the oligoblocker is SEQ ID NO: 27 or 28.

34. The method of claim 27, wherein the mutation is EGFR exon19 Δ(E746-A750) and wherein the mutation-specific primer is SEQ ID NO: 40, the paired primer is SEQ ID NO: 41, and the oligoblocker is SEQ ID NO 42.

35. The method of claim 23, which comprises amplifying a second non-mutated target sequence on the EGFR gene, said second target sequence being located between 100 and 400 bp from the first target sequence on said gene, the first and second amplified sequences each being 50-95 nt in length and of a similar length, and wherein the method comprises determining a ratio of the amount of sequence amplified from the first and second sequence.

36. The method of claim 23, wherein:
the mutation is EGFR T790M, the first set of reagents comprises a mutation-specific primer selected from any one of SEQ ID NOs: 8, 9, 10 or 11, a paired primer selected from SEQ ID NO: 12, and an oligoblocker selected from SEQ ID NO: 13 or 14, and the second set of reagents comprises a first primer SEQ ID NO: 6 and a second primer SEQ ID NO: 7; and/or
the mutation is EGFR L858R, the first set of reagents comprises a mutation-specific primer selected from SEQ ID NO: 1 or 2, a paired primer selected from SEQ ID NO: 3 or 4, and an oligoblocker selected from SEQ ID NO: 5, and the second set of reagents comprises a first primer SEQ ID NO: 15 and a second primer SEQ ID NO: 16; and/or
the mutation is EGFR L861Q, the first set of reagents comprises a mutation-specific primer selected from SEQ ID NO: 17 or 18, a paired primer selected from SEQ ID NO: 19, and an oligoblocker selected from SEQ ID NO: 20 or 21, and the second set of reagents comprises a first primer SEQ ID NO: 22 and a second primer SEQ ID NO: 23; and/or
the mutation is EGFR S768I, the first set of reagents comprises a mutation-specific primer selected from SEQ ID NO: 34 or 35, a paired primer is selected from SEQ ID NO: 36, and an oligoblocker selected from SEQ ID NO: 37, and the second set of reagents comprises a first primer SEQ ID NO: 38 and a second primer SEQ ID NO: 39; and/or
the mutation is EGFR G719, the first set of reagents comprises a mutation-specific primer selected from SEQ ID NO: 24, 25, 31, 32 or 33, a paired primer selected from SEQ ID NO: 26, and an oligoblocker selected from SEQ ID NOs: 27 or 28; and/or
the mutation is EGFR DEL19, the first set of reagents comprises a mutation-specific primer selected from SEQ ID NO: 40, a paired primer selected from SEQ ID NO: 41, and an oligoblocker selected from SEQ ID NO: 42, and the second set of reagents comprises a first primer SEQ ID NO: 43 and a second primer SEQ ID NO: 44.

37. A kit for determining the presence of a mutation in the EGFR gene in a sample of a biological fluid containing cell free nucleic acids from a subject, the kit comprising at least a first set of reagents, wherein the first set of reagents amplifies a first target sequence from the EGFR gene, said first target sequence containing a site of a mutation in the EGFR gene, said first set comprising (i) a mutation-specific primer of 15-25 nt in length, (ii) a paired primer of 15-25 nt in length positioned such that the amplified first target sequence is 50-95 nt in length, and (iii) an oligoblocker, wherein the oligoblocker hybridizes specifically with the first target sequence in non-mutated form and blocks polymerase elongation.

38. The kit of claim 37, which comprises a second set of reagents (b) which amplifies a second non-mutated target sequence from the EGFR gene, said second target sequence being located between 100 and 400 bp from the first target sequence on said gene, and wherein said amplified second target sequence is 50-95 nt in length and of a length similar to that of the first amplified target sequence.

39. The kit of claim 38, wherein both sets are packaged in separate containers.

40. The kit of claim 37, which further comprises instructions for performing an amplification reaction and/or a polymerase, a buffer and/or nucleotides.

41. The kit of claim 37, which comprises several sets of reagents for determining the presence of several different mutations in the EGFR gene, the mutations T790M and L858R, or the mutations T790M and L858R and exon19 Δ(E746-A750).

42. A nucleic acid molecule consisting of any one of SEQ ID NOs: 1-55.
